(19) United States
(12) Patent Application Publication
(10) Pub. No.: US 2015/0080785 A1
(43) Pub. Date: Mar. 19, 2015

(54) ALZHEIMER'S DISEASE TREATMENT WITH MULTIPLE THERAPEUTIC AGENTS DELIVERED TO THE OLFACTORY REGION THROUGH A SPECIAL DELIVERY CATHETER AND IONTOPHORESIS

(71) Applicant: Wedge Therapeutics, LLC, St. Paul, MN (US)

(72) Inventor: Totada R. Shantha, Stone Mountain, GA (US)

(21) Appl. No.: 14/491,392

(22) Filed: Sep. 19, 2014

Related U.S. Application Data
(63) Continuation of application No. 13/945,087, filed on Jul. 18, 2013.
Application No. 13/945,087 is a continuation of application No. 13/473,454, filed on May 16, 2012.

(51) Int. Cl. A61N 1/05; A61N 1/30

(52) U.S. Cl. CPC A61N 1/0546; A61N 1/303
USPC 604/21; 607/135

(57) ABSTRACT

This invention describes the administration of multiple therapeutic agents with insulin in conjunction with bexarotene, ketamine, monoclonal antibodies Etanercept, IGF-1, and acetylcholine esterase inhibitors physostigmine, for treatment of Alzheimer's disease and other neurodegenerative diseases. Insulin, improves memory; also augments and amplifies the effects of the adjuvant therapeutic agents (paracrine and intracrine effects) and consequently reduces the β amyloid, its soluble precursors, prevents damage to the neuronal skeletal network (taupathy), and blocks glutamate excitotoxicity, reduces brain inflammation, prevents apoptosis, and increases the acetylcholine levels in the neurons and synapses; by using a combination of insulin, bexarotene, ketamine, Etanercept, IGF-1, and physostigmine therapeutic agents. The results are achieved by using the specially designed Iontophoresis incorporated olfactory mucosal delivery (ORE) catheter device located at the olfactory nerves, sphenoid sinus, and adjacent structures described here, to transport the large molecules of therapeutic agents to treat AD delivered to the CNS bypassing BBB from ORE.

### FIELD OF THE INVENTION

Alzheimer's disease (AD) is a chronic progressive neurodegenerative brain disease—syndrome of the aging. It is a major contributor to morbidity and modality in the elderly in nearly 5 million Americans. AD accounts for 70% of all cases of dementia. This invention described here relates to methods of treating Alzheimer's neurodegenerative diseases of the central nervous system (CNS) by the delivery of appropriate multiple therapeutic agents. Multiple therapeutic agents are delivered through the olfactory mucosa (ORE), olfactory nerves, sub Perineural epithelial, and nerve fascicular interstitial spaces, olfactory bulb, entorhinal cortex, trigeminal nerve, cranial nerves I, II, III, IV, and VI on the wall of the sphenoid sinus, sphenopalatine ganglion afferent and efferent nerves, cranial-vertebral venous system (CVVS), and circumventricular organs (CVO). These combined therapeutic agents of this invention are delivered to the brain and brainstem affected by Alzheimer's disease, bypassing the blood brain barrier (BBB) through a special delivery catheter which incorporates Iontophoresis and electroporation.

### BACKGROUND OF THE INVENTION

Alzheimer's disease (AD) is one of the common forms of neurodegenerative diseases resulting in dementia, also known as senile dementia of the Alzheimer type and primary degenerative dementia of the Alzheimer's type, Alzheimer disease (AD). The dementia is a huge public health concern, with a new case diagnosed somewhere in the world every 7 seconds. It described by German psychiatrist and neuropathologist Alois Alzheimer in 1906 and named after him. There is no cure for the disease, which worsens as it progresses, and eventually leads to death within 7 years. Less than three percent of individuals live more than fourteen years after diagnosis. People diagnosed as having AD are usually over 65 years of age diagnosed by standard verbal and visual memory tests, decision-making and problem-solving tasks. In 2006, there were 26.6 million sufferers worldwide and 5 million of them in the USA. Alzheimer's disease predicted to affect 1 in 85 people globally by 2050. Early symptoms often erroneously thought to be ‘age-related’ concerns, or manifestations of stress. When AD suspected, the diagnosis usually confirmed with tests that evaluate behavior, memory, cognition, and thinking abilities, followed by brain scan studies.

The neurodegenerative diseases divided into two all-encompassing wide categories of brain afflictions. The diseases are imprecisely divided into two groups—1. Conditions affecting memory that are ordinarily related to dementia such as Alzheimer's disease and 2. Conditions causing problems with movements such as Parkinson's. The most widely known neurodegenerative diseases include Alzheimer (or Alzheimer's) disease along with its precursor mild cognitive impairment (MCI), Parkinson's disease (including Parkinson's disease dementia), and multiple sclerosis and a host of others. Less well-known neurodegenerative diseases include dozens of names in a comprehensive listing found at the web site (www) of the National Institute of Neurological Disorders and Stroke (NINDS) of the National Institutes of Health (NIH) of the United States government (GOV) in a subdirectory (Idisorderidisordecindex) web page (htm). It is understood that such diseases often go by more than one name and that a nosology may oversimplify pathologies that occur in combination or that are not archetypical or standard. Certain other disorders, such as postoperative cognitive dysfunction; described only recently, and they too may involve neurodegeneration after anesthesia and surgery. Other disorders such as epilepsy may not be primarily neurodegenerative, but at a particular point in the progression of the disorder, it might involve nerve degeneration.

Despite the fact that at least some aspect of the pathology of each of the neurodegenerative diseases mentioned above is different, their pathologies and symptoms that they have in common often make it possible to treat them with similar therapeutic agents and methods. Hence, the invention described herein can be used with selected multiple therapeutic agents as described, to treat the majority of these neurodegenerative diseases. Many publications describe features that neurodegenerative diseases have in common (Dale E. Bredesen, Rammohan V. Rao and Patrick Mehlen. Cell death in the nervous system. Nature 443 (2006): 796-802; Christian Haass. Initiation and propagation of neurodegeneration. Nature Medicine 16 (2010): 1201-1204; Michael T. Lin and M. Flint Beal. Mitochondrial dysfunction and oxidative stress in neurodegenerative diseases. Nature 443 (2006) 787-795).

Our focus is on AD in particular. The AD disease symptoms can include confusion, irritability, aggression, mood swings, trouble with language, and long-term memory loss. The sufferer often withdraws from family and society. AD is a degenerative incurable disease that the sufferer relies on others for assistance and care. The caregiver is usually one of the family members, a spouse, or close relatives, placing a great burden on them, and is one of the most costly diseases to the society and family.

The cause and progression of Alzheimer's disease is not well understood. Research shows that the disease is associated with plaques and tangles in the brain. Current treatments only help with the symptoms of the disease. There are no available treatments to stop or reverse the progression of the disease. As of 2008, more than 500 clinical trials have been conducted to find ways to treat the disease, but it is unknown if any of the tested treatments will work. Mental stimulation, exercise, NSAID intake, and a balanced diet suggested as possible ways to delay symptoms in healthy older individuals. However, they are not proven as effective treatments once the symptoms develop.

The course of the disease divided into four stages, with progressive patterns of cognitive and functional impairments. 1. Pre-dementia; 2. Mild early Start of the disease; 3. Moderate progressive deterioration; 4. Severe or advanced—the last stage in which a person is completely dependent and bed ridden.

Alzheimer's disease is characterized by the accumulation of neurofibrillary tangles (tau-τ-protein) and neuritic plaques (amyloid β) in the brain affecting especially the degeneration of neurons in the olfactory bulb and its connected brain structures. They are entorihinal cortex (EC), the hippocampal formation, amygdaloid nuclei, nucleus basalis of Meynert, locus ceruleus, and the brainstem raphe nuclei all of which project to the olfactory bulb (FIG. 14). The degenerative changes result in the loss of memory and cognitive function. There is a major loss of cortical and hippocampal choline acetyltransferase activity and degeneration of the basal forebrain cholinergic neurons. Loss of smell in Alzheimer's is due to necrosis and/or apoptosis of olfactory neurons, olfactory bulbs, olfactory tracts, the pre-pyriform cortex and the entorihinal cortex.

Etiology and Neuro-pathophysiology: The cause for most Alzheimer's cases is unknown. The amyloid hypothesis postulated that amyloid beta (Aβ) deposits are the essential cause of the disease. Also APOE4, the major genetic risk factor for AD, leads to excess amyloid buildup in the brain before AD symptoms arise. Thus, Aβ deposition precedes clinical AD. Interestingly, an experimental vaccine found to clear the amyloid plaques in early human trials, but it did not have any significant effect on dementia. Studies showed that a close relative of the beta-amyloid protein, and not necessarily the beta-amyloid itself, may be a major culprit in the disease. A 2004 study found that deposition of amyloid plaques does not correlate with neuronal loss and memory loss. This observation supports the tau hypothesis; the theory and proposal that tau protein abnormalities initiate the disease cascade. Eventually, they form neurofibrillary tangles inside nerve cell bodies resulting in the microtubules' disintegration, collapsing the neuron's transport system, causing malfunctions in biochemical communication between neurons and later in the death of the cells. Herpes simplex virus type 1 is proposed to play a causative role in people carrying the susceptible versions of the ApoE gene. Another hypothesis asserts demyelination in the aged leads to axonal transport disruptions, leading to loss of neurons. Iron released during myelin breakdown and its vascular complex has been hypothesized, and implicated as a causative factor. I do believe that the disruption of BV with release of iron from the hemoglobin around the myelin and neuropil, resulting in the iron catalyzed hydrogen peroxide called Fenton's reaction leads to generation of reactive oxygen species (ROS) during these demyelization episodes that can have an adverse effect on the neurons resulting in their apoptosis resulting in Alzheimer's. There is a possibility that it may also play a role in development of MS. We did treat MS patients with Deferoxamine chelation, high dose vitamin B complex, and massive doses of IV Vitamin B₁, liver extract, and Vitamin B12 along with hyperbaric therapy. The symptoms disappeared, for one to three months, and the treatment repeated. One the patients we treated had massive lesions in the brain and the cervical spinal cord. After a month of treatment, the lesions disappeared, and she is still functional. She completed her PhD after recovery and gave birth to a healthy baby. Hence, we believe the chelation of the iron from the CNS should be one part of the therapy in the treatment of Alzheimer's and other degenerative diseases. Oxidative stress and dyshomeostasis of biometal metabolism may be significant in the formation of the pathology. We already have the therapeutic agent Deferoxamine that binds to the iron (chelate) locally or through circulation. We already are planning to use iron chelation by administering deferoxamine to olfactory mucosa or parenteraly with insulin to extract iron in the treatment of Alzheimer's and other neurodegenerative diseases.

Interestingly, the AD individuals display 70% loss of locus coeruleus cells that provide norepinephrine. Locus coeruleus cells are located in the pons, projects and innervate spinal cord, the brain stem, cerebellum, hypothalamus, the thalamic relay nuclei, the amygdala, the basal telencephalon, and the cortex. The norepinephrine from the LC has an excitatory effect on most of the brain, mediating arousal and priming the brain's neurons activated by stimuli. The norepinephrine from this nucleus stimulates microglia to suppress Aβ-induced production of cytokines and their phagocytosis of Aβ suggesting degeneration of the locus ceruleus might be responsible for increased Aβ deposition in AD brains initially. This nucleus in the pons (part of the brainstem) is involved with physiological responses to stress and panic, and is the principal site for brain synthesis of norepinephrine (noradrenalin) besides the adrenal glands.

Studies point out the accumulation of beta amyloid peptides as the central event triggering neuron degeneration. Accumulation of aggregated amyloid fibrils, are believed to be the toxic form of the protein responsible for disrupting the cell's calcium ion homeostasis, and induce programmed cell death (apoptosis). It is also known, that Aβ selectively builds up in the mitochondria in the cells of Alzheimer's-affected brains, and it inhibits certain enzyme functions and the utilization of glucose by neurons. It is in the glucose utilization pathology where the administration of olfactory mucosal insulin along with other therapeutic agents plays an important role in the treatment of Alzheimer's disease described in this invention.

Various inflammatory processes and cytokines may also have a role in the pathology of Alzheimer's disease; hence, we also use monoclonal antibodies to counter the adverse effects of cytokines; in which the cytokine antagonist provides the patient with the chance to heal, slows disease progression, or at the very least improves the patient's CNS health. Alterations in the distribution of different neurotrophic factors and in the expression of their receptors such as the brain derived neurotrophic factor (BDNF) have been described in AD. Our invention will take into consideration all these causative factors in treating the disease with the use of the IGF-1 neurotrophic factor and monoclonal antibodies to counter the inflammation induced cytokine in causation and progression of AD.

Neuropathology: Alzheimer's disease is characterized by loss of neurons and synapses in the cerebral cortex and certain subcortical regions, with gross atrophy of the temporal lobe, parietal lobe, parts of the frontal cortex and cingulate gyrus with loss of acetylcholine. Studies using MRI and PET scans have documented reductions in the size of specific brain regions in people with Alzheimer's. Both amyloid plaques and neurofibrillary tangles are clearly visible by microscopy in brains of those afflicted by AD. Plaques are insoluble deposits of amyloid-beta (Aβ) peptide and cellular material outside and around neurons.

Alzheimer's disease has also been recognized as a protein misfolding disease (proteopathy), caused by accumulation of abnormally folded Amyloid beta and tau proteins in the brain. Plaques are made up of small peptides, 39-43 amino acids in length, called beta-amyloid (also written as A-beta or Aβ). Beta-amyloid is a fragment from a larger protein called amyloid precursor protein (APP), a transmembrane protein that penetrates through the neuron's membrane. APP is a membrane protein that is concentrated in the synapses of neurons. APP is the precursor molecule whose proteolysis generates β amyloid, a peptide whose amyloid fibrillar form is the primary component of amyloid plaques found in the brains of AD patients. APP is critical to neuron growth, survival, and post-injury repair. In Alzheimer's disease, an unknown process causes APP to divide into smaller fragments by enzymes through proteolysis and these fragments give rise to fibrils of beta-amyloid, which form clumps that deposit outside neurons in dense formations known as senile plaques.

AD is also regard as a tauopathy also due to the abnormal aggregation of the tau protein within the neurons and its neurotubules. Tau proteins are abundant in the central nervous system, and they stabilize microtubules. When tau proteins are defective and no longer available for proper stabilization of microtubules, it results in the neuronal cytoskeleton falling apart, contributing to neuronal malfunction and cell death. Defective tau proteins will aggregate and twist into neurofibrillary tangles (NFTs), so that the protein is no longer available for the stabilization of microtubules.

All neurons have a cytoskeleton, an internal support structure partly made up of structures called microtubules. These microtubules act as railroad tracks, guiding nutrients and molecules from the body of the neuronal cell to the ends of the axon and back. A protein called tau stabilizes the microtubules. In AD, tau undergoes biochemical changes, becoming hyperphosphorylated; it then begins to pair with other protein threads, creating neurofibrillary tangles and disintegrating the neuron's transport system.

It is known that the Inflammation with the immune system plays a momentous role in AD pathogenesis. The inflammatory mechanisms in AD involve microglia, astrocytes (astroglia), the complement system, and various inflammatory mediators (including cytokines and chemokines). Microglial cells are the inhabitant immune cells in the brain. They are thought to contribute to neuronal decay and death in AD by secretion of neurotoxins (cytokines). It is important to note that when microglia are activated during inflammation, they also secrete a host of inflammatory mediators including cytokines (TNF, interleukins, IL-l˜and IL-6) and chemokines (macrophage inflammatory protein MIP-la, monocyte chemoattractant protein MCP-I and interferon inducible protein IP-10) that promote the inflammatory flame. Microglial cell activation and migration toward Aβ plaques precede the appearance of abnormally shaped neurites and the formation of neurofibrillary tangles. Elevated levels of TNF-alpha also induce an increased expression of interleukin-I, which in turn increases production of the precursors that may be necessary for formation of Aβ plaques and neurofibrillary tangles. Thus, the secretion of TNF-alpha by microglia contributes to a cycle wherein tau aggregates to form tangles, and a vicious cycle of AD pathology ensues. Further TNF-alpha is shown to mediate the disruption in synaptic memory mechanisms. All these various pathologic processes make the AD a complex disease difficult to pinpoint its etiology, and find a cure.

Exactly how production and aggregation of the beta amyloid peptide gives rise to the pathology of AD even now not known. Accumulation of aggregated amyloid fibrils, which are believed to be the toxic form of the protein responsible for disrupting the cell's calcium ion homeostasis, induces programmed cell death (apoptosis). It known that Aβ selectively builds up in the mitochondria in the cells of Alzheimer's-affected brains, and it inhibits certain enzyme functions and the utilization of glucose by neurons (Chen X, Yan S D. 2006. “Mitochondrial Aβ: a potential cause of metabolic dysfunction in Alzheimer's disease”. IUBMB Life 58 (12): 686-94). This hypothesis was supported by our work that the 2-4-dinitrophenol reduces these pathological changes and improves the memory and cognition in Lyme disease and senile type dementia, and early Alzheimer's disease (unpublished data 2004). We had to discontinue the study due to federal restriction on such use that is not FDA approved. The dose we used was minuscule and the benefits were many, without a single complication.

Alzheimer's disease is diagnosed clinically from the patient history, collateral history from relatives, clinical observations, advanced medical imaging with computed tomography (CT) or magnetic resonance imaging (MRI), and with single photon emission computed tomography (SPECT) or positron emission tomography (PET) scan.

The U.S. Food and Drug Administration (FDA) and the European Medicines Agency (EMA) have approved a number of therapeutic agents to treat the cognitive manifestations of Alzheimer's symptomatically. Three of them are acetyl cholinesterase inhibitors and the other is memantine, an NMDA receptor antagonist. There is no drug for delaying or halting the progression of the disease. Reduction in the activity of the cholinergic neurons is well-known and the Acetyl cholinesterase inhibitors are employed to reduce the rate at which acetylcholine (ACh) is broken down, thereby increasing the concentration of ACh in the brain and combating the loss of ACh caused by the death of cholinergic neurons to enhance memory and cognition. The cholinesterase inhibitors approved for the management of AD symptoms are: donepezil (brand name Aricept™), galantamine (Razadyne™), and rivastigmine (branded as Exelon and Exelon™ Patch). The use of these drugs in mild cognitive impairment has not shown any effect in a delay of the onset of AD.

Glutamate is a excitatory neurotransmitter of the nervous system, and excessive amounts in the brain can lead to cell death through a process called excitotoxicity which consists of the over stimulation of glutamate receptors. Excitotoxicity occurs not only in Alzheimer's disease, but also in other neurological diseases such as Parkinson's disease and multiple sclerosis. Memantine (brand names Akatinol, Axura, Ebixa/Abixa, Memox and Namenda™), is a noncompetitive NMDA receptor antagonist first used as an anti-influenza agent. It acts on the glutamatergic system by blocking NMDA receptors and inhibiting their overstimulation by glutamate. In our invention, we administer olfactory mucosal ketamine as a NMDA blocker, which is easy to use and effective. Antipsychotic drugs are modestly useful in reducing aggression and psychosis. Mini doses of ketamine can serve a similar function in reducing depression and blocking NMDA receptors. Even today, there is no cure for Alzheimer's disease and the cause and progression of Alzheimer's disease proceed unabated due to the accumulation of plaques and tangles in the brain (Tiraboschi P, Hansen L A, Thal L J, Corey-Bloom J. The importance of neuritic plaques and tangles to the development and evolution of AD. Neurology. 2004; 62 (11):1984-911).

Anti-inflammatory agents could prove useful in AD treatment by toxicity reduction. Nonsteroidal anti-inflammatory drugs (NSAID) such as ibuprofen, indomethacin, and sulindac sulfide decrease the amount of Aβ1-42. Neuronal death associated protein kinase (DAPK) inhibitors such as derivatives of 3-amino pyridazine could modulate the neuroinflammatory responses in astrocytes by Aβ activation.

Most mutations in the APP and presenilin genes increase the production of a small protein called Aβ42, which is the main component of senile plaques. The top known genetic risk factor is the inheritance of the ε4 allele of the apolipoprotein E (APOE). Between 40 to 80% of people with AD possess at least one APOEε4 allele that increases the risk of the disease by three times. Over 400 genes have been tested for association with AD, most with unacceptable or uncertain results.

Cyclooxygenases (COX-l and -2) inhibitors, antioxidants such as vitamins C and E, as well as modulators of NMDA such as memantine could also reduce the cellular toxicity of Aβ. The MAO inhibitors Rasagiline, selegiline (Anipryl, L-deprenyl, Eldepryl, Emsam, Zelapar®), and tranylcypromine are also known to delay the further deterioration of cognitive functions in more advanced forms in Alzheimer's, nevertheless the pathology progresses unabated. These simple therapeutic agents incorporated in the treatment of Alzheimer's disease described here. All our AD patients using our method of therapy described here, were put on oral intake of Cyclooxygenases (COX-l and -2) inhibitors, antioxidants, such as vitamins C, D, and E, magnesium L threonate, Zinc, and statins. Experiments show that ingesting one gram of omega-3, per day equal to approximately half a fillet of salmon per week, is associated with 20 to 30 percent lower blood beta-amyloid levels. For this reason, add this supplement, eat Salmon weekly to maintain brain health, and prevent or delay the onset of Alzheimer's disease.

Etanercept, a biologic antagonist of TNF-alpha, a potent anti-TNF fusion protein delivered by perispinal Etanercept administration, has shown to improve the cognitive abilities of AD patients (Edward L Tobinick and Hyman Gross. Rapid cognitive improvement in Alzheimer disease following perispinal Etanercept administration. Journal of Neuroinflammation 2008, 5:2. W Sue T Griffin. Perispinal etanercept: Potential as an Alzheimer therapeutic. Journal of Neuroinflammation. 2008, 5:3; Edward Tobinick. Tumour Necrosis Factor Modulation for Treatment of Alzheimer's Disease Rationale and Current Evidence. CNS Drugs 2009; 23 (9): 713-725. Richard C. Chou, Michael A. Kane, Shiva Gautam and Sanjay Ghirmire. Tumor Necrosis Factor Inhibition Reduces the Incidence of Alzheimer's disease in Rheumatoid Arthritis Patients. Abstracts of the American College of Rheumatology, Nov. 8, 2010, Atlanta Ga., Presentation No. 640). Our study showed that the cervical epidural and intranasal ORE delivery of Etanercept is much more effective in the treatment of AD compared to perispinal, or epispinal or interspinal routes of administration. It is transported to the CNS through the CSF, not by direct spread by cervical vertebral venous system as perceived (Shantha T R and Evans J A: Arachnoid Villi in the Spinal Cord, and Their Relationship to Epidural Anesthesia. Anesthesiology 37:543-557, 1972).

Magnesium-L-threonate (MgT): Magnesium is known as a key nutrient for optimal brain function. Scientists have found it promotes learning and memory because of its beneficial effect on synaptic plasticity and density. Magnesium works with calcium to modulate “ion channels” that open in response to nerve impulses, which in turn trigger neurotransmitter release. The most important aspect of these channels is controlled by a complex called the NMDA receptor. NMDA receptors play an important role in promoting neural plasticity and synaptic density, the structural foundations of memory. Magnesium deficiency can cause symptoms ranging from apathy and psychosis to memory impairment. Insufficient magnesium slows brain recovery following injury from trauma and in laboratory studies accelerates cellular aging. Experimental studies show that the magnesium elevation in brain tissue observed in MgT supplementation increases the number of functioning neurotransmitter release sites, and it enhances synaptic density and plasticity, the structural basis of learning and memory. In numerous experimental models, supplementation with magnesium-L threonate has been shown to enhance memory and cognitive performance in multiple tests (Martin Alessio. Novel magnesium compound halts neurologic decay. February 2012. Life Extension, pages 31-34). All our patients with cognitive and memory problems who received MgT supplementation showed improvement in memory and cognition.

The use of estrogen by postmenopausal women has been associated with a decreased risk of AD. Women using hormone replacement had about a 50% reduction in disease risk. Estrogen is found to exert antiamyloid effects by regulating the processing of the amyloid precursor protein (APP) in the gamma secretase pathway. In our clinic, all the postmenopausal women prescribed with hormone replacement therapy for this reason, if there was no other contraindication. We have used this hormone (progesterone) with insulin combined with or without IGF-1 and Monoclonal antibodies as olfactory mucosal spray for the treatment of PTSD, strokes, for patients with memory loss and cognition especially in menopausal woman.

Lipid-lowering agents (3-hydroxy-3-methyglutaryl coenzyme A (HMG-CoA) reductase inhibitors) or statins are associated with lower risk of AD. Hence, we prescribed these statins in patients above 65 years of age. Statins were shown to reduce the intra and extracellular amount of Aβ peptide. These agents include lovastatin (Mevicor®), pravastatin (Pravachol®), atorvastatin (Lipitor simvastatin (Zocor®), fluvastatin, cerivastatin, rosuvastatin (Crestor®), compactin, mevilonin, mevastatin, visastatin, velostatin, synvinolin, rivastatin, itavastatin, and pitavastatin. All our patients above the age of 60 and with early decline in memory; and those with higher levels of blood cholesterol received statin drugs as part of the therapy whether diagnosed with AD or not.

Interferons are cytokines, i.e. soluble proteins that transmit messages between cells and play an indispensable role in the immune system by helping to destroy microorganisms that cause infection and repairing any resulting damage. They are naturally secreted by infected cells and were first identified in 1957. Their name derived from the fact that they “interfere” with viral replication and production. Interferons exhibit both antiviral and anti-proliferative activity. Based on biochemical and immunological properties, the naturally-occurring human interferons are grouped into three major classes: interferon-alpha (leukocyte), interferon-beta (fibroblast) and interferon-gamma (immune). The three major IFNs referred to as IFN-α, IFN-β and IFN-γ. Alpha-interferon is currently approved in the United States and other countries for the treatment of hairy cell leukemia, venereal warts, Kaposi's Sarcoma (a cancer in patients with Acquired Immune Deficiency Syndrome (AIDS)), and chronic non-A, non-B hepatitis. It has been shown that IFN-β is a potent promoter of nerve growth factor production by astrocytes, and based on this observation it was suggested that IFN-β might have a potential utility in AD, but no experimental data is available. U.S. Patent Application Publication Number: 2007/0110715 A1 describes the use of interferon-β (IFN-β); for treating and/or preventing Alzheimer's disease (AD), Creutzfeld-Jakob disease (CJD) or Gerstmann-Straussler-Scheinker disease (GSSD). The interferon-β added to the olfactory nerve delivery along with other therapeutic agents such as insulin, bexarotene, and ketamine, monoclonal antibodies, IGF-1, and cholinesterase inhibitor therapeutic agents described in this invention. It is used as a spray with insulin every other day at a dose of up to 10 μg per spray per day delivered directly to the olfactory nerve mucosal area (ORE), not to the respiratory mucosa (see FIG. 1, 1a).

Lilly® Drug Company is conducting phase III clinical trials on a gamma secretase inhibitor. It is shown to decrease the amount of amyloid in sampled cerebral spinal fluid. Clinical trials hafted at present due to exacerbation of cognitive problems and an increase in the incidence of kin cancer in those taking it.

With the ability to diagnose AD in the early stages through use of modern diagnostic methods such as biomarkers, treatment of AD as described in this invention justifies the treatment at stages prior to definite dementia. Such an approach may still slow, stop, cure, curtail, or reverse the pathophysiological processes underlying AD and its progression.

Biomarkers to diagnose the AD are cognitive, physiological, biochemical and anatomical inconsistencies in scan studies that indicate the progression of AD. The most commonly measured biomarkers are decreased Aβ 42 in the cerebrospinal fluid (CSF), increased CSF tau, and decreased fluorodeoxyglucose uptake on PET (FOG-PET), PET amyloid imaging, and structural MRI measures of cerebral atrophy. Biomarkers of neuronal injury, dysfunction, and neurodegeneration become abnormal later in the disease. Degrees of cognitive symptoms not directly related to biomarkers of Aβ deposition, but the biomarkers of Aβ deposition become abnormal early in the disease.

The Blood Brain Barrier (BBB) and its Implications in the Treatment of CNS Dieseases Such as Alzheimer's

The problem in the treatment of CNS diseases including Alzheimer's is that 98% of the therapeutic agents are not transported, delivered, or passed on to the site of pathology in the brain. The BBB is responsible for creating a barrier for delivery of therapeutic agents to the brain and spinal cord. This formidable barrier is overcome by use of therapeutic agents using olfactory mucosa as the route of delivery. Talegaonkar and Mishra has an excellent review article on the subject of olfactory nerve (ORE) delivery of therapeutic agents to the CNS bypassing BBB which are incorporated herein (Talegaonkar, S, P. R. Mishra. Intranasal delivery: An approach to bypass the blood brain barrier. Indian J Pharmacol. June 2004, Vol 36, Issue 3, 140-147). The BBB is located in 400 miles of capillaries within the brain due to its unique histological make up compared to the other capillaries in other regions of the body. The endothelial cells of the blood vessels (BV) of the CNS differ from the peripheral capillary endothelial cells in the following histological differences such as:

I. Lack of fenestration in the endothelial cells: The endothelial cells joined by tight junctions, which block the protein molecule movement from within. In addition, they block the hydrophilic transfer of substances from the capillary to the CNS.
II. These tight endothelium junctions in the BBB are 100 times tighter than similar junctions of other systematic capillary endothelium (Butte A M, Jones H C, Abbot N J. Electrical resistance across the blood-brain barrier in anaesthetized rats; a development study. J Physiol 990; 429:47-62), and thus create a formidable barrier, which blocks almost 98% of the therapeutic agents delivered to the systemic circulation reaching the neuropile and neurons of CNS. That is why the olfactory nerve mucosal delivery (ORE) of therapeutic agents is the most important method of bypassing these tight junctions of the BBB, and delivering the agents directly to the CNS for the treatment of Alzheimer's disease and other neurodegenerative diseases.
III. The endothelial Cells contain specific a receptor transport system for a given molecule, such as insulin, glucose, glucagon etc. but not for most of the therapeutic agents used.
IV. They display net negative charge inside the endothelial cell and basement membrane impeding anionic molecules to cross the membrane,
V. they show paucity of pericytes in the wall of these BV,
VI. hardly any pinocytotic vesicles in the cytoplasm of the endothelial cells compared to peripheral endothelial blood vessels cells,
VII. Astrocytes foot process covers 95% of the endothelium outer surface,
VIII. There is a thick basement membrane encasing these brain capillaries completely,
IX. The cerebral vascular endothelial cell possesses a transcellular lipophilic pathway, allowing diffusion of small lipophilic compounds such as insulin, transferrin, glucose, purines, and amino acids.
X. The BBB prevents passage of ionized water-soluble compounds with a molecular weight greater than 180 Daltons. Many new neuro therapeutic agents have been discovered, but because of a lack of suitable strategies for drug delivery across the BBB, these agents are fruitless and only effective if methods to break the BBB are discovered.
XI. The concentration gradients also play a role in transport of therapeutic agents across systemic BV, but display hardly any such effect across the BBB BV of the CNS.

Due to the above-explained histological features of the brain blood vessels, they form a formidable BBB capillary system that is 400 miles long with iron clad tight junctions between endothelial cells within the human brain BV. Because of the above-explained histological embodiments, the brain capillaries prevent transport of most of the therapeutic agents (98%) from inside the BV. They also prevent and/or inhibit clearance of neurotoxic compounds such as beta amyloid and their precursor in Alzheimer's; reactive oxygen species (ROS), toxic metabolites and their derivatives from the CNS entering the systemic circulation for clearance and to provide the homeostatic neuropil milieu functional state for neuronal complexes. Hence, the brain keeps on accumulating toxins with no path to enter or passage to exit from the brain, contributing to the CNS afflictions. That is why the delivery of multiple anti Alzheimer's disease therapeutic agents directly through the olfactory mucosal region and other routes bypassing the BBB as described in this invention will be one of the most effective methods of treating Alzheimer's and other neurodegenerative diseases. Treatment with a single agent has proved to be the least effective method of treating Alzheimer's disease.

CNS and Peripheral nervous system has Virchow-Robin space, the extension of pia mater into SAS, into the outer surface of the brain and spinal cord and Perineural epithelium of peripheral nerves, as the BV enters the surface of the brain (and nerve fasciculi) for a short distance allowing the CSF to permeate with therapeutic agents (FIG. 13). This is not part of the BBB (Shantha T R: Peri-vascular (Virchow-Robin) space in the peripheral nerves and its role in spread of local anesthetics, ASRA Congress at Tampa, Regional Anesthesia 17 (March-April, 1992). It plays a role in the distribution of therapeutic agents to neuropil on the surface of the cerebral cortex from the CSF of the SAS by bypassing the BBB using olfactory nerves, other cranial nerves situated close to the olfactory mucosa, cranial valveless vertebral venous system plexus (CVVS), and associated structures described in this invention.

For the present other than physical and mental exercise, only symptomatic therapies for AD are available. The current described invention of multiple therapeutic strategies in AD treatment incorporated herein is a more effective system compared to the presently available symptomatic single agent therapeutic modalities to treat the AD. The aims of the present invention are as follows:

a) at lowering Aβ levels and decreasing levels of toxic Aβ aggregates through inhibition of the processing of amyloid precursor protein (APP) to Aβ peptide;

b) at inhibition, reversal or clearance of Aβ aggregation which are present in AD, and prevent their formation;

c) at cholesterol reduction, increase acetylcholine (Ach), reduce NMDA excitotoxicity, counter the inflammatory cytokine production by monoclonal antibodies, prevent neuronal apoptosis by neurotrophic factors;

d) at Aβ immunization to prevent its production, accumulation, and removal in the neuropil is the goal of the therapy for AD;

e) at reduction of ROS production and enhancing antioxidant activity with various nutriceuticals;

f) at Inhibition of inflammation in the brain, a root cause of the disease; g) at reducing the excitotoxicity of neurons which leads to neuronal apoptosis;

h) at Increasing the acetylcholine neurotransmitter in the brain; and

i) at increasing the heath of the neurons by administration of neurotrophic factors.

The present invention involves the use of above combination of therapeutic modalities to achieve these goals; in addition to existing physical and mental exercises, and symptomatic treatment of AD.

### SUMMARY OF THE INVENTION

The principal of the present invention for the treatment of Alzheimer's disease using multiple therapeutic agents encompasses:

a) Prevention of the breakdown of the amyloid precursor protein (APP) which forms Aβ of AD,
b) Preventing the amyloid β (Aβ) formation, and enhancing their removal,
c) Prevention of neurofibrillary tangles by the abnormal tau protein inside the nerve cells and their extensions in the neuro-skeletal network and nerve tubules,
d) Prevention of apoptosis of cholinergic and other neurons,
e) Prevention of loss of acetylcholine neurotransmitter and increase it in the neurons and at the synapses,
f) Prevention of inflammatory process in the brain (neuropil) responsible for initiation, and progression of the disease, and neuronal death.
g) Prevention of neuronal degeneration and apoptosis of neurons by providing neurotrophic factors,
h) Use of a special catheter delivery system, whereby the therapeutic agents are deposited on the olfactory nerve mucosal region (ORE), and their uptake enhanced by Iontophoresis to directly deliver therapeutic agents to the Alzheimer's disease afflicted CNS bypassing BBB.

It is the primary goal of this invention to treat Alzheimer's disease by administering multiple therapeutic agents through the intranasal olfactory mucosa, olfactory nerves, other cranial nerves (CN1-6), and cranial vertebral venous plexus route delivery of these therapeutic agents directly to the brain by passing the BBB.

It is the purpose of the present invention to provide a special catheter for delivery of therapeutic agents to the olfactory mucosal-nerve area (ORE), avoiding the spread to the respiratory mucosa for the maximum delivery of therapeutic agents into Alzheimer's disease afflicted brain through the olfactory nerves by passing BBB.

It is the goal of this invention to provide methods and the apparatus for delivery of measured therapeutic agents to the CNS neuropil, which are involved and affected in Alzheimer's diseases.

It is goal of this invention to deliver therapeutic agents through olfactory nerve, sub Perineural epithelial, and nerve fascicular interstitial spaces (SPES), the olfactory bulb, subarachnoid space (SAS), cranial vertebral venous plexus, and circumventricular organs that transport therapeutic agents to the CNS through CSF and nerve tracts entering and leaving the CNS.

It is the goal of this invention to deliver therapeutic agents through the olfactory nerves, olfactory bulb, and olfactory tract to the prefrontal cortex, the medial olfactory area, the temporal lobe, the lateral olfactory area, the entorhinal cortex, the hippocampus, the hypothalamus, brain stem nuclei, and cerebellum bypassing BBB.

It is a purpose of the present invention to provide a special catheter equipped with an Iontophoresis producing embodiment method to deliver large therapeutic agents molecules across the olfactory mucosa and olfactory nerve to the Alzheimer's disease afflicted central nervous system.

It is intent of this invention to deliver insulin to the Alzheimer's disease affected brain regions bypassing the blood brain barrier (BBB) through olfactory, trigeminal and sphenopalatine ganglion nerves routes, ten cranial nerves around the sphenoid sinus walls in the cavernous sinus, and cranial vertebral venous plexus (CVVS).

It is intent of this invention to deliver insulin with IGF-1 neurotrophic factor to the Alzheimer's disease affected brain regions bypassing the BBB through olfactory, trigeminal and sphenopalatine ganglion nerves routes, 10 cranial nerves around the sphenoid sinus walls, and cranial vertebral venous plexus (CVVS).

It is the intent of this invention to deliver an NMDA blocker ketamine and other such agents with insulin to the Alzheimer's disease affected brain regions bypassing the BBB through olfactory, trigeminal and sphenopalatine ganglion nerves routes, ten cranial nerves around the both sides of sphenoid sinus walls, and cranial vertebral venous plexus (CVVS).

It is the object of this invention to deliver bexarotene (TARGRETIN™) with insulin; to the Alzheimer's disease affected brain regions bypassing the BBB. These multiple therapeutic agents are delivered through olfactory, trigeminal and sphenopalatine ganglion nerves routes, 10 cranial nerves around the sphenoid sinus walls, circumventricular organs, and cranial vertebral venous plexus (CVVS) to prevent and reduce the amyloid beta (Aβ) plaques of the Alzheimer's disease afflicted neuropil, neurons, and their synaptic connections.

It is the intent of this invention, to deliver acetyl choline esterase blocker physostigmine, and related therapeutic agents with insulin, to the Alzheimer's disease (and chronic neurological disorders) affected brain regions bypassing the BBB. They are delivered through olfactory nerves, trigeminal nerve, sphenopalatine ganglion nerves, 10 cranial nerves around the sphenoid sinus walls, Cranial-Vertebral Venous System, and circumventricular organs routes to prevent the destruction of acetylcholine and increase their level in the neurons and their synapses to facilitate the nerve conduction that is lacking in the cholinergic neuron of the Alzheimer's disease afflicted brain.

This invention, by using olfactory nerves, trigeminal and other first five cranial nerves, sphenopalatine ganglion and it afferents and efferent's, Cranial-Vertebral Venous System, to SAS, CSF, Virchow-Robin space, and circumventricular organs with appropriate therapeutic agents; is intended to treat many neurodegenerative diseases besides Alzheimer's disease. The modality described here includes Alzheimer's disease, and other neurodegenerative diseases such as: Arachnoiditis, Autism, Brain Ischemia, CNS Infections, Cerebral Palsy, senile dementias, ALS, Cerebrovascular Disorders, Corticobasal Ganglionic Degeneration (CBGD) (not on MeSH), Creutzfeldt-Jakob Syndrome, Dandy-Walker Syndrome, Dementia, Encephalitis, Encephalomyelitis, Epilepsy, Essential Tremor, Friedreich Ataxia, Huntington Disease, Hypoxia Brain damage, Lewy Body Disease, Multiple sclerosis, Myelitis, Olivopontocerebellar Atrophies, PTSD, traumatic injury to the brain-blunt or otherwise, mental illnesses, Pantothenate Kinase Associated Neurodegeneration, Parkinson Disease, Parkinsonian Disorders, Postpoliomyelitis Syndrome, Prion Diseases, Pseudotumor Cerebri, Shy-Drager Syndrome, Spinal Cord Diseases, Stroke, Thalamic Diseases, Tic Disorders, Truett Syndrome, Uveomeningoencephalitic Syndrome, psychological disorders, addictions, and also in the treatment of cerebrovascular disorders such as stroke, PTSD, for the treatment of migraine, cluster and other types of headaches; post menopausal syndrome, postpartum depression, pain and other such diseases. Most importantly, this invention used in the treatment of neurodegenerative Alzheimer's disease, and other neurodegenerative disorders such as Parkinson's disease, Idiopathic Dementia, and ALS. These chronic neurological disorders treated using this invention include but are not limited to Alzheimer's Disease, Pick's disease, Creutzfeldt Jacob Disease (CJD), Variant CJD, Parkinson's Disease, Lewy Body Disease, Idiopathic Dementia, Amyotrophic Lateral Sclerosis (ALS), and the Muscular Dystrophies. These diseases curtailed with the use of the combination of therapeutic agents described in this invention.

The therapeutic, pharmaceutical, biochemical, and biological agents or compounds administered along with the above-described therapeutic agents and routes of delivery of this invention for the treatment of neurodegenerative and other diseases specific to the disease are many and diverse in nature. They are as follows: The chemotherapeutics, insulin, IGF-1, levodopa (5-10% crosses BBB) combined with a dopa decarboxylase inhibitor or COMT inhibitor, dopamine agonists and MAO-B inhibitors (selegiline and rasagiline)), Dopamine agonists (include bromocriptine, pergolide, pramipexole, ropinirole, piribedil, cabergoline, apomorphine and lisuride), non-steroidal anti-inflammatory drugs, acetyl cholinesterase inhibitors (such as tacrine, donepezil and the longer-acting rivastigmine; antibiotics), 2,4-dinitrophenol, glutamate receptor antagonist, glutathione, NMDA-receptor blocker such as ketamine, p amyloid inhibitor besides bexarotene, Alzheimer's vaccine, non-steroidal anti-inflammatory drug including COX-2 inhibitor, deferoxamine, hormones such as progesterone, enzymes, erythropoietin, Intranasal fibroblast growth factor, epidermal growth factor, microglial activation modulator, cholinesterase inhibitor, stimulant of nerve regeneration, nerve growth factor, non-steroidal anti-inflammatory drugs, interferon-β (IFN-β), antioxidants, Zinc and magnesium L. threonate with hormone, vitamin B₁₂, A, E, D₃, and B complexes, inhibitor of protein tyrosine phosphatase and similar therapeutic agents.

### BRIEF DESCRIPTION OF THE DRAWINGS

The present invention will be completely understood from the following detailed description of preferred embodiments thereof taken together with the drawings, in which:

FIG. 1 is the diagrammatic presentation 100 of the olfactory mucosa covering the medial and lateral walls of the nose, sphenopalatine ganglion, and anterior ethmoidal nerve.

FIG. 1a is the diagrammatic presentation 100a showing vestibule, respiratory and olfactory mucosa of the lateral and medial walls of the nose.

FIG. 2 is the diagrammatic presentation of the lateral wall 200 of the nerve structures in the nose.

FIG. 3 is the diagrammatic presentation of the medial wall 300 of the nerve structures in the nose.

FIG. 4 views of diagram 400 showing structure stimulated by electrical impulses transport to the CNS used in this invention.

FIG. 5 views of diagram 500 showing the inventive device used to stimulate olfactory mucosa.

FIG. 6 is the drawing 600 showing this inventive device in the olfactory mucosa with the tip in the sphenoid sinus.

FIG. 7 views of diagram 700 showing this inventive device in the olfactory mucosa with the tip in the sphenoid sinus with anchoring balloon.

FIG. 8 is the diagrammatic presentation 800 of the electrical stimulator directly to create Iontophoresis using this inventive device incorporating olfactory mucosal, sphenoid sinus, pituitary gland, sphenopalatine ganglion stimulators in one device.

FIG. 9 is the diagrammatic presentation 900 of the completely assembled electrical impulses delivering a catheter with balloon and inflating syringes.

FIG. 10 is the diagrammatic presentation 1000 showing the longitudinal section of the olfactory bulb, which conducts therapeutic agents to the cortical centers delivered through the olfactory nerves from the olfactory mucosa.

FIG. 11 is the diagrammatic presentation 1100 showing how the therapeutic agents transported to the olfactory bulb to CNS from olfactory mucosa.

FIG. 12 is the drawing of the section of the olfactory mucosa and electron micrograph of olfactory nerve fasciculi 1200 showing the sub Perineural epithelial space through which the therapeutic agents spread to CNS.

FIG. 13 is the diagram 1300 of the Virchow-Robin space in the central nervous system communicating with the SAS.

FIG. 14 is the diagrammatic presentation 1400 of the section of the olfactory mucosa and olfactory bulb and the transport of therapeutic agents to CNS.

FIG. 15 is the drawing of the location of the circumventricular organs 1500 that play a role in the passage of the therapeutic agents to the CNS due to CSF and vascular spread through the SAS and BV.

FIG. 16 is the drawing of the nerve fasciculi 1600 showing the Virchow-Robin space and subperineural epithelial space and entry of therapeutic agents to CNS.

FIG. 17 is the cross section of nerve fasciculi 1700 showing sub Perineural epithelial, and nerve fascicular interstitial spaces, and the entry of therapeutic agents to nerve fasciculi to be transported to the CNS.

FIG. 18 is the Histological diagram 1800 drawn after the light and electron microscopic study of the myelinated nerve axons with node of Ranvier within the nerve fasciculi, possible site of entry of therapeutic agents transported inside the axons.

FIG. 19 is the diagram 1900 of the neruopil structures between the ependymal linging of the central canal, ventricle, and the SAS surrounding the brain (CNS) and the spinal cord.

FIG. 20 is the diagrammatic presentation 2000 and 2000a of the special delivery inventive device used to dispense therapeutic agents at the olfactory mucosa and olfactory nerve (ORE) instead of the respiratory mucosa.

FIG. 21 is the diagrammatic presentation 2100 of the delivery catheter used to deliver therapeutic agents of the invention described herein, on the olfactory mucosa and olfactory nerve (ORE).

FIG. 22 is the diagram 2200 of the veins of the base of the brain and cervical vertebral anastomic veins showing the communication that forms the cranial vertebral venous plexus involved in the transport of therapeutic agents from ORE for the treatment of Alzheimer's disease and other neurodegenerative diseases.

FIG. 23 is the diagram 2300 of the veins of the base of the brain and vertebral anastomic veins showing the communication that forms the cranial vertebral venous plexus (CVVS) involved in transport of therapeutic agents delivered to epi and perispinal space (VVS).

FIG. 24 is Longitudinal section 2400 through spinal nerve roots from the monkey, showing an arachnoid villi 70 protruding into epidural veins 71 outside the dura into epidural veins.

FIG. 25 is a section through the human spinal root 2500, showing the arachnoid proliferation in the form of a villus 70 penetrating the dura 72 in close proximity to the epidural and perispinal veins 71.

FIG. 26 is the drawing 2600 of the histology of the spinal cord, dorsal and ventral roots, dorsal-root ganglion, and common nerve trunk and their membranes, and arachnoid villi in the nerve roots and their association with the epidural and perispinal valveless venous system as they emerge from the spinal and cranial nerve foramina.

### DETAILED DESCRIPTION OF THE INVENTION

### Description of the Terms Used in this Invention

As used in the specification and claims, the singular forms “a,” “an” and “the” include plural references unless the circumstance dictates otherwise. For example, the term “a cell” includes a plurality of cells.

The term “Alzheimer's” means Alzheimer's disease, Alzheimer's afflicted brain. The term is used to allude to “neurodegenerative diseases” “neurological diseases” “CNS diseases” such as Creutzfeld-Jakob disease, Parkinson's, senile brain atrophy, Gerstmann-Straussler-Scheinker disease, stroke, PTSD, Tumors, vascular disorders, and host of other such CNS afflictions.

The terms “apparatus” “device” “inventive device” are used interchangeably.

The terms “therapeutic,” “therapeutically effective doses,” and their cognates refer to those doses of a substance, e.g., of a protein, e.g., insulin, bexarotene, ketamine, monoclonal antibodies, AChEIs of an IGF-I, that result in prevention or delay of onset, or amelioration, of one or more symptoms of a disease such as Alzheimer's and Parkinson's.

The terms “therapeutic agents” and “therapy” imply to all drugs used to treat Alzheimer's and other associated diseases.

As used herein, the term “treating” or “treatment” and “example” refers to both therapeutic treatment, prophylactic or preventative measures and methods thereof.

“Neurotrophic” factors are agents that affect the survival and differentiation of neurons in the peripheral and central nervous systems.

A “subject,” “individual” or “patient” used interchangeably herein, refers to a vertebrate, preferably a mammal, more preferably a human. The term “mammal (s)” include but are not limited to, humans, mice, rats, monkeys, farm animals, sport animals, and pets.

As used herein the term “ameliorate” is synonymous with “alleviate,” “relief,” or “relieve” and means to reduce or ease signs and symptoms, cure, or curtail the disease processes.

The term “neuropil” “Neuropile” in the following description refers to an intricate, complex network of axons, dendrites, and glial branches that form the bulk of the central nervous system's grey matter with Microglial cells with BV endowed with BBB and in which nerve cell bodies with their synapses embedded.

The term “BBB” (blood brain barrier) refers to the 400 miles of blood vessels in the form of capillaries that supply the neuropil and form the bulk of the blood supply (20% of the cardiac output) of the central nervous system's gray matter in which the nerve cell bodies lay surrounded and embedded in the neuropile. The olfactory nerves, CVVS, and circumventricular organs provide a route bypassing the BBB, presenting the select therapeutic agents directly to the neuropile of the brain to the site of pathology to treat CNS diseases including Alzheimer's disease.

The term “Circle of Willis,” “CW” “Cerebral By,” or brain “BV” includes anterior cerebral arteries, anterior communicating arteries, internal carotid arteries, posterior cerebral arteries, the basilar artery and middle cerebral arteries supplying the brain and giving branches to and from the BBB capillaries inside the brain, brain stem, and spinal cord.

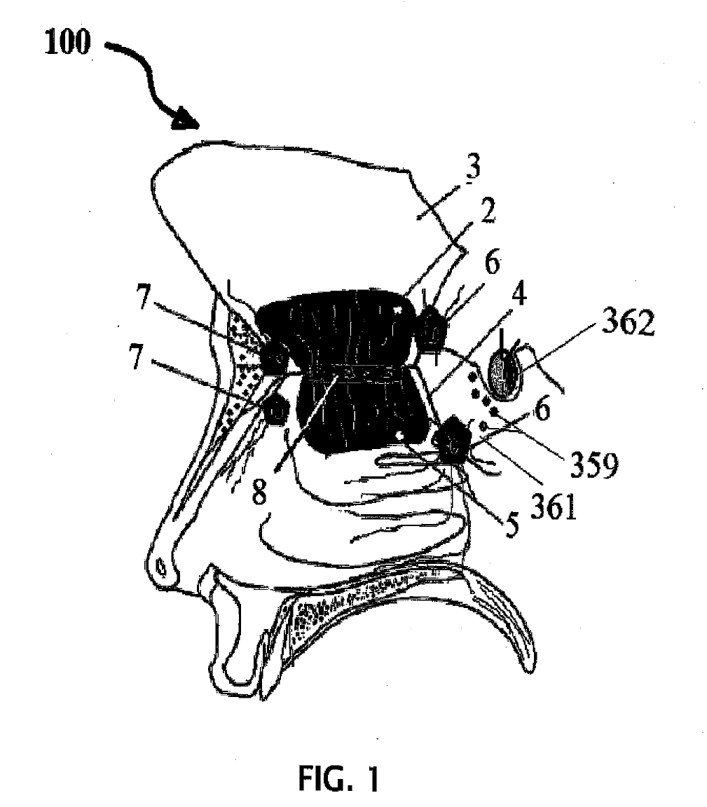

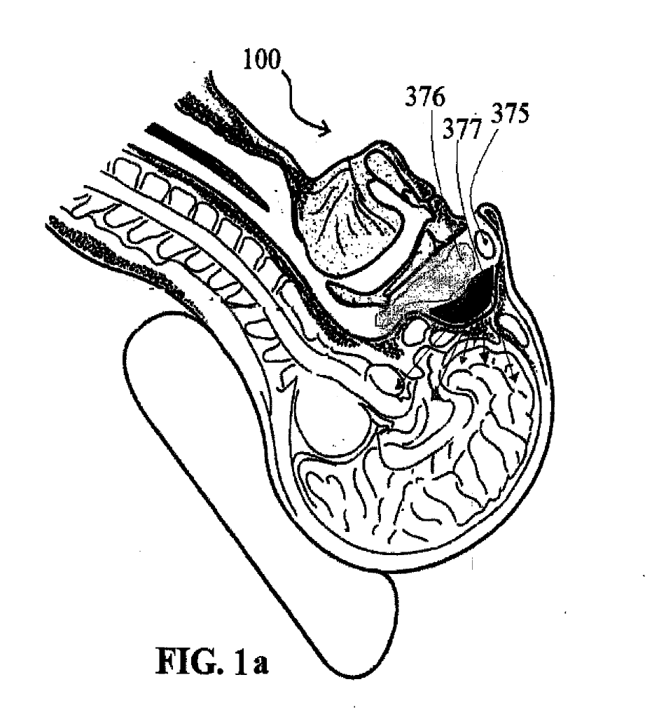

The term “olfactory region” (ORE) is same as “olfactory area of the nose” “olfactory mucosa” or “nasal olfactory area” includes olfactory mucosa, sphenopalatine ganglion and its branches, branches from the trigeminal nerve, sphenoid sinus and its 5 cranial nerves on the wall in the cavernous sinus, olfactory nerve fasciculi as they enter the olfactory bulb, anterior ethmoidal nerve, and the communicating blood vessels (CVVS) of this region to the CNS. It is located in the upper third of the medial and lateral wall of the nose (FIGS. 1, 2, 3, 6, 7) and covers the entire upper one third of the roof and walls of the nose, cribriform plate of the ethmoid bone, including sphenoid and ethmoid sinuses.

The term “olfactory mucosa” (OM) refers to the olfactory area in the upper part of the nose, which contains olfactory receptor bipolar neurons, that forms ±20 bundles of olfactory nerve fasciculi (FIGS. 1,2,3). The olfactory neuro-epithelium is the only area of the body in which an extension of CNS meets the external environment.

The terms “tumor necrosis factors,” (TNF), or “cytokines” refer to naturally occurring cytokines present in humans or mammals, which plays a key role in the inflammatory immune response and in the response to infection or autoimmune bodies.

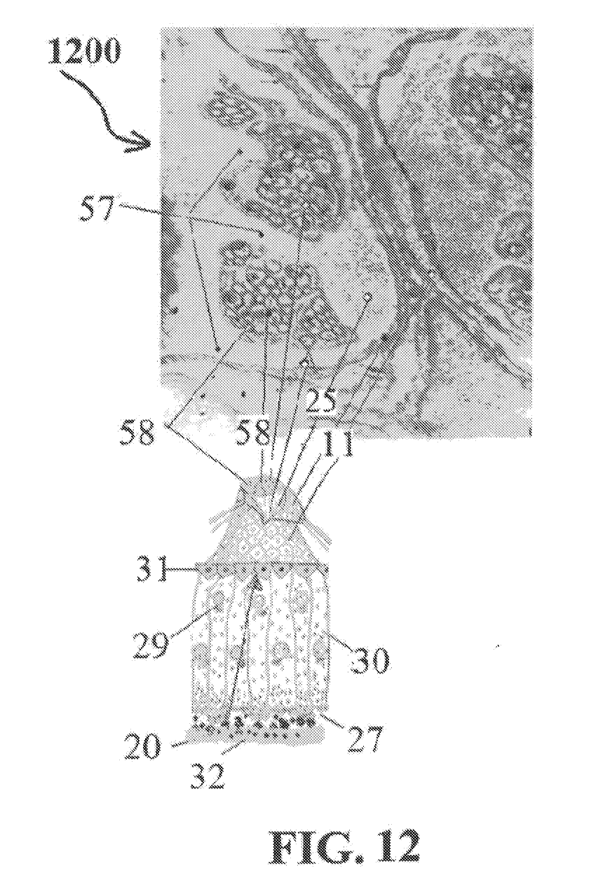

The term “perineural epithelium” (PE) refers to a histological structure of continuous flat squamous cell layers (FIGS. 12, 16, 17), completely surrounding the nerve fasciculi (axons bundles). Thus separating the axons from the tissue space around the nerve bundle and protecting them (Shantha T R and Bourne G H: Perineural epithelium: A new concept of its role in the integrity of the peripheral nervous system. Science 154:1464-1467 (1966).

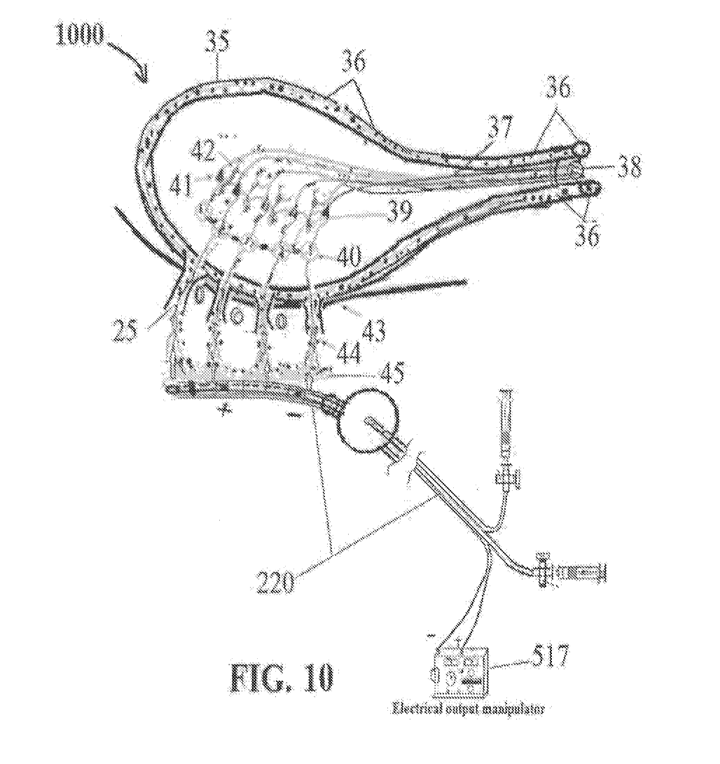

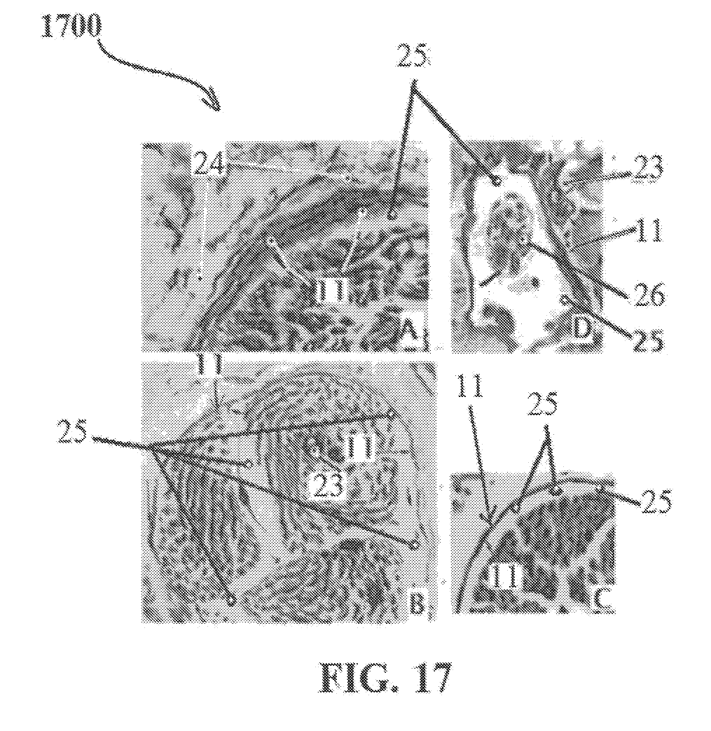

The term “sub perineural epithelial space” (sub PE) and “subperineural interstitial space” is the potential tissue space between the nerve bundles of axons (fasciculi) and below the perineural epithelium (FIGS. 10, 17) which conducts the bulk of the therapeutic agents from ORE and the other peripheral nerve fasciculi (Shantha T R and Bourne G H: The “Perineural Epithelium”: A new concept. Its role in the integrity of the peripheral nervous system. In Structure and Function of Nervous Tissues. Volume I. pp 379-458. (G H Bourne, Ed.). Academic Press, New York. 1969).

The terms “antibodies” and “immunoglobulins” mean the proteins produced by one class of lymphocytes (B cells) in response to specific exogenous foreign molecules (antigens, infections). They can be also be synthesized.

The term “monoclonal antibodies” (mAB) means the identical immunoglobulins that recognize a single antigen, derived from clones (identical copies) of a single line of B cell. This mAB can be a cytokine blocker, or a cytokine inhibitor, or as a cytokine antagonist.

A “composition” “compounded” or “medicament” encompasses a combination of an active agent or diluents, binder, stabilizer, buffer, salt, lipophilic solvent, preservative, adjuvant or the like, or a mixture of two or more of these substances. Carriers are preferably pharmaceutically acceptable.

The terms electrical “pulse,” “signal,” “impulse,” “drive,” and “force” gives the same meaning and are used interchangeably.

“Brain” and “CNS” signify the same structures, are used interchangeably, and may also include the brainstem and cerebellum.

The terms “treat,” “treating” and “treatment” “cure” “curtail” used herein, and unless otherwise specified, mean something which reduces, retards, or slows the progression and the severity of the disease using the invention and therapeutic agents described herein.

Abbreviations Used:

I. ACh=Acetylcholine

II. AChEIs=Acetyl-cholinesterase inhibitors

III. AD=Alzheimer's disease

IV. Aβ=Amyloid beta

V. BBB=blood brain barrier

VI. BV=Blood vessels

VII. CNS=Central nervous system.

VIII. CSF=Cerebrospinal fluid

IX. CSF=cerebrospinal fluid

X. CVO=circumventricular organ

XI. CVVS=valveless cranial-vertebral venous system

XII. CW=Circle of Willis blood vessels which supply the central nervous system

XIII. EC=entorhinal cortex

XIV. IGF-1=Insulin like growth factor

XV. IV=intravenous

XVI. mAB=monoclonal antibodies

XVII. mcg=micrograms, mg=milligrams

XVIII. ml=milliliter, mcg=micrograms, mg=milligrams

XIX. MS=Multiple sclerosis

XX. MW=Molecular weight

XXI. OM=Olfactory mucosa

XXII. ONA=olfactory nasal area

XXIII. ORE=Olfactory region includes olfactory mucosa, olfactory nerves, sphenoid sinus with cavernous sinus, trigeminal nerves, cranial nerves 1-6, and CVVS

XXIV. PE=Perineural epithelium

XXV. PNS=Peripheral nervous system

XXVI. ROS=reactive oxygen species

XXVII. SAS=Subarachnoid space

XXVIII. SPE=sub Perineural epithelial space

XXIX. SPG=sphenopalatine ganglion

XXX. TNF=Tumor necrosis factor

XXXI. VVS=Valveless Vertebral venous system of Batson

Detailed Description of the Diagrams Explaining the Invention to Treat Alzheimer's and how the Therapeutic Agents Reach the CNS to Cure or Curtail the Disease

These diagrams represent the present invention and describe how the therapeutic agents delivered to the CNS to treat CNS diseases including Alzheimer's, and deliver the electrical impulses to reach the site of pathology in the CNS to cure and curtail the affliction. While the preferred embodiment of the present invention has been described, it should be understood that various changes, adaptations, and modifications may be made thereto. It should be understood, therefore, that the invention is not limited to the details of the illustrated invention.

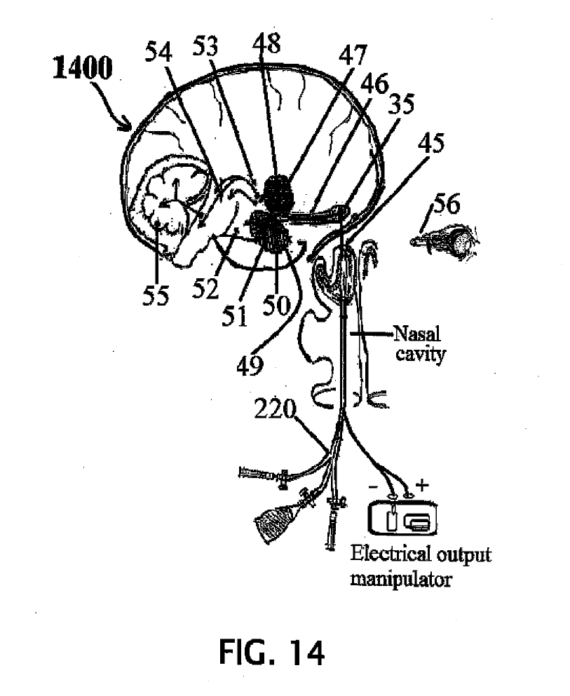

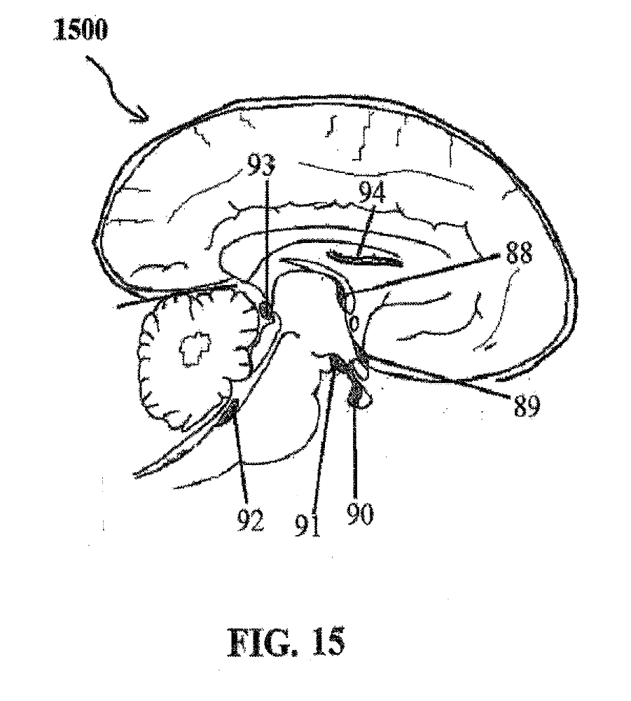

FIG. 1 is the diagram of the lateral and medial wall of the nasal cavity 100, presenting the area covered by the olfactory regions mucosa (ORE) all the way to the cribriform plate of the ethmoid bone 8. It illustrate the ORE with various nerve structures (shown in black surface with white lines) that therapeutic agents and electrical impulses come in contact with, then are conducted to the CNS to the brainstem, hippocampus, entorhinal cortex, thalamic, hypothalamic, cerebral cortical centers, cerebellum and other cortical neuropil (see FIG. 14) in the treatment of Alzheimer's disease. The olfactory tracts are connected to the entorhinal cortex (EC) located in the medial temporal lobe (area 28, and 34). The entorhinal cortex is one of the first areas affected in Alzheimer's disease. It functions as a center in a widespread network for memory and navigation-routing of impulses. The EC is the main interface between the hippocampus and neocortex. The EC-hippocampus system plays an important role in autobiographical/declarative/episodic memories and in particular spatial memories including memory formation, memory consolidation, and memory optimization. Therapeutic agents transported to this area as described directly to the brain as shown in this diagram (see also FIG. 15) and have a remarkable therapeutic effect on Alzheimer's patients and senile brain atrophy, as well as other neurodegenerative diseases.

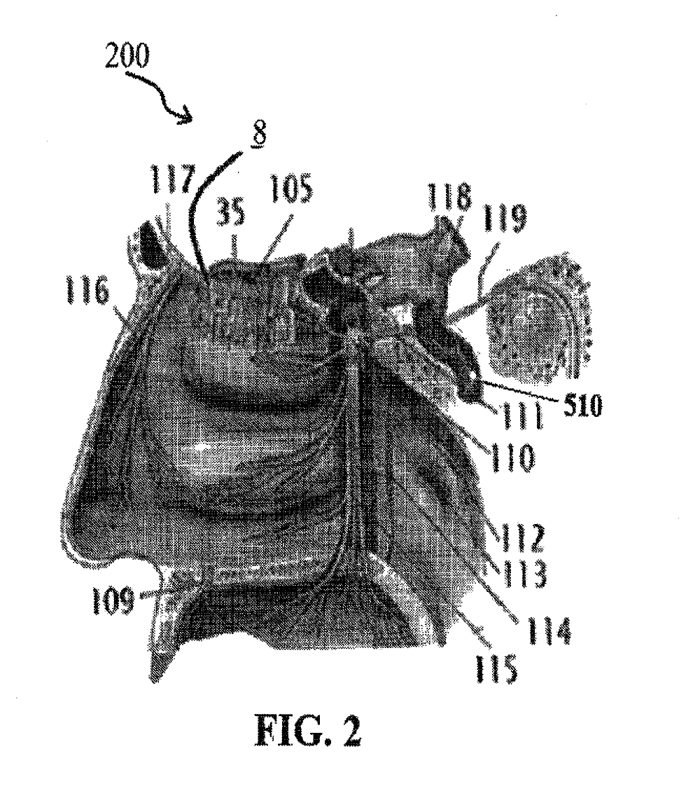

Note the olfactory mucosal region (ORE) with olfactory receptor and its nerve fasciculi 2, 5, cover extensive areas of the medial 3 and lateral 4 walls of the upper part of the nasal cavity, which is separate from the respiratory part of the nose (FIG. 1a). The olfactory nerves pass through the cribriform plate of the ethmoid bone 8 to the olfactory bulb. This region also contains the sphenopalatine ganglion (Pterygopalatine) 6 with its extensive central and peripheral nerve roots connecting branches (see FIG. 2 below). This ORE is also surrounded by anterior ethmoidal nerves 7 connected to the ophthalmic branch of the trigeminal nerves. The therapeutic agents delivered through this invention; transported to the CNS through the olfactory nerves. And also they are transported through trigeminal nerve branches 7 (CN V), III, IV, V (V1-2), VI to Cranial nerves 359; and sphenopalatine ganglion 6 that supply the upper third of nasal cavity close to the olfactory mucosa, pituitary gland 362 and sphenoid sinus 361 with 10 cranial nerves in its wall located in the cavernous sinus. The CSF in the SAS surrounding the olfactory bulb and olfactory nerves also conduct the therapeutic agents to the brain surface from short olfactory nerves in the treatment of Alzheimer's and other neurodegenerative diseases described.

FIG. 1a is the diagrammatic presentation 100a showing vestibule 375, respiratory nasal mucosa 376 with olfactory nerve and olfactory mucosa 377 of the lateral and medial walls of the olfactory mucosal nerve area of the nose (ORE). The arrows point to the spread of therapeutic agents from the ORE 377 to the CNS. Note to get the maximum delivery of therapeutic agents to ORE, the head should be extended as shown in the diagram and therapeutic agents delivered to the ORE 377 using the special delivery catheter described herein. Just spraying through the vestibule 375 will result in the delivery of therapeutic agents to the respiratory mucosa 376, where it is not effective for the treatment of Alzheimer's disease. The therapeutic agents' delivery catheter and lontophoresis device placed on the ORE 377 to treat Alzheimer's disease and other neurodegenerative diseases by passing the BBB.

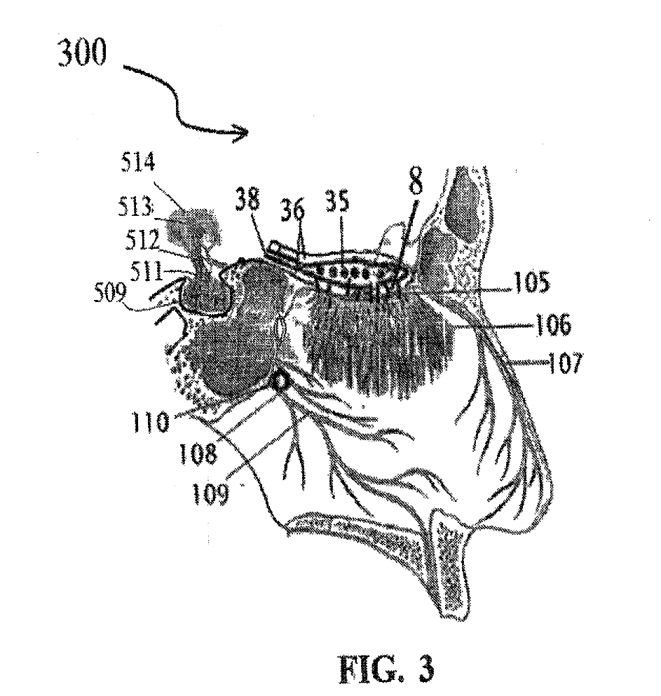

FIG. 2 is the diagram of the lateral wall of the nasal cavity 200 showing various nerve structures that the therapeutic agents and electrical current, used to create lontophoresis by this inventive device. The therapeutic agents described in this invention comes in contact with and are transported to the CNS through nerve fasciculi of the nerve structures located in the ORE, in the wall of the sphenoid sinus, and cranial vertebral venous plexus (CVVS). The subarachnoid space (SAS) and the cerebrospinal fluid (CSF) surrounding the olfactory nerve fasciculi and olfactory bulb as well as other cranial nerves described here also conduct the therapeutic agents to the surface of the brain. The delivery of therapeutic agents pass through the olfactory bulb 35 transported by the olfactory mucosa and olfactory nerves 105 passing through the cribriform plate of the ethmoid bone 8. The therapeutic agents passed on to the CNS through the trigeminal nerve 118, external nasal nerve 116, the anterior ethmoidal nerve 117; and from the sphenopalatine ganglion 110. From Sphenopalatine ganglion the therapeutic agents are conducted to the greater petrosal nerve 119, nerve of the pterygoid canal 111, pterygopalatine and pharyngeal nerve 112, lesser palatine nerve 114, greater palatine nerve 115, nasopalatine nerves 109 and parasympathetic's to the internal carotid artery 510. The sphenopalatine ganglion 112 neuronal center is located in just below the sphenoid sinus, posterior to the olfactory mucosa, behind the root of the nose (see FIG. 3) and receives the therapeutic agents delivered to ORE. The olfactory mucosa and its olfactory nerves 105 play a major role in delivering therapeutic agents in the treatment of Alzheimer's, in this invention, by bypassing or overcoming the BBB (diagram modified after Gray's Anatomy and lontophoresis). Iontophoresis and Electroporation facilitate the transfer of large MW therapeutic agents to the CNS in the herein described routes.

FIG. 3 is the diagram of the medial wall of the nasal cavity 300 and nerve structures located in the olfactory mucosal region (ORE). Various nerve structures on the medial wall of the nose conduct the therapeutic agents to treat Alzheimer's as this invention comes in contact, and transported to the CNS from the upper part of the nose from the ORE 106. The therapeutic agents of this invention transported through the olfactory nerves, through the cribriform plate of the ethmoid bone 8 to the olfactory bulb 35 from the olfactory mucosa 106. Olfactory nerves are the shortest of the cranial nerves; hence, it is easier for them to carry the therapeutic agents and the electrical impulses of lontophoresis to the olfactory bulb and its connections to the CNS without decay than for any other cranial nerves.

The axons and dendrites of the olfactory nerve tract transport and deliver therapeutic agents to the brain centers involved in Alzheimer's disease, but it is slow, the amount of therapeutic agents transported is minimal and there are many synaptic obstacles in the olfactory bulb (glomeruli) on the way to the final destination. Any therapeutic agents in the olfactory nerve (ten million olfactory nerve receptor cells) neuronal tubes and axoplasm held back at the rigid complex glomerular masses of synapses (1800 of them) in the olfactory bulb. Only when they bypass these synapses, can they travel further in the olfactory tracks connected to the CNS that is slow and minimal.

The therapeutic agents also pass through the trigeminal nerve branches 107 and sphenopalatine ganglion 110 that supply the nasal cavity through the anterior ethmoidal nerve 107, nasoplatine nerve 109, medial, posterior and superior nasal branches 108 and the sphenopalatine ganglion 110 and its branches to reach the circle of Willis to reach the brain stem cranial nerve nuclei. The therapeutic agents also pass from the sphenoid sinus to CN III, IV, V, VI, pituitary gland 509, rich vascular net work surrounding this gland 511 and pituitary stalk 512, pituitary hypothalamo-hypophysal tract 512, hypothalamic nuclei 513, and thalamic centers and then to the cortical radiation of the entire brain (Diagrams 2 and 3 Modified from Gray's Anatomy).

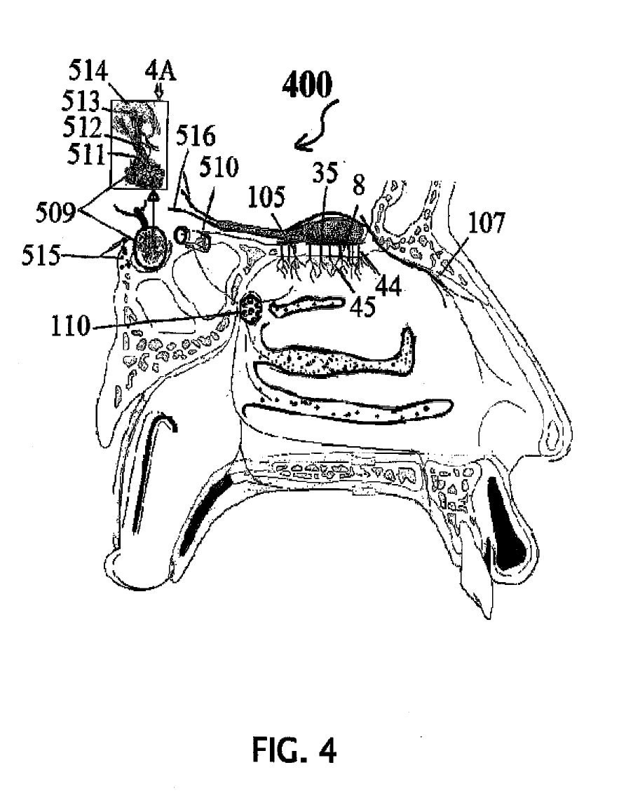

FIG. 4 is the lateral wall of the nasal cavity diagram 400 showing the nerve structure locations involved in the passage and transport of therapeutic agents, and transmission of electrical impulses to create Iontophoresis using this invention. The therapeutic agents conducted to the CNS from the olfactory mucosa 45, olfactory mucosal nerves 44, olfactory nerve fasciculi 105, olfactory bulb 35, and medial and lateral olfactory tracts 526. Therapeutic agents transported to the CNS from the sphenopalatine ganglion and its branches 110, parasympathetic supply from the sphenopalatine ganglion to Circle of Willis 510, pituitary gland 505, rich portal blood system of the pituitary gland 511, hypothalamo-hypophysal tract 512, hypothalamic nuclei 513, and thalamic radiation 514 (insert 4A). Note the presence of five cranial nerves 515 (CN III, IV, V₁₋₂, and VI) on each side of the cavernous sinus of the sphenoid sinus which are exposed to therapeutic agents delivered to cavernous sinus through CVVS and sphenoid sinus.

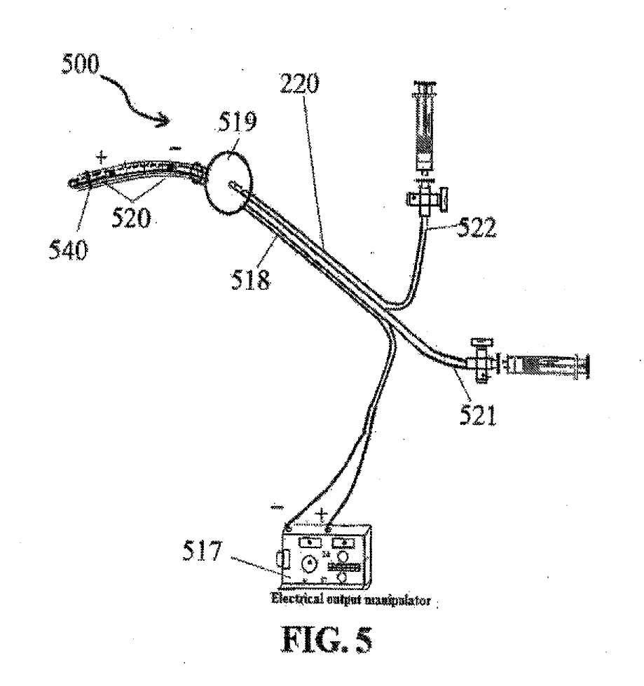

FIG. 5 is the diagrammatic presentation 500 of this inventive device 220 designed to stimulate the ORE to create lontophoresis, and deliver therapeutic agents to the ORE. It has electrical output manipulator 517 attached to the olfactory stimulator part 520 passing the conductive wires through the main body of the device 518. It has balloon 519, inflated while inserting and positioning the device in the ORE for easy positioning of the device. This balloon will prevent trauma to the delicate nasal mucosa as the device advanced to the ORE through the external nasal opening. The balloon connected to the inflating syringe 522. The balloon inflated with air or sterile liquid or gel and the size of the balloon adjusted according to the size of the patient's nose. The electrical current delivery part to create lontophoresis on the ORE of the device 520 also has pores to deliver therapeutic agents in the treatment of Alzheimer's and other diseases by delivering therapeutic agents from syringe 521. The tip of the inventive device provided with radio opaque marker 540 to identify the position of the catheter on the olfactory mucosa—sphenoid sinus after insertion and during insertion with radiographic examination.

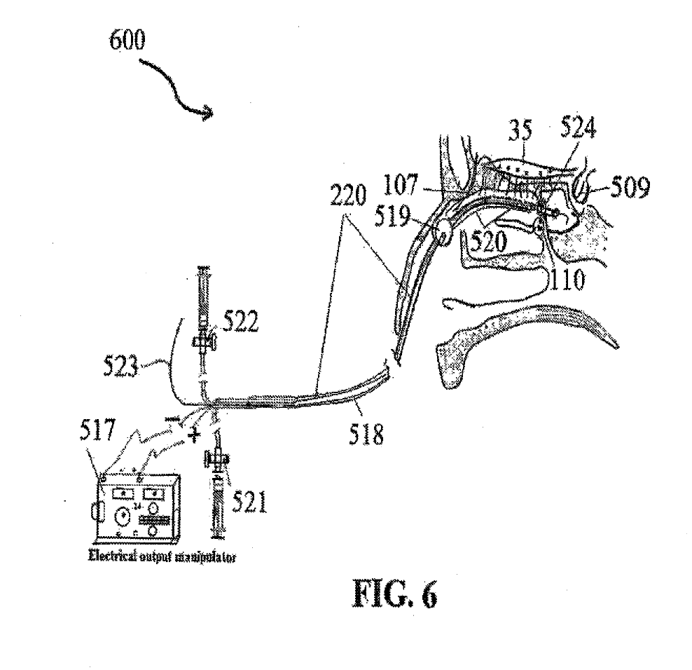

FIG. 6 is the drawing of the medial wall of the nose 600 showing various structures that are going to be stimulated by the nasal stimulator of by this invention device 220 to transmit the electrical pulses to the CNS and create electroporation and lontophoresis. Note the tip of the therapeutic agents and electrical impulses delivery device positioned in the sphenoid sinus through the ostium of the sphenoid sinus 524. This positioning between the sphenoid sinus 524 and the nasal balloon 519 will keep the lontophoresis stimulating part and the therapeutic agents delivery part of the device 520 located firmly in the desired location i.e. on the olfactory nerve mucosa close to the cribriform plate of the ethmoid bone as shown in the diagram. The electrical impulses delivered to create lontophoresis also pass (spillover effect) from this device to the sphenopalatine ganglion 110 and to the anterior ethmoidal nerve 107 and sphenoid sinus neural components. Injection port 521 utilized to pass the guide wire 523 to facilitate placement of this device with ease. The device insertion facilitated by the using flexible fiber optic nasal scope and guide wire 523. The electrical impulses are delivered through the electrical output manipulator 517 conducted through thin insulated conducting metal wires incorporated in the wall of the device.

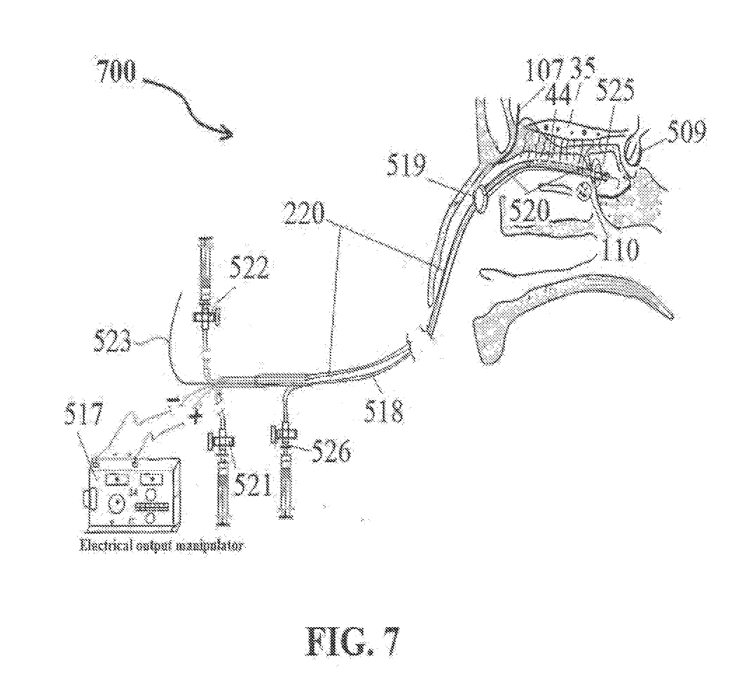

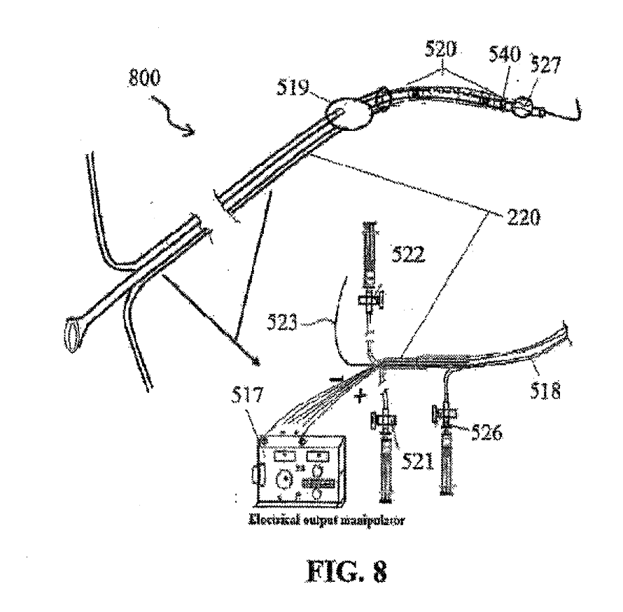

FIG. 7 is the view of diagram 700 of the present invention device 220 showing two balloons holding the therapeutic agents and electrical impulses delivering part of the device 520 in position between the sphenoid sinus with a balloon 525 and nasal balloon 519 without movement at the olfactory region for the treatment of Alzheimer's. The syringe 526 inflates the balloon in the sphenoid sinus 525 and the balloon in the nose 519 is inflated 522. The catheter and the balloon in the sphenoid sinus can incorporate with lontophoresis electrical embodiment to create lontophoresis in the wall of the sphenoid sinus. These inflated balloons hold the electrical impulses and therapeutic agent's delivery system on the olfactory mucosa (ORE) to the CNS in position, especially in patients who are difficult to control their movement. The syringe 521 delivers therapeutic agents to the ORE and sphenoid sinus. The diagram also shows the device 520 proximity to the anterior ethmoidal nerve 107, olfactory mucosa 44, olfactory bulb 35, pituitary gland 509, and the sphenopalatine ganglion 110. This also shows electrical impulses and therapeutic agent's spillover to these structures. The rest of the explanation is the same as FIGS. 5 and 6. FIG. 8 is the diagrammatic presentation 800 of the therapeutic agents and electrical impulses and therapeutic agent's delivery inventive device 220 to create lontophoresis and electroporation

This device incorporates olfactory nerve stimulator 520 and sphenoid sinus stimulator 527, which stimulates the five cranial nerves on the lateral wall of the sinus embedded in the cavernous sinus, the internal carotid artery (Circle of Willis) in each wall of the cavernous sinus located on the lateral walls of the sphenoid sinus to create lontophoresis. It also sends electrical impulses to pituitary gland to distribute the electric signals to the thalamic radiation and wake up the brain in those suffering from the Alzheimer's and other CNS diseases to create lontophoresis and neuronal electrical activation. The electrical impulses to create lontophoresis field deliverer terminals activated through the electrical output manipulator 517. The balloons 519 and 527 expanded by using the air or liquid by a tube in the interior connected through inflation stopcocks 522 and 526 connected by a tube to the inflation syringe located outside the nose. The syringe 521 delivers therapeutic agents through the pores located on the ORE area of the catheter to the olfactory mucosa and terminal pore in the catheter located inside the sphenoid sinus. The catheter provided with a guide wire 523 port to facilitate the positioning of the catheter on the ORE and inside the sphenoid sinus.

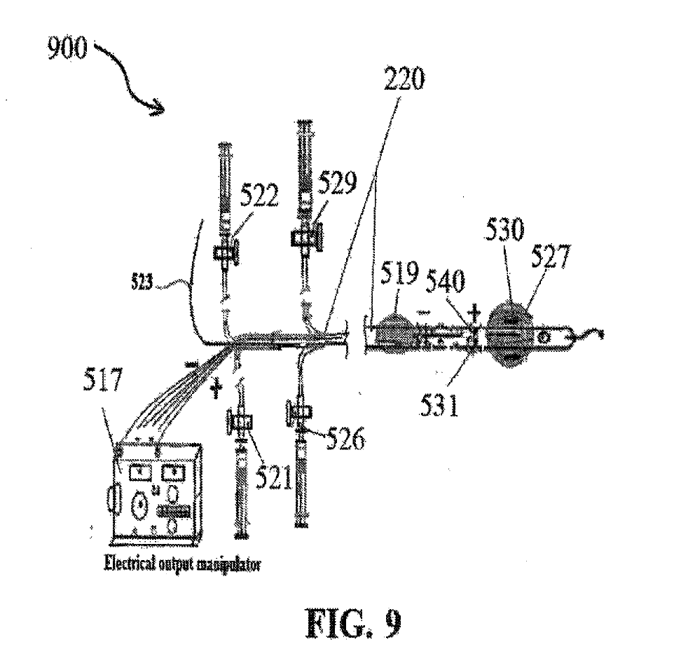

FIG. 9 is the diagrammatic presentation 900 of this invention, which incorporates many embodiments in the device 220. Many of the embodiments described in FIGS. 7, and 8. It shows the complete assembly of this inventive device to treat Alzheimer's diseases. It has two balloons 519, and 527. The balloon 527 part has the insertion body which is inserted through the nose through the sphenoid foramina and then into the hollow sphenoid sinus with the aid of a fiber optic nasal scope. The insertion body consists of two parts. One part is an inflatable outer membrane or balloon 527, which is adapted in size and flexibility to fit inside the sphenoid sinus cavity. The interior of this balloon 527 connected to an inflation tube, which in turn connected through an inflation stopcock and a tube to the inflation syringe 526. The inflation syringe 526 used to pump air or fluid through the inflation tube to the interior of the balloon 527 so it inflates filling the sphenoid sinus cavity during the operation of the apparatus. An infusion tube is also connected to the interior of the balloon 527 and is used to pump fluid at ambient, elevated, or low temperatures through the infusion tube and to the interior of the balloon during the operation of the apparatus. A device for heating or cooling the fluid to be pumped into the interior of the balloon 527 may also be included in the apparatus (not shown in the diagram). The balloon 527 is provided with multiple electrical leads on the exterior of the balloon as shown on the balloon. These electrical leads are connected by electrical connectors to an electrical output manipulator 517. Electrical stimulus (electrical impulses) provided through the electrical leads to stimulate and create Iontophoresis fields to deliver large MW therapeutic agents to the CNS bypassing blood-brain barrier.

A catheter placed on the surface or the center of the balloon with a suitable tube to administer drugs or other fluids directly to the sphenoid sinus cavity from the syringe 529, as desired for treatment of Alzheimer's and CNS diseases besides delivering the electrical impulses. The therapeutic agents are infused so that they are absorbed by the central nervous system directly across the sphenoid sinus walls into the perforating cranial-upper cervical valveless venous system vessels (CVVS), which empty into the cavernous sinus plexus and circulate in the BV of the CNS and then to neuropil. The therapeutic agents also pass on to sub Perineural epithelial space 25 and then into SAS and CSF 36 through the 5 cranial nerves that traverse through the cavernous sinus. This method allows us to use a small dosage of drugs instead of using large dosages systematically to avoid any therapeutic agent's adverse effects. The antibiotics and anticoagulants impregnated into the surface of the device 220 and balloons of the sphenoid sinus cavity to prevent clotting and infection. The tip of the inventive device provided with radio opaque marker 540 to identify the position of the catheter tip in the sphenoid sinus after insertion and during use with radiographic examination.

All of the tubes and connectors to the balloon 527 are assembled in a connector assembly of the device 220. The inner portion of this connector assembly constitutes part of the insertion body. This assembly needs to be small in diameter and flexible for easy insertion through the nose and into the sphenoid sinus cavity ostium.

A temperature sensor wire is connected to a temperature sensor and indicator outside located in the electrical output manipulators. The temperature sensor wire is connected to sensors (not shown) in the balloon 527 to determine the temperature of the balloon surface and the structures in the immediate vicinity of it. This fluid within the balloon may be heated to 42°-44° C. or higher or cooled if so desired to stimulate or decrease the output of pituitary hormones, including growth hormone from the pituitary gland. Other means such as a device embodying the Peltier 530 effect can be used to heat or cool the outer surface of the balloon. Heating will enhance the conduction of electrical impulses and facilitate the stimulation of the pituitary gland and other surrounding nerve structure. The cooling will have the reverse effect.

FIG. 10 is the diagrammatic presentation 1000 of the longitudinal section of the olfactory bulb 35 and the olfactory mucosa showing the route therapeutic agents take and electrical impulses transmission to create Iontophoresis on ORE to transport insulin and other therapeutic agents from the ORE in the treatment of Alzheimer's inventive method delivered through the device 220. The therapeutic agents pass through the olfactory nerves (shortest cranial nerve- 3/16 to ⅜ of inch long) from the olfactory mucosa 45 and transported through the subperineural epithelial space 25 and olfactory axons to the olfactory bulb 35 in this invention to treat AD. The therapeutic agents mainly transported to CNS by sub arachnoid space (SAS) 36 after passing through the olfactory nerve fasciculi surrounded by perineural epithelium 25 with CSF surrounding them. The SAS surrounding the olfactory bulb with its CSF is directly connected to the sub perineural epithelial space surrounding the olfactory nerve fasciculi 25 and transmits the therapeutic agents facilitated by the lontophoresis of the ORE [Shantha et al: Z. Zellforsch. 103, 291-319 (1970). J National Cancer Inst 35(1):153-165 (1965). Expt Cell Res 40:292-300 (1965). Science 154:1464-1467 (1966). Nature 199, 4893:577-579 (1963). Nature, 209:1260 (1966). Histochemie 10:224-229 (1967). Structure and Function of Nervous Tissues. Academic press, 1969, Volume I. pp 379-458).

The therapeutic agents pass from receptor cells 44 and transported through the axons, olfactory nerve fasciculi, retrograde through the cribriform plate of the ethmoid bone 43 to the olfactory bulb 35. From the olfactory receptor cell axons 45, the therapeutic agents travel through the olfactory glomeruli 40 to periglomerular cells 39, mitral cells 41, and granule cells 42, to olfactory tract 37, and reach the CNS 38 and then to entorihinal cortex. Such a transport mechanism takes time and not much quantity of therapeutic agents transported to the CNS due to blockade at the massive multiple synaptic glomeruli in the olfactory bulb. It is the sub Perineural epithelial, and nerve fascicular interstitial spaces, around the axons above the endoneurium conducts the majority of the therapeutic agents to the CNS (Shantha IBID).

This diagram shows that the inventive device placed on the olfactory nerve embedded olfactory mucosa to stimulate the olfactory mucosa to create pores and electromotive force in the olfactory mucosa membrane by lontophoresis and electroporation currents delivered through the electrical output manipulator 517 for delivery of large molecular weight therapeutic agents.

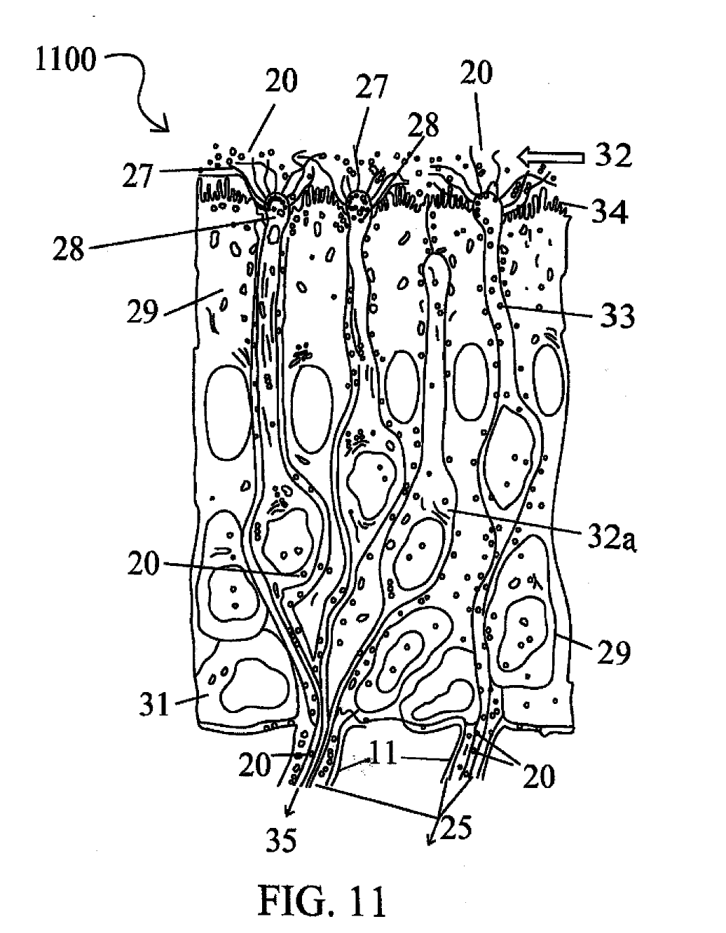

FIG. 11 is the drawing of the section of the olfactory mucosa 1100, labeled with names of structures with numbering to demonstrate the histology of the olfactory mucosa; and how the therapeutic agents transported to the CNS by passing the BBB. It is showing the route taken by the insulin and various compounded therapeutic agents described in this invention and their path of transfer through the olfactory nerve (±20 nerve fasciculi) to olfactory bulb and CSF in SAS of the CNS to treat Alzheimer's disease. The diagram shows; how the insulin, bexarotene, ketamine, monoclonal antibodies, IGF-1, and cholinesterase inhibitor therapeutic agents used in this invention get attached to the mucous film 32. Then they are entangled in olfactory cilia 27 of the olfactory cells and microvillus 34 of the supporting cells 29. Then they are transported to through the olfactory axons 20, and Perineural epithelium 11 and sub Perineural epithelial, and nerve fascicular interstitial spaces 25 to the olfactory bulb 35 and the SAS surrounding the olfactory bulb containing CSF (see FIG. 10). Note the space created by dying olfactory cell 33, developing receptor cells 32a, and their dendritic bulb 28 make sieve like holes in the olfactory mucosa that facilitated the passage of therapeutic agents from the olfactory mucosa to the olfactory bulb. These holes in the olfactory mucosa easily transmit the insulin and other large molecular weight therapeutic agents 20 described herein to the olfactory bulb 35 and the rest of the CNS. The basal cells 31 transfer the insulin and therapeutic agents from the surface mucosa 20 to the capillary space around the axons and to the sub perineural space below the perineural epithelium 25. There are hundreds of olfactory cells 33 dying at different locations of olfactory mucosa in a given time. This creates a space between the olfactory cells and supporting cells which makes the olfactory membrane porous like a sieve creating a route for the easy transport of insulin and other therapeutic agents from the olfactory mucosal surface 20 used in our invention. Furthermore, the creation of Iontophoresis and electroporation by this device facilitates easy and rapid transfer of large molecular weight therapeutic agents through the ORE. The insulin, bexarotene, ketamine, monoclonal antibodies, IGF-1, and cholinesterase inhibitor therapeutic agents transmitted to the CNS through the axons of olfactory bulb 35 (hardly any). The sub Perineural epithelial, and nerve fascicular interstitial spaces 25 (major route of therapeutic agents transport) surrounding the olfactory axon bundle (fascicule—see FIG. 12), where they enter the olfactory bulb through the cribriform plate of the ethmoid bone (Shantha T. R. and Yasuo Nakajima. Yerkes Regional Primate Research Center, Emory University, Atlanta, Ga.: Histological and Histochemical Studies on the Rhesus Monkey (Macaca Mulatta) Olfactory Mucosa. Z. Zellforsch. 103, 291-319, 1970).

FIG. 12 is the drawing of the section of the olfactory mucosa and actual electron micrograph of olfactory nerve fasciculi 1200. The diagrams show the route taken by the insulin, bexarotene, ketamine, monoclonal antibodies, IGF-1, and cholinesterase inhibitor therapeutic agents and their path of transfer to the through the olfactory nerve 58 (±20 nerve fasciculi) to olfactory bulb SAS, and CSF of the CNS to treat Alzheimer's disease using our inventive device and therapeutic agents. It shows how the therapeutic agents get attached to the mucous film 32 are entangled in olfactory cilia of the olfactory cells 29 and microvilli 27 of the supporting cells 29, and transported to through the olfactory axons 58, and sub Perineural epithelial space spaces 25,57 to the olfactory bulb and the SAS surrounding the olfactory bulb containing CSF (FIG. 10). The basal cells 31 transfer the therapeutic agents from the surface mucosa 20 to the capillary space around the axons and to the sub perineural space below the perineural epithelium 25. There are hundreds of olfactory cells 29 dying and replaced at different locations of olfactory mucosa. This creates a space between the olfactory cells and supporting cells which makes the olfactory membrane porous like a sieve creating a route for the easy transport of insulin and other therapeutic agents from the olfactory mucosal surface 20 used in our invention. The therapeutic agents; insulin, bexarotene, ketamine, monoclonal antibodies, IGF-1, and cholinesterase inhibitor described in this invention are transported to the CNS through the axons 58 of olfactory mucosa (hardly any). Most of these therapeutic agents are transported through sub Perineural epithelial, and nerve fascicular interstitial spaces, 25 (major route of transport) surrounding the olfactory axon bundle where they enter the olfactory bulb through the cribriform plate of the ethmoid bone (Shantha T. R. and Yasuo Nakajima. Yerkes Regional Primate Research Center, Emory University, Atlanta, Ga.: Histological and Histochemical Studies on the Rhesus Monkey (Macaca Mulatta) Olfactory Mucosa. Z. Zellforsch. 103, 291-319, 1970).

The ducts of the Bowman's glad open on the surface of the olfactory mucosa, and transport the therapeutic agents to the glandular system located in the lamina propria. From the lamina propria, the therapeutic agents transported to the olfactory nerve sub Perineural epithelial, and nerve fascicular interstitial spaces 57, and to the cranial vertebral venous plexus, and then find their way to the CNS. Iontophoresis will deliver large therapeutic agents to the openings of these glands, which stay for longer periods of time and transported slowly through the duct system of the gland. Therefore, the therapeutic effect continues even after cessation of use of this device and therapeutic agents.

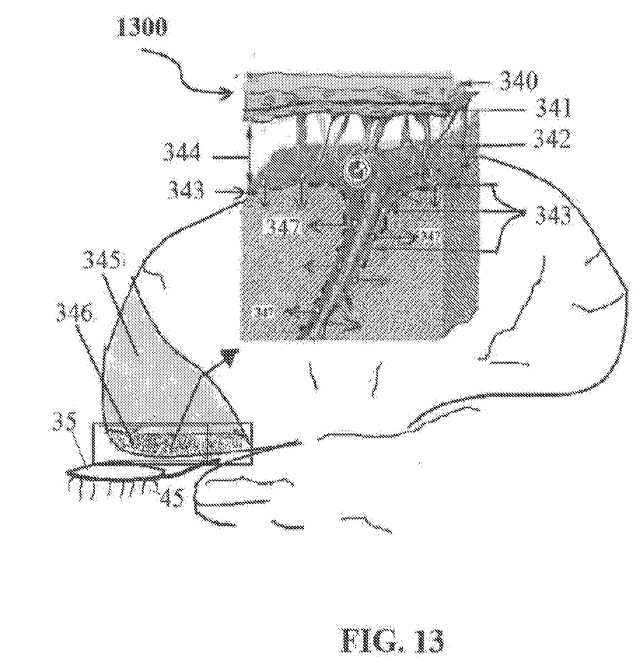

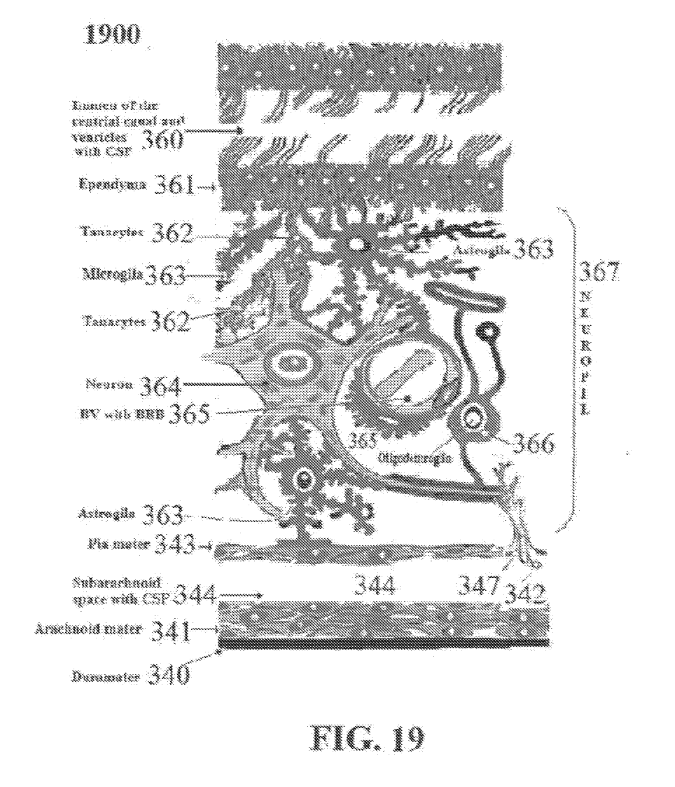

FIG. 13 is the diagram 1300 of the Virchow-Robin space in the central nervous system which plays a role in the transport of therapeutic agents from the SAS CSF to neuropile. It shows the olfactory bulb 35 and olfactory mucosa 45 which delivers the insulin, bexarotene, ketamine, monoclonal antibodies, IGF-1, and cholinesterase inhibitor therapeutic agents to the SAS 344 into the CSF and then to cortex of the CNS (Arrows). The CSF in SAS is the fluid media which spreads the ORE delivered therapeutic agents. Once in the CSF, therapeutic agents enter the neuropile throgh the pia mater, Virchow-Robine space 347, pial covering 343, CVO, CVVS, and through the penetrating blood vessels in the neuropil. This digram shows that the dura mater 340 located immediately below the skull bones has no role in delivery of therapeutic agents. The arachnoid mater 341, sub arachnoid space 344 with CSF, pia mater 343 extending on the blood vessel deep in the cortical part of the brain, brain stem, and spinal cord to form the Virchow-Robin space 347 play a key role in transfer of therapeutic agents from SAS. The CSF permeates this space down into surface of the CNS (arrows), and lets the therapeutic agents percolate and permeate all through the neuropil and back to central canal of the spinal cord and ventricles of the brain and vice versa (see FIG. 19). The therapeutic agents also enter the brain neuropil through the blood vessel and pial absorption of therapeutic agents from the CSF of the SAS as they pass through these spaces. The therapeutic agents absorbed through the intracerebral capillaries are unable to deliver much of the therapeutic agents due to the presence of BBB (98% blockage) unless it is breached artificially. Some of the insulin, bexarotene, ketamine, monoclonal antibodies, IGF-1, and cholinesterase inhibitor therapeutic agents of our invention enter the delicate BV as they pass through the SAS to enter the brain. This diagram also shows the prefrontal 345 and pre supraorbital 346 cortx which is located close to the temporal lobes, and the olfactory bulb where the therapeutic agents of our invention are delivered in addition.

The majority of the CSF in the brain is located in the pontine cistern and cisterna magna and the rest of CSF surrounds a capillary thin SAS covering cerebral hemispereres, cerbellum and spila cord as well as the optic nerve. The various known therapeutic agents, as well as other pharmaceutical, biochemical, nurticeuticals, and biological agents or compounds with insulin, bexarotene, ketamine, monoclonal antibodies, IGF-1, and cholinesterase inhibitor therapeutic agents in our invetion pass hrough the SAS in front of the brain and brain stem CSF from the olfactory bulb 35 and olfactory mucosa 45, trigeminal pathwasys, sphenopalatine ganglion connections, and CVVC. Hence, the Virchow-Robine space 347 delivers the insulin, bexarotene, ketamine, monoclonal antibodies, IGF-1, and cholinesterase inhibitor therapeutic agents to these regions rapidly. It is the delivery of these therapeutic agents through the Virchow-Robin space 347 and pial membrane 343 deep into the surface of the CNS that is responsible for the therapeutic effect to cure and/or curtail Alzheimer's disease. That is how the insulin and the other therapeutic agents described in this invention from the ORE reach the CNS and exert their therapeutic effect (diagram modified from Grays Anatomy).

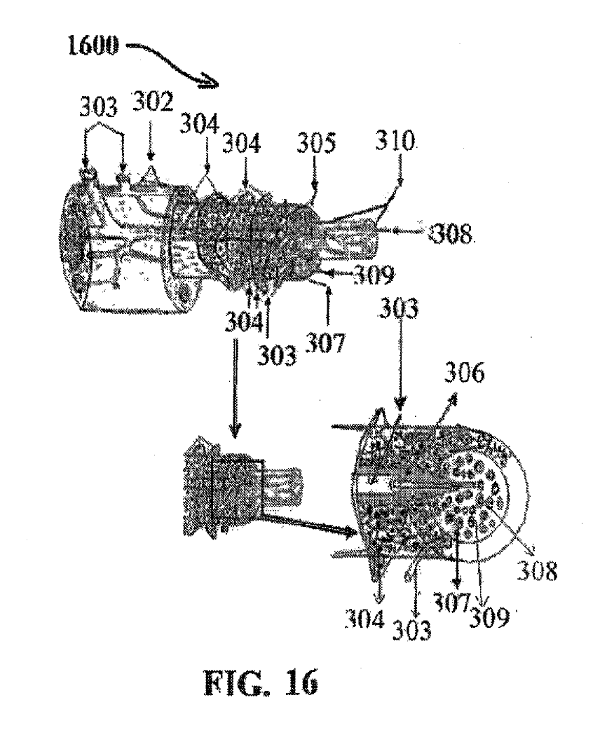

Virchow-Robin spaces 347, also known as enlarged perivascular spaces are spaces (often only potential) that surround perforating blood vessels of the cortex and spinal cord for a short distance as they enter the brain 347, spinal cord, and peripheral nerves (see FIG. 16 #306). Their wall formed by prolongations of the pia mater in the CNS and perineural epithelium in the peripheral nervous system (one cell thick). The spaces function as pathways for the transfer of insulin, bexarotene, ketamine, monoclonal antibodies, IGF-1, and cholinesterase inhibitor therapeutic agents and other therapeutic agents to enter deep into the surface of the brain and drain interstitial fluid from the neuropil. The Virchow-Robin space in the CNS 347 and the subpial space are separated by a single layer of pia mater 343 from the subarachnoid space, which can transport the therapeutic agents to the neuropile from the SAS through the permeation of CSF.

The brain and the spinal cord bathed in cerebrospinal fluid (CSF) 344, which carries the therapeutic agents of our invention inside the brain. CSF secreted by the choroid plexus in lateral, III^{rd}, and IV^{Th} ventricles in the brain via the weeping or transmission of tissue fluid by the brain and BV into the ventricles. The choroid plexus also has weak BBB compared to the intra cerebral capillaries. From here, the CSF percolates down the cerebral cortex ventricles, brain stem, and the spinal cord in the space between the pia and arachnoid mater (SAS). The overflowing CSF empties into the blood of the venous sinuses via the arachnoid villi in the sagittal sinuses, intracranial vascular sinuses, optic nerve and spinal nerve root arachnoid villi (Shantha T R and Evans J A: Arachnoid Villi in the Spinal Cord, and Their Relationship to Epidural Anesthesia. Anesthesiology 37:543-557, 1972. Shantha T R and Bourne G H: Arachnoid villi in the optic nerve of man and monkey. Expt Eye Res 3:31-35 (1964)) and, thereby potentially delivering therapeutic agents transported to the SAS and neuropil via he ORE to the central nervous system.

The average human has 100-150 ml of CSF, 20% of which is located in the brain ventricles, 20% in the subarachnoid space (above the pia), and 60% in the lumbar cisterns of the spinal cords. The choroid plexus produces approximately 450 ml of CSF per day, about 21 ml in adults and 10 ml in children per hour, enough to replace the CSF contents 3 to 4 times a day. CSF flows from the choroid plexus into the lateral ventricles, through the interventricular foramen of Monroe, into the third ventricles, out the cerebral aqueduct of Sylvius, and into the fourth ventricle. It then moves out from the fourth ventricle through the foramen of Lushka (two lateral pores) and Magendie (one central pore) into the pontine cisterns and cisterna magna (the spaces below and above the brainstem and upper cervical spinal cord).

Because the CSF exchanges substances freely with the interstitial fluid that surrounds the brain's neurons and glial cells (neuropile), these two extracellular fluids are likely to have similar compositions though there is a gradient favoring passage of substances in the extracellular fluid from the brain to the CSF. The CSF is constantly circulating around the brain, spinal cord, and interior of the brain through ventricles and central canal of the spinal cord, carrying substances in and out of the CNS (FIG. 19). Hence, the CSF can act as a continuous everlasting sprinkler system delivering neurotrophic and therapeutic agents to the brain, spinal cord and their extensions as far away as the peripheral nervous system (PNS) including the sensory and motor end organs. The relatively harmless accessibility of the CSF compartments to the CNS and PNS made it a desirable route for delivery of therapeutic agents to the extracellular compartments of the brain parenchyma and PNS. The CSF plays an important role related to drug penetration, permeation, distribution, and clearance in the treatment of AD and other neurological diseases.

In the human, the dura 340 is thick, impermeable, and opaque; whereas, the arachnoid 341 and pia 343 is thin, somewhat permeable, and translucent. The CSF occupies the subarachnoid space 344. When a person is lying down, the CSF pressure is 4-16 mm Hg; the pressure increases as the person sits up, since the pressure reflects the column of fluid. The CSF pressure influenced by venous pressure and typically pulsates with breathing and heartbeats. This CSF pulsation movement helps to dissipate the therapeutic agents delivered to SAS, and propel them to the surface of the CNS. The average CSF movement in the posterior spinal subarachnoid space is towards the tail (caudad) while the average CSF movement in the anterior spinal SAS space and central canal tend to be toward the head (Cephalad), which might be due to the effect of the heart's and lungs' pulsatile force and denticulate ligament of the spinal cord. That is why the therapeutic agents from the ORE come in contact with the fore part of the cerebral cortex, temporal lobes, front of the brain stem (CSF in cerebro-pontine cistern); and stay in contact with these areas of the brain and entorihnal cortex a longer period of time in our method of the treatment of Alzheimer's disease using insulin, bexarotene, ketamine, monoclonal antibodies, IGF-1, and cholinesterase inhibitor therapeutic agents. Therefore, intrathecally and ORE administered drugs in the posterior subarachnoid space and cisterna magna move downward towards the caudal (tail ward) spinal cord and then back towards the rostral (cephalad, head ward) end of the cord, brain stem and the rest of the cerebral cortex and cerebellum. The higher the level of intrathecal administration (for example cisterna magna, upper cervical SAS), the faster and higher the concentration of therapeutic agents delivered to the brain.

FIG. 14 is the diagrammatic presentation 1400 and the therapeutic agent's delivery device 220 to the ORE. It show the section of the olfactory mucosa 45 lining of the nose close to the cribriform plate of the ethmoid bone and the olfactory bulb 35 within the cranium situated immediately above cribriform plate of the ethmoid bone and the olfactory mucosa 45. The diagram is showing the passage and transport of therapeutic agents delivered through the device 220 and route taken by the therapeutic agents deposited at the olfactory region of the nose (ORE) in this invention to treat Alzheimer's and other neurological diseases. The therapeutic agents from the olfactory mucosa 45 are transported to the olfactory bulb 35 to the subarachnoid space (SAS) to the cerebrospinal fluid (CSF) and then to the cerebro-pontine cistern and then to various centers of the CNS. The therapeutic agents spread to the SAS CSF, olfactory tract 46, Entorhinal cortex, to prefrontal cortex 47, medial olfactory area 48, to temporal lobes 50, to lateral olfactory area 51, hippocampus 52, hypothalamus 53, brain stem nuclei 54, to cerebellum 55 and help in curing or curtailing Alzheimer's and other neurodegenerative diseases. The arrows show the extensive area where the therapeutic agents spread from the ORE to the CNS.

FIG. 15 is the drawing of the location of the circumventricular organs 1500 that play a role in the passage of the therapeutic agents to CNS due to CSF and vascular spread to treat Alzheimer's disease by insulin, bexarotene, ketamine, monoclonal antibodies, IGF-1, and cholinesterase inhibitor therapeutic agents from the ORE, and CVVS. There are several areas of the brain known as “circumventricular organs” (CVO) where the BBB is weak and allows therapeutic, pharmaceutical, biochemical, and biological agents or compounds to cross into the brain and CSF freely with the least impediment compared to the blood vessels with BBB within the neuropile of the CNS. The circumventricular organs are where the therapeutic agents also enter the CNS through the CSF and neuropile. Such Circumventricular organs include the Pineal gland 93 that secretes melatonin and is associated with circadian rhythms; and Neurohypophysis 90 (posterior pituitary) that produces oxytocin and vasopressin into the blood to maintain BP and the urine output. Area postrema 92, a chemo sensitive vomiting center in the fourth ventricle of the brain stem, and Subfornical organ 88 are involved in the regulation of body fluids. Vascular organ of the lamina terminalis 89, a chemosensory area, detects peptides and other molecules. Median eminence 91 regulates the anterior pituitary through release of neurohormones. Note how close the areas 89,90, and 91 are located to olfactory tracts and the crebro-pontine CSF cistern, which can easily transfer the therapeutic agents of this invention to these areas. To the circumventricular organs, I would add choroid plexus 94, Ependymal lining of the ventricles and central canal, arachnoid villi, pia mater of the brain and spinal cord, and the emerging nerve roots of the CNS and Spinal cord (Shantha T R and Evans J A: Arachnoid Villi in the Spinal Cord, and Their Relationship to Epidural Anesthesia. Anesthesiology 37:543-557,1972. Shantha T R and Bourne G H: Arachnoid villi in the optic nerve of man and monkey. Expt Eye Res 3:31-35 (1964). Nakajima Y, Shantha T R and Bourne G H: Histological and Histochemical studies on the subfornical organ of the squirrel monkey. Histochemie 14:149-160 (1968). Manocha and Shantha. Enzyme Histochemistry of the Nervous System (Macaca Mulatta, 1970, Academic Press, 18-305).

FIG. 16 is the drawing of the peripheral nerve fasciculi 1600 showing the structure of the peripheral nerve (PNS) fasciculi (trigeminal and sphenopalatine ganglion afferents and efferent fasciculi, other spinal PNS and cranial nerves). It shows coverings, blood vessels 303, perineural and perineural epithelial connective tissue 302, multiple layers of perineural epithelium 304 surrounding each nerve fasciculi, and blood vessel traversing between the layers of Perineural epithelium 305 to form Virchow-Robin space 306 in peripheral nerve fasciculi. The Virchow Robin space surrounds the BV 303 as they enter the nerve fasciculi for a very short distance 306. Note the distinct sub perineural epithelial space below the perineural epithelium covering of the nerve fasciculi 307 which communicates with the interstitial space around each axon 309 (sub Perineural epithelial, and nerve fascicular interstitial spaces) surrounded by the scanty delicate endoneurium and thick myelin sheath. Each axon surrounded by minimal endoneurium 308.

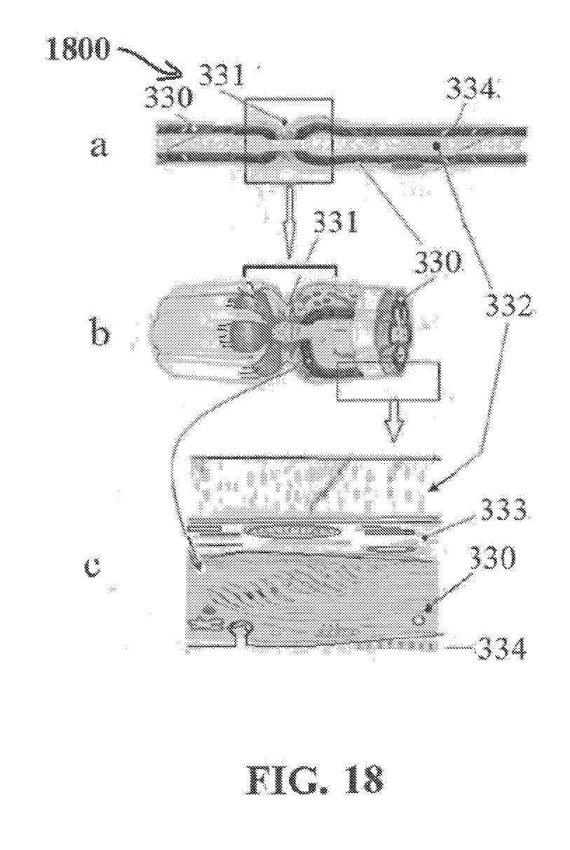

The mechanism of transfer of the insulin, bexarotene, ketamine, monoclonal antibodies, IGF-1, and cholinesterase inhibitor therapeutic agents administered in our invention to treat Alzheimer's disease has to enter the inside the nerve fasciculi to be transported retrograde to the CNS by the axonal nerve fasciculi. The therapeutic agents have to pass through the nerve fasciculi connective tissue (epineurium), perineural epithelium, Virchow-Robin space, and sub perineural epithelial space, then pass on to sub perineural epithelial space and interstitial space between axons. From these spaces, the therapeutic agents used to treat Alzheimer's disease enter the node of Ranvier, then enter the axoplasm, and transported retrograde by axoplasm (see FIG. 18) which is minimal. Most of the therapeutic agents transported through the sub perineural epithelial and sub perinueral interstitial space in the nerve fasciculi. This is the important route in transporting therapeutic agents to the SAS and CSF of the CNS. These are the major routes of transport of therapeutic agents to CNS and the axonal transport plays only a minor role, though it plays a major role in retrograde transport of rabies virus (Baer G M, Shantha T R and Bourne G H: Studies on the pathogenesis of fixed rabies virus in rats. J Bulletin of the World Health Organization 33:783-794 (1965). Baer G M, Shantha T R and Bourne G H: The pathogenesis of street rabies virus in rats. Bull World Hlth Org, 38(1):119-125 (1968)). From here, the insulin, bexarotene, ketamine, monoclonal antibodies, IGF-1, and cholinesterase inhibitor therapeutic agents are distributed to the surface of the brain from where they enter the neuropil to treat Alzheimer's and other neurodegenerative disease. That is how the therapeutic agents of our invention spread to the CNS from the trigeminal nerve branches and sphenopalatine ganglion of the ORE, cranial nerves in the walls of sphenoid sinus (after Shantha T. R, Virchow-Robin space in the peripheral nerves, 1992, ASRA March-April Supplement).

Once the therapeutic agents are inside the nerve fasciculi around the edoneural surroundings, they can enter the axons at two sites. 1. They can enter the unmyelinated small axons surrounded by Schwann cells without myelin (mostly in autonomic nerve fibers), but not through the myelin of the most peripheral nerve axons in the nerve fasciculi, and 2. The therapeutic agents can enter the axoplasm only through the Node of Ranvier in a thickly myelinated axon (most of the axons in the peripheral nerve fasciculi of PNS) which is a metabolically active site on the axons, lacking an insulating permeability resistant myelin sheath. The myelin sheath surrounding the axon is almost impermeable to most of the therapeutic agents.

FIG. 17 is the Histological Section of nerve fasciculi A, B, and C showing strong lactic dehydrogenase activity in the perineural epithelium cells (arrows) whereas the perineural connective tissue shows negligible activity 24. The axons show strong positive activity whereas the myelin sheath shows negligible activity. The Schwann cell cytoplasm also shows positive activity for this test. Note that the nerve fasciculi are surrounded by perineural epithelium 11 and form the sub perineural epithelial space below it 25. Some of the perineural epithelium cells split the nerve fasciculi also into smaller compartments. The sub perineural epithelial space surrounds the nerve bundles and communicates with the interstitial space surrounding the axons with their endoneurium surrounds (×275). FIG. 12 B. is the Rat trigeminal nerve section showing alkaline phosphatase activity in perineural epithelium cells (long arrows) 11. Note the peeling off the innermost layer of these cells (short arrows) 11 which enter to form the perineural septa, thus subdividing the large nerve fasciculus. The sub perineural epithelial space 25 is formed around the nerve fasciculi by the perineural epithelial sheaths (×275). FIG. 12 C. is the cross section of the trigeminal nerve showing strongly ATPase-positive PE sheath (arrows) which surrounds the nerve fasciculi (×275). FIG. 12 D. The transverse section of the denervated muscle spindle shows adenosine triphosphatase (ATPase) activity in the capsular perineural epithelium cells of muscle spindle (big arrows) as well as in the PE cell 11 covering (small arrows) of the extrafusal nerve fasciculus (E). Note the large sub perineural epithelial space created by the perineural epithelium cells 25, which encloses the muscle spindle completely (×300). These four histological transverse sections demonstrate the presence of sub perineural epithelial space in every nerve fasciculi including the muscle spindle, the potential space below the Perineural epithelium is connected to the SAS of the CNS, and play an important role in the transport of the therapeutic agents described here, administered at the ORE.

FIG. 18 is the Histological diagram 1800 drawn after extensive light and electron microscopic study of the myelinated nerve axons within the nerve fasciculi. It shows the longitudinal section of a myelinated axon (a) which bundles together to form the nerve fasciculi of the peripheral nerves. Diagram 14 a, b, and c shows the node of Ranvier 331 and the rest of the nerve fiber surrounded by the endoneurium 334, almost impermeable myelin sheath 330 with cytoplasm of Schwann cell 333, and axoplasm 332 that may transport (retrograde) minimum doses of insulin and other therapeutic agents to CNS. The insulin and therapeutic agents that enter the axoplasm have to enter the axon through the node of Ranvier 332 where the node does not have the myelin sheath to block the therapeutic agents' entry into the axons. The rest of the axon of the myelinated nerve fiber is not easily permeable to insulin and other therapeutic agents used in our invention to get into axoplasm, then transported to the CNS. That is why axoplasm plays a minor role in spread of insulin and other therapeutic agents to CNS, and most of the therapeutic agents transported through the sub perineural epithelium space and interstitial spaces within the nerve fasciculi. Our and other studies have shown that the potential subperineural epithelial space is the direct continuation of the SAS and CSF of the CNS and the perineural epithelium is the extension of pia arachnoid mater from the CNS to the peripheral nervous system (Shantha and Bourne IBID). This is specially so in the olfactory nerves, which are very short and the CSF from the olfactory bulb continuously permeating down from the SAS of the olfactory bulb along the sub Perineural epithelial, and nerve fascicular interstitial spaces all the way down to the olfactory mucosa. Insert b shows the details of the metabolically active Node of Ranvier lacking a myelin sheath; and allowing the absorption of insulin and therapeutic agents into the axoplasm from the interstitial space. Insert C is the section of the rest of the axon with a thick myelin sheath covering which is an obstacle for easy uptake of insulin and therapeutic agents into the axoplasm. Hence, once inside the nerve fasciculi, the insulin and the adjuvant therapeutic agents enter the axoplasm at the Node of Ranvier 331, to be transported retrograde to the CNS through neurotubules and axoplasm. The majority of the insulin and other therapeutic agents administered locally to treat Alzheimer's disease at ORE transported to the CNS through the sub Perineural epithelial and interstitial spaces within the nerve fasciculi to SAS and CSF of the CNS (Shantha T R and Bourne G H: The “Perineural Epithelium”: A new concept. It's role in the integrity of the peripheral nervous system. In Structure and Function of Nervous Tissues. Volume I. pp 379-458. (G H Bourne, Ed.). Academic Press, New York. 1969).

FIG. 19 is the diagram 1900 of the neruopil 367 between the ependymal linging of the central canal, ventricle 361 and the SAS 344 surrounding the brain (CNS) and the spinal cord. Note the epednyma lining 361 of the central canal and vetricles giving rise to tanacyes 362 which is branching and coming in contact with the neurons 364 and the rest of the neuropil which play a role in trasport of therapeutic agents from the CSF of the SAS and centrial canl including vetricles described in this invetion. The diagram also shows the microglia 362 in the neuropile and astroglial 363 end feet surrounding the BV 365 along with the pericytes and amorphous non cellular complex surrounding the BV to form the solid BBB. It also sends end feet to attach to the undersurface of the pia mater, and to come in contact with the ependymal lining and tanacytes 361,362. The end feet of the astroglia also surround the neuronal cell body and their processes 363. The ologodendroglia 366 send multiple extensions to surround the axons and form myelin sheaths in the central nervous system akin to the Scwann cells in the peripheral nerves. Note the thin, one or two layer thick pia mater 343 which is lined by astroglial end feet 363 towards the neuropil. Pia mater is carried into the cortex of the brain along with the penetrating BV 342 from the SAS of the spinal cord and the CNS to form the Virchow-Robin space 347 (see FIG. 16—#347). The CNS is surrounded by CSF in the SAS formed by the pia 343 and arachnoid mater 341 which is in turn surrounded by thick almost impermeable dura mater 340 firmly attached to the inside of cranial bones. The dura mater contains the large venous sinsus draining the CNS blood out of the brain to the jugular system. The neuropil 367 is shown with various neurons with nerve processes, the blood vessels 365 endowed with BBB, microglia 363, astroglia 363, oligodendroglia 366 and extensions of ependymal cells as tanacytes 362. The CSF in the central canal and ventricles 360 and in the SAS 344 with insulin and other therapeutic agents of our invention permeate the neuropile through the Virchow-Robins space (see FIG. 13—347), tanacytes 362, ependymal cells 361, CVO (FIG. 15), blood vessels with participation of CVVS, circumventricular organs and BV 365 carrying the pharmaceutical, biochemical, nutriceuticals, and biological therapeutic agents or compounds to the neuropile 367 to treat Alzheimer's disease as described in our invention. This diagram illustrates how the therapeutic agents of our invention reach their destination from the ORE to exert their therapuetic effect to cure and/or curtail signs and sympotoms of Alzheimer's disease (diagram modified from Grays Anatomy).

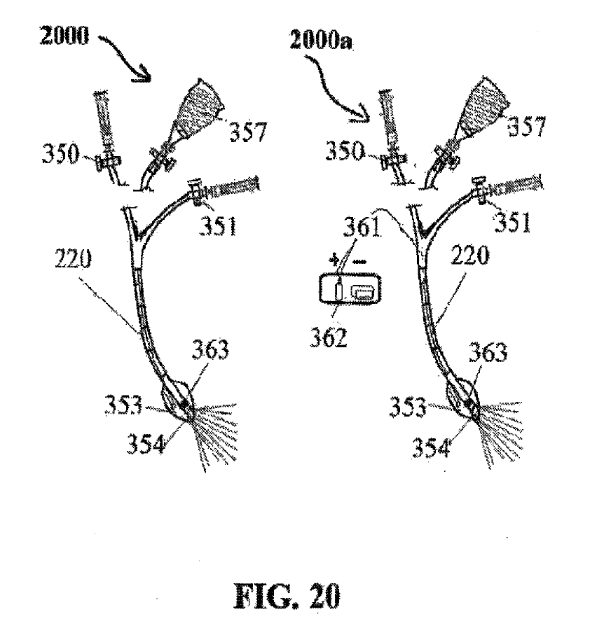

FIG. 20 is the diagrammatic presentation 2000 and 2000a of the special olfactory mucosal delivery inventive device 220 used to dispense therapeutic agents at olfactory mucosa and olfactory nerve (ORE) instead of respiratory mucosa (See FIG. 1a). This simple inventive device is included in the home delivery kit and designed for use by the medical staff, patients and caregiver at home, or clinics, or nursing homes, treating Alzheimer's disease, to prevent the delivery of therapeutic agents to the respiratory mucosa. The delivery device is made of a nontoxic semi rigid-flexible catheter made up of synthetic or semi synthetic material with 2 outlets 351, and 350. The outlet 350 used to attach any commercially available delivery sprayer nozzles 357, and syringe containing therapeutic agents of this invention, delivered to the ORE at the tip 354 at the anterior part of the ORE. The tip of the ORE delivery end catheter has a balloon 353, inflated with air or liquids to the desirable size through a syringe 351 to pass the tip through the nose, all the way to the anterior part of the roof of the nose without damaging (penetrating injury) the olfactory mucosa and nasal mucosa. The inflated balloon enclosing the tip prevents trauma caused by the tip of the catheter as the device introduced to the olfactory mucosal region. Further, the balloon 353 also holds the catheter in position without much movement when inflated. An LED bulb or fiber optic illuminator or other forms of tip sensor 363 used to locate the tip position of the delivery catheter in the nose and illuminate the tip. The tip or the distal end of the delivery catheter has a therapeutic agents' delivery opening 354. The LED illuminator or fiber optic tip 363 is conned to the battery power pack 361, 362, operated by AA, DC batteries or direct current with an ON and OFF switch connected to the tip of the catheter device by positive and negative wires 361 providing the electrical power source. As the catheter is passed through the anterior aspect of the nose bridge, the illumination is turned on which will show the location of the tip of the catheter through the skin of the nose to be properly placed for delivery of therapeutic agents to the ORE. The catheter tip advances slowly past the nasal bone and cartilage junction to deliver the therapeutic agents of our invention to the ideal location on the ORE. The 220 catheters open to the main part of the catheter 350 though which the therapeutic agents delivered to ORE. This device designed to have another delivery canula to deliver two or more separate therapeutic agents without mixing them (not shown in the diagram). A thin fiber optic nasal scope used to visualize the tip, a guide wire used through 350 canula to negotiate, and for the proper placement of the delivery catheter. The delivery catheter also has markings in millimeters and/or inches on the surface of the entire length of the catheter to indicate the how far the length of the catheter is inside the nose. Once the caregiver or the patient knows how far to negotiate (insert) the device, the next insertion will be easier. Diagram 2000 shows the device without the battery and LED bulb, and the diagram 2000a has electrical source 362 with LED bulb, connected by thin wires.

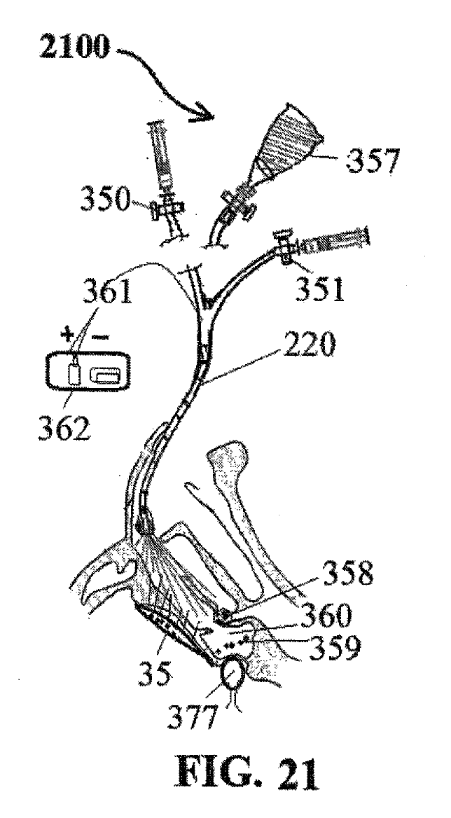

FIG. 21 is the diagrammatic presentation 2100 of the delivery catheter 220 used to deliver therapeutic agents of our invention described herein, on the olfactory mucosa and olfactory nerve (ORE) instead of the respiratory mucosa as described in the diagram 2000, and 2000a. This device placed in the nose to deliver therapeutic agents to treat Alzheimer's disease to ORE, olfactory bulb 35, sphenoid sinus 360, pituitary gland 362, sphenopalatine ganglion 358, and ten cranial nerves 359 in the cavernous sinus walls (five on each side). The therapeutic agents delivered to ORE are transported to the AD affected neuron embedded neuropile through the CVVS and circumventricular organs also besides the neural routes described herein. Note the tip of the catheter is at the anterior end of the ORE.

After lubricating the nasal passage, catheter tip; introduce the catheter past the vestibule of the nose, inflate the balloon, and advance the catheter directed upwards in the direction of inner canthus of the eye. As the balloon is inflated, apply pressure at the end of the nasal bone to locate the inflated balloon, and then pass another 0.75 inches to reach the appropriated anatomical therapeutic agents' delivery site on the ORE. The catheter passed with the patient in supine position; head extended with a neck support (see FIG. 1a). The therapeutic agents from this area pass on to olfactory bulb 35, sphenoid sinus 360, sphenopalatine ganglion 358, and five cranial nerves on the wall of the cavernous sinus 359, pituitary gland 362. The patients or caregivers trained to use this simple inventive special ORE delivery nasal catheter at home as described. Lubricants applied to the vestibule of the nose or catheter tip to facilitate the easy sliding of this device to the ORE.

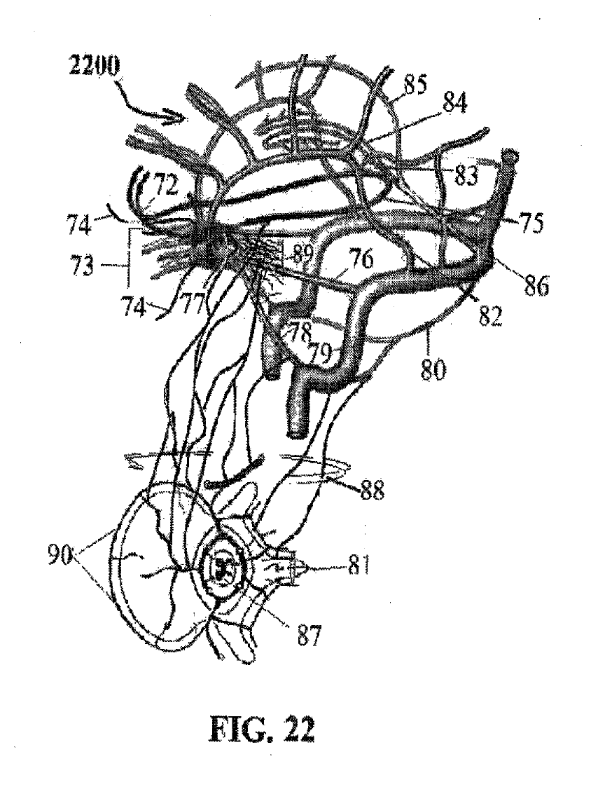

FIG. 22 is the diagram of the veins of the base of the cranium, ORE, brain, and vertebral anastomic veins 2200 showing the communication that forms the cranial vertebral venous plexus (CVVS) involved in the transport of therapeutic agents to the brain for the treatment of Alzheimer's disease and other neurodegenerative diseases.

Note how the veins from the olfactory mucosal region (ORE) 73, olfactory nerves, sphenoid sinus, spheno-ethmoid recess, superior meatus, sinuses of the nose specially ethmoid and sphenoid sinuses, pterygoid plexus of veins, and cribriform plate of the ethmoid bone 73 penetrate the basal part of the cranium and join the cavernous sinus 77 and other brain veins. The cavernous sinus also receives the venous blood from the ophthalmic veins 74. It receives tributaries from: Superior and inferior ophthalmic veins, sphenoid sinus, sphenoparietal sinus, and superficial middle cerebral veins. It also receives the veins of the superior and inferior petrosal sinuses as well via the emissary veins through the foramens of the skull (mostly through foramen ovale). There are also connections with the pterygoid plexus of veins via the inferior ophthalmic vein, the deep facial vein and emissary veins.

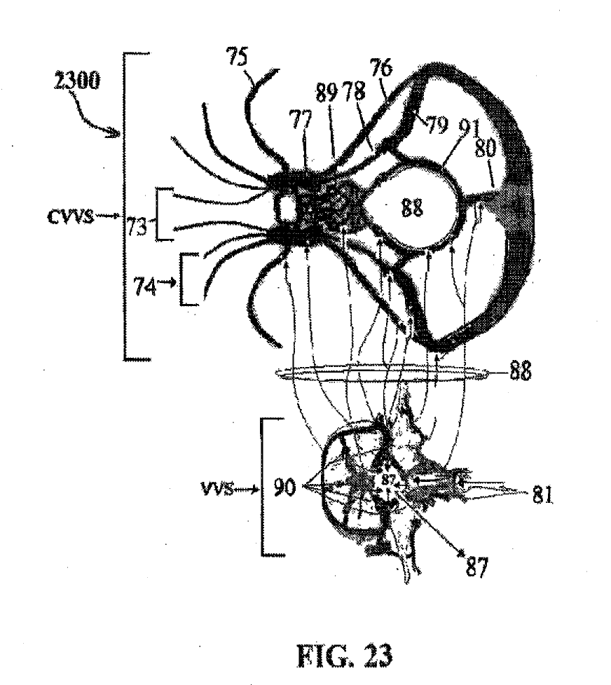

The cavernous sinus 77 has extensive communications with Veins from basal sinus and basilar plexus of veins 89. The cavernous sinus forms the center of the cranial vertebral venous plexus, which receives and drains to the rest of the venous system and leaks therapeutic agents in the blood into CSF of the SAS located close to its wall separated by very thin wall without any boney structures. The basilar plexus of veins 89 situated behind the pituitary gland on the dorsum sellae of the sphenoid bone. They continue with the veins of the clivus of occipital bone, on which the basilar plexus of veins are situated communicating with the veins around the foramen magnum 88 (see FIGS. 22, 23, #89, and 91) which in turn communicates with the cephalic end of VVS from the upper cervical region. They in turn communicate with the vertebral venous system of Batson 90 through foramen magnum 88, Anterior, superficial middle, deep middle cerebral veins 72, Basal Vein 75, and Occipital sinus 80. Cranial vertebral venous plexus (CVVS) also communicates with: Superior petrosal sinus 76, Inferior petorsal sinus 78, sigmoid sinus, transverse sinus 79, Inferior anstomic veins 82, Greater cerebral vein 83, Internal cerebral vein 84, Inferior sagittal sinus 85, Straight sinus 86, and Transverse sinus. These tributaries ultimately communicate, directly or indirectly, with cranial valveless vertebral venous system (CVVS) and the cavernous sinus which acts as a pooling and distributing center of therapeutic agents in the venous blood delivered from the ORE.

The diagram shows the possible valveless vertebral venous system 90 of the cervical vertebral region passing through the foramen magnum 88, directly connected to reach the basal venous plexus 89 and other veins of the brain to deliver insulin, bexarotene, ketamine, monoclonal antibodies, IGF-1, and cholinesterase inhibitor therapeutic agents to neuropil. Note that the therapeutic agents injected in to the cervical epispinal, interspinal, perispinal and epidural spaces 81 permeate to the CSF of the spinal cord (arrows) through the subarachnoid space to the CSF 87 and are transported to the rest of brain by CSF circulation. Once inside the CSF of the spinal cord, the denticulate ligament of the spinal cord directs the therapeutic agents through the CSF circulation to the front part of the brain stem and cerebral-pontine cistern and cisterna magna, from where they are transported to the rest of the basal part of the brain, brain stem, and cerebellum.

Though there is extensive communication between the cranial vertebral venous system and vertebral venous system, it is important to note that the cervical epispinal, perispinal and interspinal route 81 described by other investigators does not spread much of the therapeutic agents through the VVS to the brain. It is the permeation and passage of the therapeutic agents from these anatomical sites (arrows in vertebral body) to the SAS through the arachnoid villi (Shantha T R and Evans J A: Arachnoid Villi in the Spinal Cord, and Their Relationship to Epidural Anesthesia. Anesthesiology 37:543-557, 1972. Shantha T R and Bourne G H: Arachnoid villi in the optic nerve of man and monkey. Expt Eye Res 3:31-35 (1964)) associated with vertebral venous system, Virchow-Robin space of nerve roots, nerve root sub Perineural epithelial, and nerve fascicular interstitial spaces (Shantha IBID), subdural space, inter-arachnoid spaces which are responsible for the spread of therapeutic agents from the epispinal, perispinal, interspinal, and other vertebral venous plexus, and epidural space to CSF. The direct spread of therapeutic agents as described U.S. Pat. No. 8,119,127 B2 from the cervical epi, peri and interspinal space and epidural space veins to the brain venous system by cervical vertebral venous system of Batson is minimal to exert therapeutic effect in the neurons and neuropil of the CNS. This is due to gravity and the bidirectional flow of blood that feeds venous sinuses to the CNS blood vessels (CVVS) as shown in the diagram (FIGS. 22, 23). It is the transport of these therapeutic agents to SAS and CSF (arrows 87), which plays a role in the spread of these therapeutic agents to the brain and spinal cord for the treatment of Alzheimer's disease and other neurodegenerative diseases.

The spread of prostate cancer to the vertebral venous system of Batson (VVS) cannot be compared or extrapolated to the similar spread of therapeutic agents to brain, from the epi, inter, and perispinal injection site, spread from the cervical vertebral venous system to the brain. The physical forces involved in the spread in lumbar-sacral VVS and Cervical VVS are different. In VVS, there is constant raise and fall of pressure in valveless pelvic plexus of veins. This is due to bowel movement, staining, coughing, bearing during defecation and urination, weight lifting, eating, bending, and other physical pressures create a pushing pulsatile force on prostate cancer cells emboli-mets located in these valve less veins, which are literally pushed from the prostate to retrograde spread to valveless VVS to vertebral bodies. Such a physical force component does not consistently continue in the epi, inter, perispinal space of the cervical region and there is negative pressure the connecting tributaries of these veins due to gravitational pull. Further, the therapeutic agents are liquid to stay in the VVS vein for a long time as particulate matter to be pushed up against gravity in the cephalad direction due to physical force as it happens in the spread of the prostate metastatic cancer cells in the Batons' plexus of veins. The liquid therapeutic agents dissipate rapidly to the surrounding tissue spaces. Similarly, the lung abscess (Empyema-lung infection) spreads to the brain due to constant change in the thoracic VVS positive pressure due to coughing and respiratory movements with infected emboli dislodged, pushed to the VVS, then to the CVVS resulting in brain abscess. If it were that easy to spread as described, then every case of pneumonia, bronchitis, lung tumors, and pleural pathology would have landed in the brain in most of the cases, which is not the case.

Further, the gravity in upright position creates a negative pressure in cervical VVS (see FIGS. 22, 23), resulting in the prevention of venous flow upwards, hence the cranial spread of therapeutic agents from cervical epi, inter, perispinal administration to the brain in any considerable quantity to be therapeutically effective. By the time a favorable situation created for cephalad spread, the therapeutic agents have dissipated in the surrounding tissue, lymphatics, and the veins, unlike prostate cancer and lung abscesses emboli. For these reasons, the most important spread of therapeutic agents deposited at cervical and other epi, inter, perispinal and epidural route of the vertebral column is; through the routes enumerated above. That is through the dorsal and ventral nerve root arachnoid villi, and their association with veins with the VVS (see FIGS. 22, 23 arrows, 24, 15, and 26), epidural and subdural spaces, inter arachnoid spaces, thinned out dura at the entrance of the dorsal and ventral roots, and sub Perineural epithelial, and nerve fascicular interstitial spaces of nerve roots, to CSF in the SAS 87. From here, it spreads by cephalic route to the CNS through CSF circulation as described above. Direct spread of epi, inter, perispinal route through the cervical VVS sinus connection through the foramen magnum (see FIG. 22, 23) is only speculative at best and therapeutically minimal to cure or curtail the neurodegenerative diseases. So far, there is no histological and experimental radioisotopes study tracing such a route of transport directly to the CNS through VVS from the epi, inter, and perispinal cervical venous routes. Hence, it is the spread from the sites described herein to SAS CSF 87, which is responsible for such a transport of therapeutic agents of our invention in curing and curtailing neurodegenerative diseases including Alzheimer's disease. The spread of therapeutic agents to cisterna Magna may play an important role in treating neurodegenerative diseases if the insulin, bexarotene, ketamine, monoclonal antibodies, IGF-1, and cholinesterase inhibitor therapeutic agents deposited, close to or delivered to the cistern through cisterna magna puncture like lumbar puncture or to the SAS delivery of therapeutic agents at the cervical region.

Once the therapeutic agents are transported from the CVVS to cavernous sinus 77; the CSF in the SAS, they come in contact with numerous structures from the delicate wall of this venous sinus. Examples are: the oculomotor nerve (CN III), the trochlear nerve (CN IV), the ophthalmic nerve (the V₁ branch of the trigeminal nerve), the abducens nerve (CN VI), and the internal carotid artery with sympathetic and parasympathetic plexus. The optic nerve lies just above and outside the cavernous sinus, superior and lateral to the pituitary gland on each side, and enters the orbital apex via the optic canal. These structures bathed in the therapeutic agents delivered through the CSF, through the CVVS, VVS and cavernous sinus that transport them to the Alzheimer's disease afflicted CNS.

FIG. 23 is the diagram of the veins of the base of the brain and vertebral anastomic veins 2300 showing the communication that forms the cranial vertebral venous system (CVVS) involved in the transport of therapeutic agents for the treatment of Alzheimer's disease and other neurodegenerative diseases.

This diagram describes the cranial valveless vertebral venous plexus system and its connection to the cervical vertebral venous system (plexus) of Batson (VVS), and the role they play in the transport of therapeutic agents. Note how the veins from the olfactory mucosal region 73, olfactory nerves, sphenoid sinus, sphenoid-ethmoid recess, superior meatus, pterygoid plexus of veins 73, penetrate the cribriform plate of the ethmoid bone and how the basal plexus of veins 89 from the cranium join the cavernous sinus 77. The cavernous sinus also receives the venous blood from the ophthalmic veins 74. The cavernous sinus 77 has extensive communications with the Veins form basilar plexus of veins 89, which communicates with the cervical vertebral venous system of Batson 90. There is a venous circle 91 around the foramen magnum 88, which communicates with the tributaries from sigmoid sinus 79, inferior and superior petrosal veins 76, 78, and in front with the basilar plexus of veins 89, which drain into and from cavernous sinus veins 77. Through this extensive network of veins, the sphenoid sinus 75 also joins the cavernous sinus 77.

The cervical vertebral venous system 90 (VVS), passes through the Foramen magnum 88 (shown as multiple arrow markings), reach the venous circle 91 around the foramen magnum 88. From here it reaches the basilar venous plexus 89 and veins around the foramen magnum 91 located on the dorsum sellae and the clivus of the sphenoid bone, which continues with the veins on the clivus of the occipital bone. The cranial vertebral venous plexus or system (CVVS) communicates with the veins around the foramen magnum and with the cervical part of the vertebral venous system, and the flow of blood is bidirectional. The diagram 90 shows these veins of VVS connected to the cranial vertebral venous plexus through the venous channels (arrows) around the foramen magnum 88 and directly with other vein tributaries of the cavernous sinus 77. Both the right and left cavernous sinuses communicate freely with each other by the anterior and posterior communication sinus around the superior surface of the pituitary gland. The therapeutic agents in the blood of the cavernous sinus freely diffuse through the delicate wall with CSF of the adjacent SAS. Through this media, the therapeutic agents from the ORE are transported to CSF and then to the brain. The VVS communicates with the SAS of the spinal cord 87 (arrows), which facilitates the transfer of therapeutic agents cephalad to the CNS injected at epi, inter, perispinal 81 and epidural sites. Due to gravity and other physical forces, hardly any therapeutic agents reach in appreciable amount to exert therapeutic effect through the direct bidirectional venous communication between the CVVS and VVS. Their main role is to carry the therapeutic agents and deliver them to CSF in the SAS 87 to exert a curing or curtailing effect on the afflicted CNS neuronal complex.

Note that the therapeutic agents injected in to the epispinal, interspinal, perispinal and epidural spaces permeate to the CSF (arrows 87) of the spinal cord through the subarachnoid space to the CSF 87 and transported to the rest of brain. Once inside the CSF of the spinal cord, the denticulate ligament of the spinal cord directs the therapeutic agents through the CSF circulation to the front part of the brain stem and cerebro-pontine cistern as well as cisterna magna, and then distributes them to the rest of the basal part of the brain and brain stem and cerebellum. The spread of prostate cancer (particulate matter, not liquid) to the vertebral venous system of Batson is entirely due to physical mechanism involving micro emboli, which is different from the spread of therapeutic agents injected epi, inter, perispinally in liquid form as described herein.

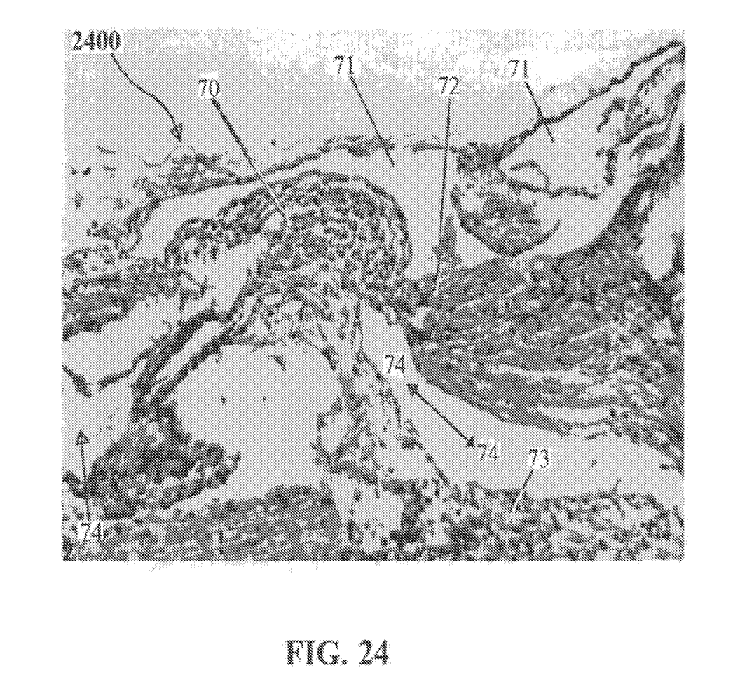

FIG. 24 is Longitudinal section 2400 through spinal nerve roots from the monkey, showing a typical arachnoid villi 70 protruding into epidural veins 71 outside the dura. Note that the dura 72 is breached by arachnoid thus creating a week spot on the membrane for transfer of perispinal and epidurally injected therapeutic agents to SAS CSF. The villi probably formed by the multiplication of the Perineural epithelium surrounding the nerve root 73 continuous with the arachnoid and pia mater of CNS. The sub Perineural epithelial, and nerve fascicular interstitial spaces 74 become continuous with the SAS. The villi have intercellular pores, which leak the fluids back and forth from the vein to CSF of the SAS to tissue spaces, VVS veins surrounding the epidural and perispinal space, and vice versa. The epi, peri, interspinal and epidurally introduced therapeutic agents find their way through the villi and other tissue spaces connected to the VVS and nerve roots into the CSF to be distributed to the CNS. The veins of epidural space are in close proximity to the SAS; thus leak the therapeutic agents to the CSF in the SAS. X264, reduced from X280

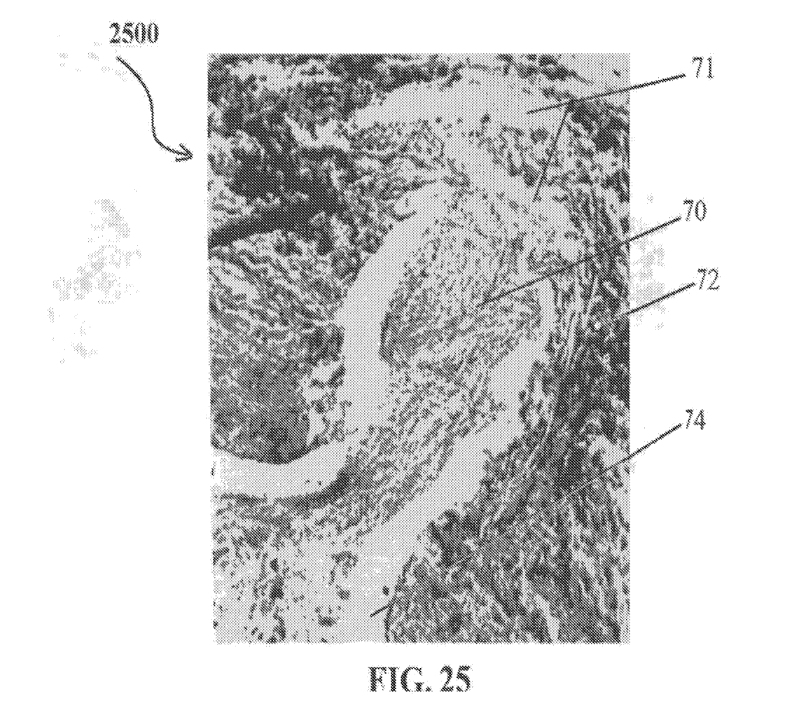

FIG. 25 is a section through the human spinal nerve root 2500, showing the arachnoid proliferation in the form of a villus 70 penetrating the dura 72 in close proximity to the epidural and perispinal veins 71. Note that the dura 72 completely breached by this protrusion of arachnoid villi. The villi formed by the Perineural epithelium surrounding the nerve root that become continuous with the arachnoid and pia mater of CNS. The sub Perineural epithelial, and nerve fascicular interstitial spaces 74 become continuous with the SAS. The villi have intercellular pores, which leak the fluids back and forth from the vein to CSF of the SAS to tissue spaces, VVS veins surrounding the epidural and perispinal space, and vice versa. The epi, peri, interspinal and epidurally introduced therapeutic agents find their way through the villi and other tissue spaces connected to the VVS and nerve roots into the CSF to be distributed to the CSN. The veins of epidural space are in close proximity to the SAS to leak the therapeutic agents to the CSF in the SAS. ×65, reduced from ×74 magnification.

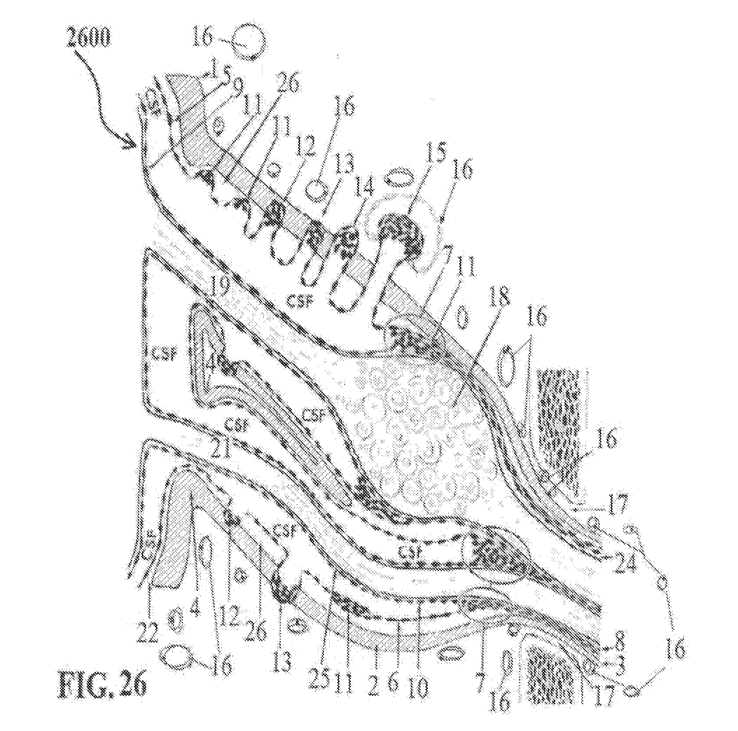

FIG. 26 is the drawing 2600 of the histology of the spinal cord, dorsal and ventral roots, dorsal-root ganglion, and common nerve trunk as they emerge from the spinal and cranial nerve foramina based on extensive histological studies (shantha and Evans IBID). The numbering from the original publication is unchanged, so that it represents the original concept of the research studies. These diagrams show the relationship of spinal and root meninges to membranes of the peripheral nerve. Note the continuation of spinal epidural, subdural, and subarachnoid spaces with dorsal and ventral spinal roots for some distance. The pia arachnoid membrane 6, 10 of the spinal roots continues as perineural epithelium of peripheral nerves 8, 24 as they emerge out of inter-vertebral foramen 17. As Perineural epithelium 8, 24, extends to the CNS on the nerve roots in close proximity to the spinal cord, frontal part of the base of the brain and brain stem, it separates to form distinct pia and arachnoid mater 5, 6, 9, 10, and 25 of emerging nerve roots and CSN. Epi and perineural connective tissue 3 around the nerve roots become continuous with the dura mater 1, 2, 4. There are arachnoid proliferations 7, 11 also as the pia and arachnoid join to form the Perineural epithelium of the nerve roots as they emerge from the vertebral canal 17 and brain stem. Marked and unmarked circles 16 indicate the epi, peri, inter, and epidural venous plexus. Note that the CSF from the SAS permeates all the way on the nerve roots of both cranial and spinal nerves and acts as a transporter of neurotrophic substances form the CNS and transmitter of therapeutic agents from the perispinal and epidural spaces and VVS and CVVS to the CNS. The CSF surrounds the dorsal root ganglion 18. The spinal-cord and spinal root subpial space and subperineural epithelial spaces are only potential spaces and are continuous with each other 23, 24, 25. There is a distinct potential subdual space 6, 26 also which can easily transport therapeutic agents from the perispinal and epidural space to the CSF of the SAS. The Inter perineural epithelial space continues with inter arachnoid spaces 6, 24. Various types of arachnoid villi given off from the nerve root arachnoid mater are also illustrated 11, 12, 13, 14, and 15. Note the relationship of the arachnoid villi 15 to epidural-perispinal vein 16 that transports therapeutic agents from the VVS, CVVS to arachnoid villi, then to pores in the arachnoid villi to SAS CSF. The multiple unnumbered round circles of different size represent epi, inter, peri-spinal and epidural valveless veins that transport the therapeutic agents from these anatomical areas to the SAS CSF of the spinal cord and the brain. The location of the Dural collar 4 as the nerve roots emerge from the spinal subarachnoid space is also shown. (FIGS. 24, 25, and 26 are reproduction from Shantha T R and Evans J A: Arachnoid Villi in the Spinal Cord, and Their Relationship to Epidural Anesthesia, Emory University School of Medicine. Anesthesiology 37:543-557, 1972).

Iontophoresis Application Using the Delivery Device Described Herein to Facilitate the Rapid Uptake and Distribution of Large Molecules of Therapeutic Agents to the CNS from the Olfactory Mucosal Region (ORE) Bypassing Bbb

Iontophoresis is a method for enhancing and facilitating the delivery of the therapeutic agents across the mucous membrane and skin. This method gets around the barrier imposed for the penetration and permeation of large molecular weight therapeutic agents from olfactory mucosa to deliver anti Alzheimer's disease therapeutic agents to the CNS. It uses electrical current to activate and to modulate the diffusion of a charged molecule across a biological membrane (in this case olfactory mucosa), such as the skin or mucous membranes, in a manner similar to passive diffusion under a concentration gradient, but at a facilitated rate. In general, iontophoresis technology uses an electrical potential or current across a semi permeable olfactory mucosal barrier. Delivery of therapeutic agents to patients has been shown using iontophoresis to facilitate the drug delivery by enhancing the permeability of the barrier membranes. The technique uses low direct current to drive charged species (therapeutic agents) into the olfactory mucosa, then to transport them to the olfactory bulb, trigeminal nerves, and sphenopalatine ganglion, cranial vertebral venous plexus, and deliver them to the brain involved in the Alzheimer's disease.

The technology based on the principle that an electric potential will cause ions in solution to migrate according to their electrical charges. The quantity and distribution of a drug delivered by iontophoresis is dependent upon the charge of the ion, the size of the ion (molecular weight), the strength of the electrical current being applied, electrode composition, the duration of current flow and numerous other factors such as pH, ionization, molecular weight of the therapeutic agents, uptake enhancers and so forth.

The method utilizes pulsed electric fields and has an advantage of allowing lower concentrations of compositions utilized as opposed to high dosages typically used with passive delivery modalities (oral and parenteral administration). The method of the lontophoresis and delivery of therapeutic agents of this invention are incorporated in the delivery catheter described in FIGS. 5 to 10. It provides a delivery system that allows controlled sustained, high local concentrations of therapeutic agents such as insulin, bexarotene, and ketamine, monoclonal antibodies, IGF-1, and cholinesterase inhibitor therapeutic delivered directly at a site of olfactory mucosa without exposing the entire circulation and rest of the body to the therapeutic agents.

The method includes administering the composition of insulin, bexarotene, ketamine, monoclonal antibodies, IGF-1, and cholinesterase inhibitor therapeutic agents to the subject and applying an electrical impulse to olfactory mucosa (ORE) via lontophoresis (FIGS. 5-10) wherein the impulse is of sufficient strength and time for the impulse to cause lontophoresis, thereby resulting in sustained delivery. This methodology utilized (turned on) after placing the device described here in on the olfactory mucosa and introducing the therapeutic agents as shown in diagrams 5-10. The term “sustained” as used herein means that once the composition is delivered to the ORE, it is retained in the ORE for a period of time of as long as 24, and typically for 12 hours. In other words, there is no appreciable washout of the composition as compared with the concentration of the composition delivered under conventional delivery (e.g., passive diffusion).

The therapeutic compositions administered alone or in combination with each other or with another agent to treat Alzheimer's disease. The first electrode (+) is preferably made of an electrically conductive material that is biologically compatible, e.g., biologically inert, with a subject. Examples of such material include silver or platinum wire wrapped around synthetic material. The second electrode (−) is placed a bit further distal to the therapeutic agents' delivery pores on the device 220 (FIG. 5-10 #520). The first and the second electrodes coupled to the voltage source (FIG. 5-10, #517). The conduction wires are connected between the microprocessor unit and the active (positive) and passive (negative) electrodes. The electrodes made up of, for example, silver, or platinum wires, but can be any conductive composite material. The voltage is about 60 V and the pulse parameters, for example, are four pulses delivered at 1 Hz each of 40 milliseconds. The current can be direct, alternate, or pulsed, and can have various waveforms, including square, sinusoidal, triangular, and trapezoidal. The more complex forms may not be of much advantage as direct current is most commonly used. Square wave pulses known to be gentler, hence such electrical wave pulses chosen and delivered to the delicate olfactory mucosa.

Iontophoresis enhances the uptake of therapeutic agents for the treatment of Alzheimer's disease through the olfactory mucosa and other passage routes (sphenoid sinus walls, nerve fasciculi, and cranial vertebral venous plexus) described by these following mechanisms:

a) Ion-electric field interaction provides an additional force that drives ions through the olfactory mucosa,
b) The flow of electric current increases the permeability of the olfactory mucosa, and
c) Electro-osmosis produces the bulk motion of the therapeutic agents that carries ions or neutral species with the solvent stream that are carried to the CNS by the explained routes described here.
d) The passage of therapeutic agents is between the olfactory nerve dendrites, at the tip of the new generation of cells sprouting, the pores left by the dying olfactory neurons and sustenticular cells, spaces between the sustanticular cells and receptors cells, openings of the Bowman's glad's ducts, and also the microvilli pinocytosis on the olfactory mucosal surface.

Electrical energy assists the movement of ions across the olfactory mucosa using the principle “like charges repel each other and opposite charges attract”. The operator or the patient selects a current from the electrical output manipulator (FIGS. 5-10, #517) below the level of the patient's pain threshold and allows it to flow for an appropriate length of time. Iontophoretic wires on the delivery catheter are located ideally at the site of the olfactory mucosa and sphenoid sinus with the control box outside the nose.

The Iontophoresis Delivery Unit application is contraindicated for use on patients with electrically sensitive support systems e.g., pacemakers, drug delivery devices, and patients with a known allergy or sensitivity to the drugs to be administered.

Anatomic Histology of the Olfactory Mucosa, Olfactory Nerves and Olfactory Bulb and its Connections to CNS Cortical Centers where this Inventive Devise is Positioned to Deliver Therapeutic Agents to Treat Alzheimer's and Other CNS Dieseases Using Insulin, Bexarotene, Ketamine, Monoclonal Antibodies, IGF-1, and Cholinesterase Inhibitor Therapeutic Agents Described Here.

The olfactory epithelium is a specialized ten million neuro epithelial cells inside the nasal cavity that are involved in perception of smell, located in the dorsoposterior aspect of the nasal vault (see FIGS. 1, 2, 3). Because the olfactory neuronal cells are the only surface neural cells in the body; olfactory mucosa are considered in this aspect as a “window to the brain” and an entryway too for many therapeutic agents to reach the CNS bypassing blood vessels of the BBB. Due to close proximity of olfactory mucosa, separated by thin perforated cribriform plate of the ethmoid bone, the therapeutic agents transported to the olfactory bulb and rest of brain rapidly.

Interestingly, the human adult olfactory mucosa is a potential source of olfactory ensheathing cells and multipotent neural stem cells. They have been used in autologous transplantation therapies aimed at the treatment of degenerative or traumatic conditions of the central nervous system, such as spinal cord injury or Parkinson's disease (Mackay-Sim A et al (2008) Autologous olfactory ensheathing cell transplantation in human paraplegia: a 3-year clinical trial. Brain 131(Pt 9):2376-2386. Murrell W et al (2005) Multipotent stem cells from adult olfactory mucosa. Dev Dyn 233(2):496-515).

It is demonstrated that the anatomical configuration of the nasal cavities affects the olfactory airflow and the fraction of the air stream entering the naris that reaches the olfactory cleft is only between 10 and 15% (Horning D E (2006) Nasal anatomy and the sense of smell. Adv Otorhinolaryngol 63:1-22. Hahn I, Scherer P W, Mozell M M (1993) Velocity profiles measured for airflow through a large-scale model of the human nasal cavity. J Appl Physiol 75(5):2273-2287). Hence, the delivery of therapeutic agents by using nasal sprays is ineffective. Most of the nasal sprays will not deliver the therapeutic agents to the ORE. That is why, to deliver all of the therapeutic agents to olfactory mucosa (ORE), a special delivery catheter designed as described herein in FIGS. 5-11. Ordinary nasal sprays result in depositing therapeutic agents in respiratory mucosa, hardly any on the olfactory mucosa. To deposit the therapeutic agents on the ORE, the patient has to be placed in a supine position with head extended (FIG. 1a) and use this special delivery device described here.

Humans have about 10 cm² (1.6 sq in) of olfactory epithelium, whereas some dogs have 170 cm² (26 sq in). A dog's olfactory epithelium heavily innervated, with a hundred times more receptors per square centimeter. Olfactory mucosa in humans lies on the roof, the upper lateral, and medial walls of the nasal cavity five to seven centimeters above, and behind the nostrils. The human olfactory mucosa consists of a pseudo-stratified columnar epithelium resting on a vascular and cellular lamina Propria. Histological study show that the olfactory epithelium consists of 4 distinct cell types:

I. Olfactory cells of the epithelium (ten million) are bipolar neurons, which congregate to form the olfactory nerve (cranial nerve 1). They are responsible for conducting the electrical impulses to the olfactory bulb and rest of the CNS. As they emerge to the lamina propria, they form up to ±20 olfactory nerve fasciculi surrounded by Perineural epithelium and sub Perineural epithelial space, which conduct the therapeutic agents to the SAS and CSF surrounding the olfactory bulb and olfactory tracts. From there, the therapeutic agents transported to the rest of the CNS (Shantha T. R. and Yasuo Nakajima. Histological and Histochemical Studies on the Rhesus Monkey (Macaca Mulatta) Olfactory Mucosa. Yerkes Regional Primate Research Center, Emory University, Atlanta, Ga.: Z. Zellforsch. 103, 291-319 (1970). Some of the therapeutic agent's fron ORE absorbed by the olfactory neurons dendrites are conducted through the axons to olfactory bulb. However, the therapeutic agents have to bypass many neuronal synapses such as olfactory glomeruli (1 glomeruli for 5555 olfactory axons, a total of 1800 glomeruli). They pose a daunting obstacle for the rapid conduction of therapeutic agents to the CNS through olfactory nerve axons as perceived mistakenly by many investigators. The therapeutic agents have to pass through the synapses of mitral and periglomerular and tufted cells. Thus the spread of the majority of the therapeutic agents from the olfactory mucosa, and olfactory nerve to the CNS takes place trough the sub Perineural epithelial space, inter-fascicular spaces, between the endoneurium of the olfactory nerve, sphenopalatine ganglion nerves and trigeminal nerves (Shantha T R and Bourne G H: The “Perineural Epithelium”: A new concept. Its role in the integrity of the peripheral nervous system. In Structure and Function of Nervous Tissues. Volume I. pp 379-458. (GH Bourne, Ed.). Academic Press, New York. 1969).

II. Supporting cells: Analogous to neural glial cells are the supporting cells (sustentacular cells) of the olfactory epithelium.

III. Microvillar cells: These cells first described in 1982 are the second type of morphologically distinct class of chemoreceptor in the human olfactory mucosa. However, their putative role in the olfaction has not definitely demonstrated.

IV. Basal cells divided into two types.

a. The horizontal basal cells line the olfactory epithelium and the slightly more superficial globose basal cells thought to be the primary stem cell.

b. Brush Cells resting on the basal lamina of the olfactory epithelium are stem cells capable of division and differentiation into either supporting or olfactory cells. The constant divisions of the basal cells lead to the olfactory epithelium replaced every 2-4 weeks.

Bowman's (olfactory) Glands deliver a protenacious secretion via ducts onto the surface of the mucosa. The role of the secretions is to trap and dissolve odiferous as well as therapeutic agents to transport to the bipolar neuronal pathways, Perineural epithelium, sub Perineural epithelial space to the olfactory bulb, SAS and CSF. During the Iontophoresis procedure, the opening of the glands on the surface can play an important role in delivery of large MW therapeutic agents to the sub Perineural epithelial space and blood vessels, then to olfactory bulb and CNS. The therapeutic agents deposited in the opening of these glands can stay for long periods and exert therapeutic action slowly.

Delivery of therapeutic agents to the olfactory nerves in the olfactory mucosa (see FIGS. 1-4) results in transport of insulin, bexarotene, ketamine, monoclonal antibodies, IGF-1, and cholinesterase inhibitor therapeutic agents' transport to the olfactory nerve fasciculi, olfactory bulb, and olfactory tract to various nuclei in the CNS as shown in FIG. 14. As a neural circuit, the olfactory bulb has one source of sensory input (axons from olfactory receptor neurons of the olfactory epithelium), and one output (mitral cell axons). As a result, it is assumed that it functions as a filter, as opposed to an associative circuit that has many inputs and many outputs. However, the olfactory bulb also receives “top-down” information from such brain areas as the amygdala, neocortex, hippocampus, locus coeruleus, and substantia nigra. Due to these complex histological obstacles posed by the histological structure, the therapeutic agents transport in the axons and dendrites is minimal at best.

The combination of olfactory mucosa lontophoresis with electrical stimulation and delivery of therapeutic agents through the olfactory mucosa is the most important method of treatment for Alzheimer's, senile dementia and other CNS diseases described above. The anterior ethmoidal nerve situated in front of the olfactory mucosa and sphenopalatine ganglion behind the olfactory mucosa (FIG. 1-6, #107), also carries the therapeutic agents to the brain stem nuclei through the ophthalmic and maxillary branches of the trigeminal nerve, and cranial vertebral venous plexus.

Hundreds of studies have shown that the olfactory mucosa with olfactory nerve transports many therapeutic agents directly to the brain by passing the BBB (see references cited). Hence, it is a useful anatomical site for the delivery therapeutic agents with producing lontophoresis and electroporation. The device described herein incorporates lontophoresis embodiments applied to make the olfactory mucosa open up (leak) to deliver large molecules size therapeutic agents to the CNS by bypassing arterial system of the BBB (FIGS. 5-10).

Anatomy of the Sphenopalatine Ganglion and its Connection to the CNS Cortical and Brain Stem Centers for the Treatment of Alzheimer Disease Using Therapeutic Agents with the Inventive Device to Deliver Therapeutic Agents (FIGS. 1-4, 6,7)

The sphenopalatine ganglion (synonym: Meckler's ganglion, ganglion pterygopalatinum, nasal ganglion, pterygopalatine ganglion) is the largest parasympathetic ganglion in the body found in the pterygopalatine fossa associated with the branches of the maxillary nerve (FIG. 2). It is ideally located close to the olfactory mucosa on the lateral wall of the nasal cavity immediately below the sphenoid sinus. The sphenopalatine ganglion supplies the lacrimal gland, paranasal sinuses, glands of the mucosa of the nasal cavity and pharynx, the gums, and the mucous membrane and glands of the hard palate and cerebral blood vessels, which form Circle of Willis and its branches. It has extensive nerve connections to and from CNS to ganglion, which transmit therapeutic agents to the brain and brain stem through the sub Perineural epithelial, and nerve fascicular interstitial spaces (see FIGS. 16, 17, 18) as described above when therapeutic agents are deposited in the olfactory mucosal region (ORE). When we say the stimulation or delivery of therapeutic agents of sphenopalatine ganglion, it includes any and all of these communicating branches of the ganglion described here. The Sphenopalatine ganglion receives a sensory, a motor, parasympathetic, and sympathetic roots. The parasympathetic nerves on the BV play an important role in dilatation of these cerebral blood vessels and make BBB more permeable to therapeutic agents.

Circumventricular Organs and Therapeutic Agents Transport to Neuropile from CSF in the Treatment of Alzheimer's Disease

Therapeutic agents for treating Alzheimer's disease get into the CSF and then into the neuropile through these circumventricular organs, as described in FIG. 15. The therapeutic agents are also transported to the neuropile through the Ependymal lining, pia mater, Virchow Robin space, arachnoid villi, nerve roots, cranial vertebral venous plexus, nerve root lymphatics (which play a minimal or no role), and the choroid plexus which plays a role in the transport of therapeutic agents. The spread of the therapeutic, pharmaceutical, biochemical and biological agents or compounds into the neuropile through these circumventricular organs to treat Alzheimer's disease enhanced by the use of this invention insulin, with bexarotene, ketamine, monoclonal antibodies, IGF-1, and cholinesterase inhibitor therapeutic agents as part of the therapy. Once the therapeutic agents enter the CSF in the SAS, they have free access to the neuropile by passing the arterial BBB through these circumventricular organs (FIG. 15). These are additional routes by which the therapeutic agents delivered to the brain in the treatment of Alzheimer's, and other neurodegenerative diseases.

It is important to note that the BV, especially the communicating venous system of the olfactory area of the nose, ethmoidal sinuses, sphenoid sinus, and the eyeball are in direct contact with the cavernous venous sinus plexus (FIGS. 22, 23). This in turn is in contact with the brain venous system around the pituitary gland, and nerve roots, which communicate with the CNS at the neurovascular interface of the hypothalamus-hypophysis system and with complex venous sinuses within the cranium. They form the cranial end of the Batson's vertebral venous system (CVVS) without any valves (FIGS. 22, 23). From these sites, the therapeutic agents spread from the olfactory nasal area (ONA-ORE), sphenoid sinus, sphenoethmoidal recesses, diploic veins, upper nasal sinuses such as ethmoidal sinuses, upper posterior wall of the nasal cavity, and the eyes to the CNS through various weak BBB systems of circumventricular organs, and the cranial vertebral venous system (CVVS) venous network.

The Cranial-Vertebral Venous System (CVVS) of the Olfactory Area of the Nose and its Connection to the CNS by Passing BBB to Deliver Therapeutic Agents of this Invention to Treat Alzheimer's Disease

The cranial-vertebral venous system (CVVS) is one of the routes of transport of therapeutic agents from the olfactory mucosal and olfactory nerve area (FIGS. 1, 1a, 2, 3, 22, 23) besides the nerve route described above. The CVVS includes the communicating venous system from:

I. the olfactory mucosa; olfactory nerves, lamina propria,

II. anterior part of roof of the nose; and

III. posterior aspect of olfactory mucosa which includes under surface of the sphenoid sinus, sphenopalatine ganglion and

IV. Sphenoid-ethmoidal recess, superior nasal meatus, superior turbinate,

V. cribrifrom plate of the ethmoid bone, and ethmoid sinuses,

VI. anterior surface of the first, second and third cervical vertebrae and venous sinuses in the epidural space,

VII. sphenoid and ethmoid sinuses with its cavernous sinus on the walls, sell turcica, the cranial nerves in the wall of the sphenoid sinus and ORE, and

VIII. the ophthalmic veins of the eye ball entering and leaving the cranium through the superior and inferior orbital fissures connecting to the cavernous sinus, superior and inferior petrosal veins, basal veins; the interconnecting veins on the roof of the sella turcica, veins of the infundibulum of the pituitary gland, diploic veins of the base of the cranial bones, veins of the middle, and internal ear and their connection to petrosal veins on the petrous part of the temporal bones; anterior, superficial middle, and deep middle cerebral veins; vein of Galen (great cerebral vein-formed by the thalamostriate veins and choroid veins) and,

IX. Diploic veins which are connected with the Dural venous sinuses, supraorbital veins, and to the sphenoparietal sinus, deep temporal veins, through an aperture in the great wing of the sphenoid bone and into the confluence of the sinuses.

X. Blood vessels of the circumventricular organs communicating with the above described veins and intra cerebral veins,

XI. Vertebral venous system of the upper cervical vertebrae, that connects the cranial end (described above—FIGS. 22, 23), and caudal vertebral venous system of Batson, through the foramen magnum extending up and down from these sites.

The veins from these regions form the cranial end (CVVS) of the caudal vertebral venous system (VVS). The cranial vertebral venous plexus we describe here is more extensive than the VVS of Batson in the pelvis and lower sacral and lumbar vertebrae. The CVVS and VVS are an interconnected plexus of valve less veins that surround the spinal cord vertebrae and extend the entire length of the spine from the cranium communicating with the venous system of the brain, all the way down to the pelvis and connected to the pelvic plexus of veins (Prostatic plexus of veins in the male). This extensive valveless venous system is available for a vascular route of transfer of therapeutic agents to the CNS inside the cranium, which richly involves the vertebrae and basal part (floor) of the cranium, which we call Cephalad Cranial Vertebral Venous System (CVVS).

Valves are very common in veins, especially in the veins where the blood transported to the heart against gravity. They are absent in the vena cava, portal, uterine, ovarian, and hepatic veins. The pelvic veins are devoid of valves and have a great tendency to form plexuses called valveless venous system (VVS). It has long been known that a percentage of cases of chronic empyema cause brain abscess by the lodgment of septic emboli. Likewise, on occasional instances the carcinoma of the prostate; the vertebral column riddled with secondaries due to similar spread. These and some other metastases in which the vascular pathway of spread has been obscure are explainable by an appreciation of the anatomy of the valveless vertebral and cranial venous system (VVS, CVVS).

There are several plexuses of thin-walled valveless veins in relation to the vertebral bodies (see FIGS. 22, 23). The external vertebral venous plexus (VVS) consists of anterior vessels in front of the vertebral bodies, and a posterior one on the back of the arches of the vertebrae and in the adjacent muscles. The internal vertebral plexus consists of post central portion and a prelaminar; each of these sections drained by two vertical vessels. All these plexuses are in free intercommunication with each other and receive the basivertebral veins draining the bodies of the vertebra (FIGS. 22, 23, 24, 25, 26). The inter-vertebral veins; which pass through the inter-vertebral foramina with the spinal nerves and arachnoid villi (FIG. 26) drain them to and from the SAS CSF. The veins of the arachnoid villi also drain the spinal cord. These valveless veins are the veins which are connected to the arachnoid villi (FIGS. 24,25,26) on the nerve root that transfers the therapeutic agents injected perispinal into CSF in the SAS describe by Shantha (Shantha T R and Evans J A: Arachnoid Villi in the Spinal Cord, and Their Relationship to Epidural Anesthesia. Anesthesiology 37:543-557, 1972). These segmental inter-vertebral veins pour their blood into vertebral, intercostal, lumbar, and lateral sacral veins. They also communicate with veins of the portal system.

It is apparent that coughing, straining at stool evacuation, and such physical forces increases the intra abdominal-thoracic-pelvic pressure that may dislodge tumor cells or infected emboli from Systemic or portal areas into the vertebral venous system. Once dislodged, they may gain lodgments in vertebrae, spinal cord, skull, or brain. That is why the spread of liquid injected epi, inter, perispinally or epidurally at the cervical region does not gain access to the brain through the VVS, as published by some researchers, because, unlike tumors and infected emboli, they do not form emboli. The liquid therapeutic agents gets dissipated rapidly from the injection site for transport to the brain. Their spread to the spinal cord and brain is through the CSF from SAS as described above (FIGS. 23, 24, 25, 26, 27).

These CVVS and VVS veins are functionally separate from the systemic venous system in that they do not have any valves, and the blood can flow in both directions from upper olfactory mucosa and eyes to inside brain vascular system and vice versa. These CVVS conduct the therapeutic agents to the Brain bypassing capillary BBB (FIGS. 22, 23, FIG. 19 #365). Batson, in 1940, proposed that this vertebral venous plexus provided the route by which prostate cancer metastasizes to the vertebral column, now known as Batson's vertebral venous Plexus (Batson O V. The function of the vertebral veins and their role in the spread of metastases. Ann Surg 1940:112:138-149. Batson O V. The vertebral vein system. AJ Radiology 1957, 78:195-212. Anderson R. Diodrast studies of the vertebral and cranial venous systems. J Neurosurg 1951:8:411-422). Even though widely valued as a possible route by which cancer cells may spread to the spine there has been hardly any report that CVVS plexus may play an important role in delivery of therapeutic agents from the olfactory area of the nose (OAN-ORE), sphenoid sinus, sphenopalatine ganglion, sinuses and recesses in the wall of the nose, and eyeballs to the CNS by passing BBB as described here.

This CVVS also plays a role in the transport of therapeutic agents in addition to other neuronal structures described herein. The use of CVVS-VVS Batson's plexus is anatomically and physiologically distinct from the other systemic venous system. It acts as a route of delivery of therapeutic, pharmaceutical, biochemical, and biological agents or compounds for clinical use, and as a route for delivery of large molecules to the brain, spinal cord, eyes, inner ear, and the cranial nerves. This description in this patent is a continuation to the methods of use of olfactory area of the nose to deliver therapeutic molecules to the nervous system. It is the retrograde flow of valve less veins described herein that plays a role in the therapeutic agents' transport to the brain and the spinal cord fron the olfactory nasal area and the eyeballs (FIGS. 22, 23).

In non-brain systemic capillaries, therapeutic agents and compounds having molecular weights greater than 25,000 Daltons are easily transported across the endothelial wall. As described above, the endothelial cells in 400-mile long brain capillaries are tightly packed with encasing of glial cells feet, creating a BBB that blocks the passage of most molecules with MW greater than 600 Daltons. The blood-brain barrier blocks a good number of molecules except those that cross cell membranes by means of lipid solubility such as, oxygen, carbon dioxide, and ethanol; and those which are allowed in by specific transport systems, for example: sugars, amino acid-proteins complexes (insulin, IGF-1), purines, nucleosides, and organic acids. The substances having a molecular weight greater than 600 daltons cannot cross the blood-brain barrier, whereas substances having a molecular weight less than 600 daltons can cross the blood brain barrier. The valveless CVVS vertebral venous system (VVS) can deliver the compounds with MW greater than 2000, up to 150,000 such as Etanercept and other monoclonal antibodies as described in U.S. Pat. No. 7,214,658 B2, U.S. Pat. No. 6,982,089 B2, Patent Application Publication Number: 2007/0196375 A1, and U.S. Pat. No. 8,119,127 B2. Hence, in the olfactory nasal area (ORE) and CVVS venous system communications with CNS play a role in the transport of large MW therapeutic agents to the CNS to treat Alzheimer's and other neurodegenerative diseases. With electroporation and Iontophoresis application described in this invention, it is possible clinically transport large molecules of therapeutic agents to the CNS to treat Alzheimer's and other neurodegenerative diseases.

The Therapeutic Agents of this Invention Delivered by Iontophoresis Electrical Stimulator-Catheter System Through Ore, CVVS, Sphenoid Sinus, and Circumventricular Organs of this Invention to Treat Alzheimer's Disease Described Herein.

I. Glutamate receptor antagonist: NMDA-receptor blocker ketamine to prevent the glutamate mediated excitotoxicity damage of neurons and glia in Alzheimer's disease

II. β-amyloid inhibitor or clearer from the CNS in Alzheimer's disease: bexarotene which increases the production of a fat-protein complex apolipoprotein E, that helps to clear excess β amyloid form the neuropil of the brain and enhances the phagocytosis of the Aβ,

III. Insulin, to augment and amplify the effects of other therapeutic agents such as bexarotene, IGF-1, AChEIs, Etanercept, Bevacizumab, solanezumab, and as anti Alzheimer's disease effects on its own right by improving the memory and cognition,

IV. Insulin like growth factors (IGF-1), as a neurotrophic factor to prevent apoptosis, preserves the function of the remaining neurons, and glial cells, as well as maintains the integrity of nerve fibers (white mater of the brain) and their synaptic junctions.

V. Acetylcholine esterase inhibitors (AChEIs) to increase the acetylcholine content of the neurons that have lost it or have less of it to restore memory and cognition of the CNS.

VI. Monoclonal antibodies such as Etanercept, Bevacizumab, solanezumab to reduce the inflammatory process and autoimmune response involved in the etiology of the Alzheimer's disease. It is important to note that we use multiple therapeutic agents to treat this dreaded disease as described in this invention. A single agent can treat only one component of the Alzheimer's disease or neurodegenerative diseases, but multiple compounded combined therapeutic agents as described in this invention will have far-reaching effect in curtailing, slowing down and curing Alzheimer's, and other diseases of the CNS.

The Advantages of Olfactory Region, Sphenopalatine Ganglion, and Trigeminal Nerve Delivery of Insulin, Bexarotene, Ketamine, Monoclonal Antibodies, IGF-1, and Cholinesterase Inhibitor Therapeutic Agents for the Treatment of Alzheimer and Related Diseases as Described in this Invention

This present invention is a method of use of electrical impulses to create electroporation and lontophoresis through the above-described anatomical regions to transmit and transport large MW therapeutic agents to the CNS to cure and/or curtail Alzheimer's disease and related diseases by passing BBB. These regions are used for administration of insulin, IGF-1 protein neurotrophic factor, vitamin A related compound bexarotene to remove B amyloid, monoclonal antibodies to reduce inflammation, acetylcholine esterase inhibitors to enhance the acetylcholine content of the brain, and ketamine as NMDA excitotoxicity blocker, which are combined as needed therapeutic agents to improve the CNS function, cure or curtail Alzheimer's disease. Various adjuvant pharmaceutical, biochemical, nurticeuticals, and biological agents or compounds have been developed or are being developed to treat Alzheimer's and neurodegenerative diseases in conjunction with the above-described therapeutic agents. They can be used by using the method described herein. The advantages of these inventive therapeutic agents uses to treat Alzheimer's disease using the Olfactory nerve-mucosal region (ORE) are as follows:

a) Due to the close proximity of the olfactory nerves, sphenopalatine ganglion and its branches, and trigeminal nerves, pituitary gland, hypothalamus, it is easy to stimulate the central nervous system by transmitting lontophoresis electrical impulses (FIGS. 1-5, 10,11) and deliver therapeutic agents through these neural pathways;
b) Ease and convenience: This method is easy to use, painless, and does not require strict sterile technique, intravenous catheters or other invasive devices;
c) It is immediately and readily available to all patients at all times;
d) High therapeutic efficacy: Due to the achievement of higher local concentration of therapeutic agents in the CNS through the rich nerve plexus and CVVV delivered to disease afflicted areas of the CNS;
e) Increased efficacy of its use along with adjuvant therapeutic agents: Due to the ability of the administered therapeutic molecule to be bioavailable so that it reaches the target tissue without the degradation caused by digestive enzymes, hepatic or systemic circulation (first phase metabolism); and the ability of the insulin to augment and amplify the effects of other therapeutic agents used to treat CNS disease;
f) Fast onset of action: Due to their proximity to the CNS, the site where they are needed, most of the therapeutic modalities reach the CNS within seconds to a few minutes;
a) Fewer side effects: Due to lower required dosage to attain the therapeutic effectiveness due to use of insulin which has a augmentative and amplifying effect on adjuvant therapeutic agents;
b) The inventive device used for long duration. Iontophoresis and/or electroporation enhance the uptake of large molecular weight therapeutic agents for prolonged periods.
c) It is a low cost, patient and healthcare provider friendly, hardly invasive, non injectable, and safe method when used appropriately; and;
d) Electrical impulses act as iontophoresis, of the olfactory mucosa, sphenopalatine ganglion and sphenoid sinus lining (FIG. 17), thus augmenting the uptake of therapeutic agents from these regions to be delivered to the CNS by passing the BBB in the treatment of Alzheimer's and other neurological diseases.
e) Rich network of valveless CVVS (FIGS. 22, 23) and circumventricular organs (FIG. 15) described here also facilitates the delivery of therapeutic agents to the brain bypassing the arterial BBB.
f) Once the therapeutic agents enter the SAS, CSF, CVVD, VVS, circumventricular organs, Virchow-Robin space, and cerebral circulation as described above, they are distributed all over the brain and enter the neuropil at Circumventricular organs (FIG. 15), which does not have classic BBB seen in intra-cerebral BV.

Use of olfactory mucosal route to deliver therapeutic agents may have effect on smell (anosmia). Nasal congestion due to cold or allergies, sinus pathology, tumors, and nasal septal diseases may interfere with the introduction of device, but are not contraindications to use this inventive device to treat Alzheimer's disease.

Insulin to Treat Alzheimer's Disease and to Augment and Amplify the Effects of Other Therapeutic Agents Described in this Invention

The novel studies by Havrankova et. al. showed the presence of insulin receptors widely distributed in the brain which are identical to insulin receptors elsewhere. They also showed insulin in the brain at concentration 10-100 times higher than insulin levels in the plasma. Their Immunofluorescent studies showed the insulin to be present within nerve cell bodies and synapses. They also discovered that the insulin receptors in the brain were unchanged in diabetes induced test animals. These findings indicating that the hormone and its receptor in the central nervous system (CNS) are independent of factors that regulate their counterparts in the periphery (Havrankova, J., J. Roth, and M. Brownstein. 1978. Insulin receptors are widely distributed in the central nervous system of the rat. Nature (Lond.). 272: 827-829. Havrankova, J., D. Schmechel, J. Roth, and M. Brownstein. 1978. Identification of insulin in rat brain. Proc. Natl. Acad. Sci. U.S.A. 75: 5737-5741. Havrankova, J., J. Roth, and M. Brownstein. 1979. The American Society for Clinical Investigation, Volume 64 August 1979 636-642).

Where this high insulin arises in the brain is not clear. Certainly, it is not from CSF, because the concentration of insulin in the cerebrospinal fluid is only about 25% of the plasma level and its volume, relative to the brain volume, is very small. It is possible that pancreatic insulin present in the plasma and cerebrospinal fluid is taken up and stored by cells in the brain? However, the BBB has been found to be slowly and incompletely permeable to circulating insulin so that the brain tissue would need an active transport and concentration mechanism to build up the levels of insulin that they detected. Insulin receptors, which are widespread in the rat brain, can assume the role of a concentrating system, and their findings can be partially explained by the insulin present on the receptors. However, there is no close correlation between the insulin content and the receptor content in different regions that were examined. Alternatively, insulin might be synthesized by cells in the central nervous system. The finding of insulin within immature nerve cell bodies by immunocytochemistry is to some extent in favor of the local synthesis of insulin. These preliminary data, in addition to strengthening the possibility that brain insulin is produced locally, suggest that insulin and insulin receptors in the central nervous system are regulated independently of their counterparts outside the nervous system. Their degradation occurs in Alzheimer's disease; hence, ORE administration of insulin as described in this invention will restore insulin levels in the brain to maintain the structure and function of nerve tissue.

Then what is the role of insulin in the central nervous system?

a) Insulin is known to affect glucose metabolism in the central nervous system and directly affects glucose oxidation and glycogen synthesis.
b) Insulin is a mitogen: Hence, it is a fetal growth promoter in the brain and other parts of the body,
c) Insulin is important during growth, maturation, and myelination of the central nervous system.
d) Evidence suggests insulin receptors are present on synaptosomes; it is possible that insulin plays a role in neurotransmission, either as a neurotransmitter itself or as a neuro-modulator and/or participates in production and liberation of neurotransmitter especially acetylcholine (AChE) needed for memory and cognition.
e) It has been known for some time that exogenously administered insulin into the carotid artery and ventricles can induce some effect within the central nervous system that eventually results in neurally mediated peripheral hypoglycemia (Szabo, O., and A. J. Szabo. 1972. Evidence for an insulin sensitive receptor in the central nervous system. Am. J. Physiol. 223: 1349-1353. Szabo, O., and A. J. Szabo. 1975. Studies on the nature and mode of action of the insulin-sensitive glucoregulator receptor in the central nervous system. Diabetes. 24: 328-336. Woods, S. C., and D. Porte, Jr. 1975. The effect of intracisternal insulin on plasma glucose and insulin in the dog. Diabetes. 24: 905-909). It may be that this effect is produced by insulin acting through receptors on specific central cells.
f) The list of peptides identified in both the central nervous system and the gastrointestinal tract is rapidly expanding. Insulin, like some of the gastrointestinal peptides such as somatostatin, vasoactive intestinal polypeptide, and thyrotrophic-releasing hormones are included among the putative neuro regulators (Barchas, J. D., Akil, H., Elliott, G. R., Holman, R. B. & Watson, S. J. (1978) Science 200, 964-973).
g) Because insulin and insulin receptors are ubiquitous (ever-present, found everywhere in the brain) throughout the central nervous system, we anticipate an extensive physiological role for insulin in the CNS system much more so than the non-nervous tissue.
h) Preliminary studies show that genetically obese diabetic mice, in which circulating insulin is markedly increased and insulin receptors in liver, fat, and other tissues are severely decreased, have normal concentrations of insulin and of insulin receptors in the brain, which indicates how important it is to have normal insulin in the brain to prevent Alzheimer's and other neurological diseases.
i) In related studies, in rats rendered hypoinsulinemic and diabetic by treatment with streptozotocin there was no decrease in the concentration of brain insulin. These preliminary data, in addition to strengthening the possibility that brain insulin is produced locally, suggest that insulin and insulin receptors in the central nervous system are regulated independently of their counterparts outside the nervous system and play an important role in Alzheimer's disease production and its treatment.

The body has extraordinary mechanisms for transporting insulin from plasma to brain, concentrating it, and maintaining constant levels despite extreme changes in the availability of plasma insulin, or, much more likely, that brain insulin is produced within the brain. Given the widespread distribution of high levels of insulin and its receptors in the brain which are unchanging in response to major metabolic events, it is clear that the insulin plays an important role within the CNS as neurotransmitter, neuromodulator, or a growth factor at all levels of the CNS whose decline results in multiple neurodegenerative diseases including psychological illnesses.

It is worthy to note that insulin has been investigated in the treatment of Alzheimer's for more than a decade as noted in these publications (U.S. Pat. No. 6,313,093 B1, Steen E, Terry B M, Rivera E J; et al. Impaired insulin and insulin-like growth factor expression and signaling mechanisms in Alzheimer's disease—is this type 3 diabetes? J Alzheimer's Dis. 2005; 7(1):63-80, Thorne R G, Pronk G J, Padmanabhan V, Frey W H 2nd: Delivery of insulin-like growth factor-I to the rat brain and spinal cord along olfactory and trigeminal pathways following intranasal administration. Neuroscience 2004, 127:481-496. Matsuoka Y, Gray A J, Hirata-Fukae C, Minami 55, Waterhouse E G, Mattson M P, LaFerla F M, Gozes I, Aisen P S: Intranasal NAP administration reduces accumulation of amyloid peptide and tau hyperphosphorylation in a transgenic mouse model of Alzheimer's disease at early pathological stage.) Mol Neurosci 2007, 31:165-170. Teen E, Terry B M, Rivera E J, Cannon J L, Neely T R, Tavares R, Xu X J, Wands J R, de la Monte S M: Impaired insulin and insulin-like growth factor expression and signaling mechanisms in Alzheimer's disease: is this type 3 diabetes? Alzheimers Dis 2005, 7:63-80. Reger M A, Watson G S, Frey W H 2nd, Baker L O, Cholerton B, Keeling M L, Belongia O A, Fishel M A, Plymate S R, Schellenberg G O, Cherrier M M, Craft S: Effects of intranasal insulin on cognition in Memory-impaired older adults: modulation by APOE genotype. Neurobiol Aging 2006, 27:451-458. Reger M A, Watson G S, Green P S, Baker L O, Cholerton B, Fishel M A, Plymate S R, Cherrier M M, Schellenberg G O, Frey W H 2nd, Craft S: Intranasal insulin administration dose-dependently modulates verbal memory and plasma amyloid-beta in memory-impaired older adults. Alzheimers Dis 2008, 13:323-331). Nevertheless, insulin has never been used with other therapeutic agents as described in this invention to treat Alzheimer's disease to augment and amplify the effects of adjuvant therapeutic agents to cure or curtain Alzheimer's disease. We are of the firm belief that multiple therapeutic agents needed to treat Alzheimer's disease as shown in AIDS and treatment of many incurable diseases including cancers. These above reports also fail to recognize the immense effectiveness of insulin to augment and amplify effects on other therapeutic agents on the neuropil with embedded with neurons used to treat Alzheimer's disease besides using insulin alone. This physiological effect lowers the dose of other therapeutic agents, thus lowering the adverse effect of these therapeutic agents, at the same time reducing the cost of the therapy.

Suzanne Marie de la Monte, Jack Raymond U.S. Pat. No. 7,833,513 B2 describe in their invention methods for diagnosing Alzheimer's disease by determining the level or function of insulin, insulin-like growth factors, and their receptors. The invention further relates to methods for the treatment of AD by administering an insulin agonist and an insulin-like growth factor agonist. The insulin agonist and the IGF agonist administered in appropriate manner, e.g., intraventricularly (e.g., with an intraventricular stent), intracranially, intraperitoneally, intravenously, intra-arterial, nasally, or orally. These studies do not describe the method of delivering to the olfactory mucosal region (ORE) exclusively and the lontophoresis method for enhancing its uptake with a special delivery catheter delivered to the ORE, trigeminal and other cranial nerves, CVVS, sub Perineural epithelial, and nerve fascicular interstitial spaces, sphenoid sinus, and circumventricular organs delivery and their SAS-CSF spread to the brain. They also do not use the combination of multiple compounded therapeutic agents we describe in this invention to treat the complex AD disease as a whole. They also do not describe the augmentation and amplification effects of insulin on other therapeutic agents. Our experience shows that the administration of the insulin other than nasal olfactory mucosal routes to deliver to the CNS is not practical and results in hypoglycemia. Intraventricular administration is not possible in humans, fraught with complications, and expenses. It applies to test its effect only in experimental animals.

William H. Frey, II, U.S. Pat. No. 6,313,093 HI, disclosed a method for transporting neurologic therapeutic agents to the brain by means of the olfactory neural pathway and a pharmaceutical composition useful in the treatment of brain disorders. They never emphasized its deposition on the olfactory mucosal lining only for maximum delivery, and they did not describe any method of delivery to the olfactory mucosa or uptake enhancer as we describe here in using lontophoresis. They indicate a neurologic agent may be administered intranasally as a powder, spray, gel, ointment, infusion, injection, or drops. We were unable to deliver effective doses of insulin in any other form than instillation on the olfactory mucosa in liquid form. Injection into nasal cavity is no different from subcutaneous injection, and will not reach the CNS in the concentration needed for the treatment of Alzheimer's disease by passing the BBB. They do not use other therapeutic agents in combination to treat Alzheimer's disease, its pathology, neurotransmitters, and its etiology as described in this invention here, such as insulin, bexarotene, and ketamine, monoclonal antibodies, IGF-1, and cholinesterase inhibitor therapeutic agents.

None of these studies describes the various mechanisms involved in the transport of therapeutic agents to treat Alzheimer's disease. Such delivery mechanisms involve cranial vertebral venous system (CVVS), sub Perineural epithelial, and nerve fascicular interstitial spaces, Virchow-Robin spaces, SAS, CSF, and circumventricular organs (FIGS. 10-23) spread; which all play an important role as described in this invention for the treatment of Alzheimer's disease and other neurodegenerative diseases of the CNS. Olfactory mucosal, olfactory Transneuronal antegrade and retrograde transport of the neurologic agent entering through the peripheral olfactory neurons system, meaning axons of the olfactory nerves to the brain as noted in many of the articles and patents, plays a minor role in spread of therapeutic agents. When the therapeutic agents enter the axon of the peripheral olfactory neurons, they have to pass through the complex synaptic golemrular masses in the olfactory bulb, which poses a formidable obstacle, and slows or may even block the passage further to a trickle. It is the sub Perineural epithelial, and nerve fascicular interstitial spaces of the axonal fasciculi, which spread (see FIGS. 10-13, 16-19) the therapeutic agents to the SAS and CSF, then into the brain, that is responsible for the therapeutic agents' spread to cure and curtail the Alzheimer's disease as described in this invention.

The latest study by Craft and her associates (2011) whose findings are incorporated herein showed that insulin has a number of important functions in the central nervous system and plays a role in Alzheimer's to improve memory and cognition as observed by many other investigators (Craft S. et al. Intranasal Insulin Therapy for Alzheimer Disease and Amnestic Mild Cognitive Impairment. Arch Neurol. published online Sep. 12, 2011, Pages 1-13). There have been numerous reports besides our own unpublished findings even before this study on the effect of insulin as noted in the above reference and this is not a new finding as touted in the news media.

The role of insulin in the central nervous system is becoming clear from the above discussion. It plays role in memory and cognition, and reduced insulin found in Alzheimer's disease brain. Insulin plays a role in development of the CNS, and responsible for maintaining the functioning of the neurons, their fibers and synapses, as well as neurotransmitters such as acetylcholine, which are disrupted in Alzheimer's disease. Brain insulin receptors are densely localized and concentrated in the hippocampus, the entorhinal cortex (olfactory bulb connected), and the frontal cortex. These insulin receptors found primarily in synapses, where insulin signaling contributes to synaptogenesis and synaptic remodeling (Chiu S L, Chen C M, Cline H T. Insulin receptor signaling regulates synapse number, dendritic plasticity, and circuit function in vivo. Neuron. 2008; 58 (5):708-719. Zhao W Q, Townsend M. Insulin resistance, and myloidogenesis as common molecular foundation for type 2 diabetes and Alzheimer's disease. Biochim Biophys Acta. 2009; 1792 (5):482-496). Insulin also modulates glucose utilization in the hippocampus and other brain regions as it does in the rest of the body and facilitates memory at optimal levels in normal metabolism. The importance of insulin in normal brain function is underscored by evidence that insulin dysregulation contributes to the pathophysiology of Alzheimer disease (AD), a disorder characterized in its earliest stages by synaptic loss and memory impairment possibly associated with decline of acetylcholine. Our study on people with impaired cognition showed insulin, monoclonal antibodies, and ketamine delivery through the ORE resulted in rapid recovery of cognition and enhanced memory. This therapy also helped many patients with depression due to Lyme diseases, psychological depression, depression due to neurodegenerative diseases, cancers, and other such conditions to recover rapidly. Our trial studies showed that the Insulin with Progesterone, ketamine, and monoclonal antibodies administered to ORE is very effective in the treatment of PTSD, stroke, concussion, and traumatic brain injury.

Studies show that the Insulin levels and insulin activity in the central nervous system reduced in AD. Insulin has a close relationship with the β-amyloid peptide, a toxic peptide produced by endoproteolytic cleavage of the amyloid precursor protein. Insoluble Aβ deposits in the brain's parenchyma and vasculature in Alzheimer's is an important pathology found in Alzheimer's disease. Soluble Aβ species, particularly oligomers of the 42 amino acid species (Aβ42), also have synaptotoxic effects (Selkoe D. J. Soluble oligomers of the amyloid beta-protein impair synaptic plasticity and behavior. Behav Brain Res. 2008; 192 (1):106-113). Insulin can counteract these toxic effects at synapses and promote synaptogenesis.

We believe that bexarotene (the latest drug involved in treatment of Alzheimer's disease) along with insulin acts by removing the soluble Aβ species, particularly oligomers of the 42 amino acid species (Aβ42), which have synaptotoxic effects (insulin counteracts its effects) and improve the memory. Our invention of using Insulin with bexarotene will augment and amplify the effects of bexarotene and at the same time reduce the excitotoxic effects of glutamate (due to ketamine), make easier to synthesize glutathione, which is neuroprotective, and facilitate the removal of the ROS. Its effects further augmented by insulin administered to olfactory mucosa, olfactory nerves, trigeminal nerves, and sphenopalatine ganglion. Insulin modulates the levels of Aβ and protects against the detrimental effects of Aβ oligomers on synapses. Thus, reduced levels of insulin and of insulin activity contribute to a number of pathological processes that characterize Alzheimer's disease, which are ameliorated by administration of multiple specific therapeutic agents described here. Restoring insulin to normal levels in the brain with other Alzheimer's disease therapeutic agents described here provides therapeutic benefit to the patients with Alzheimer's disease and other degenerative brain afflictions.

Parenteral (Subcutaneous and IV) administration of insulin is not possible for the treatment of Alzheimer's disease owing to the risk of hypoglycemia or induction and/or exacerbation of peripheral insulin resistance and the presence of BBB. In contrast, intranasal olfactory neuronal mucosal administration of insulin provides a rapid delivery of insulin to the central nervous system via bulk flow along olfactory and trigeminal subperineural epithelial spaces (FIGS. 15, 17) to the SAS of the CNS, CSF, circumventricular organs, CVVS, and then distributed to the rest of the brain (Shantha T. R. and Yasuo Nakajima. Histological and Histochemical Studies on the Rhesus Monkey (Macaca Mulatta) Olfactory Mucosa. Yerkes Regional Primate Research Center, Emory University, Atlanta, Ga.: Z. Zellforsch. 103, 291-319 (1970). Shantha T R and Evans J A: Arachnoid Villi in the Spinal Cord, and Their Relationship to Epidural Anesthesia. Anesthesiology 37:543-557, 1972. Shantha T R: Peri-vascular (Virchow-Robin) space in the peripheral nerves and its role in spread of local anesthetics, ASRA Congress at Tampa, Regional Anesthesia 17 (March-April, 1992). Shantha T R and Bourne G H: The “Perineural Epithelium”: A new concept. Its role in the integrity of the peripheral nervous system. In Structure and Function of Nervous Tissues. Volume I. pp 379-458. (G H Bourne, Ed.). Academic Press, New York. 1969. U.S. Patent Application Publication Number: 201110020279 AD, Rabies cure—Shantha).

The delivery of inhalation method through the nose or mouth to treat diabetes avoided due to its mitotic effect that can result in cancers of the lungs as reported. Pfizer reported six cases of lung cancer after its use and withdrew the drug Exubera™ (inhalation insulin trade name) from the market (Shantha; TR: Unknown Health Risks of Inhaled Insulin. Life extension September 2007, Page 78-82). This publication resulted in withdrawal of Exubera by Pfizer and saved thousands of patients from future lung cancer using this method of insulin delivery. Letter: comment by the editors of the Life extension on Dr. Shantha's research findings: Inhaled Insulin Increases Lung Cancer Risk. Life Extension September 2008, Page 20. T. R. Shantha, Jessica G. Shantha: Inhalation Insulin and Oral and Nasal Insulin Sprays for Diabetics: Panacea or Evolving Future Health Disaster: Part 1; Townsend Letter—January 2008; Pages 94-98. T. R. Shantha, Jessica G. Shantha: Inhalation Insulin and Oral and Nasal Insulin Sprays for Diabetics: Panacea or Evolving Future Health Disaster: Part 2 Townsend Letter—January 2009; Pages 136-140). ORE delivery of insulin has no such adverse effects, though we avoid its use in patients with nasal polyps and tumors. The delivery of therapeutic agents including insulin is a slower delivery via olfactory nerve other cranial nerve axoplasm-olfactory bulb axonal transport. On the other hand, it rapidly reaches through the sub Perineural epithelial, and nerve fascicular interstitial spaces of the olfactory nerve, cranial nerve 1-VI, and sphenopalatine ganglion nerves (see FIGS. 10-12, 16-18) as well as CVVS to reach the CNS and CVO through SAS CSF and nerve fasciculi. Olfactory nerve and olfactory mucosal delivery will not adversely affect blood insulin or glucose levels unless delivered to the respiratory mucosa of the nasal cavity. That is one of the reasons the special therapeutic agents' delivery catheters designed in this invention to prevent systemic spread. In rodent models, intranasally administered insulin binds to receptors in the hippocampus and the frontal cortex within 60 minutes, which is similar in vertebrates including humans.

In human studies, olfactory mucosal intranasal (olfactory mucosa nerves-ORE) insulin increases insulin levels in cerebrospinal fluid (CSF) within 60 minutes, and acutely enhances memory. Such a fast spread is not possible through axoplasm spread of peripheral olfactory neurons and other cranial nerve fasciculi. It is possible only through the sub Perineural epithelial interstial nerve fasciculi spread. Furthermore, a 3-week trial by Craft et al. of daily administration of intranasal insulin improved delayed story recall and caregiver-rated functional status in a small sample of adults with AD and in adults with amnestic mild cognitive impairment (aMCI), a condition thought to represent a prodromal (premonitory, the early stage) symptom of AD in most cases. Insulin improves memory in normal adults and patients with Alzheimer's disease without altering blood glucose. This is a known fact. But in other combinations therapeutic agents with insulin will have a profound effect on the treatment of Alzheimer's disease and other neurodegenerative diseases.

In a study of students taking the comprehensive examination and test, we prescribed 2-4 IU insulin instilled on each sides of the nose delivered to the ORE in supine position, 1-2 hours before the examination using our delivery device. The students who used the insulin delivered to the ORE scored higher in test scores. They reported better and more rapid recall from memory during taking the test to answer the questions, compared to the controls. We are planning an expanded study and to analyze statistically to report its positive significance and its use in public speakers (T. R. Shantha, use of olfactory mucosal insulin to enhance the memory and recall before student examinations, written tests and before presentations in front of audience at meetings. Expanded study-under preparation).

It is important to note that we treated many cases of PTSD, concussion, brain injury, stroke victims who demonstrated memory loss like Alzheimer's disease using ORE delivery of insulin, monoclonal antibodies, IGF-1, Ketamine, and progesterone, every day for one week, then every other day for one week and then twice weekly for week, and then weekly. We also provided the home therapy kit with these therapeutic agents and trained them in the method of delivery to ORE, so that they can administer on their own. Some of the patients received hyperbaric oxygen therapy also. The results were dramatic. One of the stroke patient, who could not talk, started talking within one week of treatment. In general, the research shows that in the brain and spinal cord, progesterone with insulin (and other therapeutic agents such as NMDA blockers and monoclonal antibodies) protect and rebuild the blood-brain barrier (BBB) with improvement of vascular tone. It also said to reduce cerebral edema, down-regulate the inflammatory cascade (cytokine generation), reduce excitotoxicity of glutamate, and seizure activity, stimulate myelination in damaged axons by oligodendroglia, and decrease apoptosis of neurons.

The ORE use of progesterone based on its neuroprotective-neurotrophic effects. Progesterone neurotrophic effects are augmented and amplified by the Insulin, and IGF-1. Memory function is improved by cholinesterase inhibitors. Glutamate excitotoxic effect and depression are reduced by Ketamin or other NMDA antagonists, and the inflammatory cytokines is countered by monoclonal antibodies (Etanercept—a potent anti-TNF fusion protein). Every patient with PTSD should be treated with a combination of these therapeutic agents described herein, not just a single agent like progesterone. The results were dramatic. All these patients also received magnesium L-threonate, and Zinc orally with vitamin B₁, B₁₂ injections or through the intranasal ORE administration with high oral dose of vitamin B complex D₃.

Energy metabolism in the CNS is dependent upon glucose uptake, and regulated by insulin in key brain regions, which are part of memory and cognition. It known that glucose uptake and utilization are deficient in patients with Alzheimer's disease. It is likely that improved memory and test scores in these test taking students after insulin ORE delivery is due to augmented production of ATP by aerobic glucose metabolism, increased neurotrophic protein production and its activity, amplified acetylcholine output and activity at synapses and their utilization related to recall in reminiscence related brain regions. All these metabolic effects of insulin enhance memory and recall.

Recent studies show that the gene expression levels of insulin, IGF-1, and their receptors are markedly reduced in the brains of patients with Alzheimer's disease. Consequently, the ability to deliver insulin and IGF-1 to the CNS without altering blood glucose and enhancing protein synthesis could provide an effective means to improve glucose uptake and utilization, improve neuronal function by producing intracellular neurotrophic factors, and reduce cognitive deficits in patients with memory disorders as seen in Alzheimer's disease. Longer treatment with olfactory mucosal insulin (21 days) in the Craft et al. study enhanced memory, attention, and functioning compared with placebo in patients with either early stage Alzheimer's disease or mild cognitive impairment. They never used other therapeutic agents including IGF-1, which we describe in this inventive method to treat Alzheimer's comprehensively. Alzheimer's disease is a complex disease, and hence needs a combination of therapeutic agents to attack the underlying pathology. Combining the other therapeutic agents described in this invention even further enhances the therapeutic effect in improving the signs and symptoms of Alzheimer's and other neurodegenerative dementia related to aging and other related pathology. It is a known fact that the combination composition of compounded therapeutic agents has a synergic effect in the treatment of many diseases, including AIDS (used now), cancers, and other neurodegenerative diseases (Shantha-unpublished data).

Further, the insulin augments and amplifies the effect of many therapeutic agents such as bexarotene, acetylcholine, monoclonal antibodies, and ketamine many fold. Alabastor et al discovered such an effect. Their studies showed that the insulin enhances the activity of other therapeutic agents such as methotrexate 10,000 times (Oliver Alabaster′ et al. Metabolic Modification by Insulin Enhances Methotrexate Cytotoxicity in MCF-7 Human Breast Cancer Cells, Eur j Cancer Clinic; 1981, Vol 17, ppl 223-1228).

In our decade of studies, such an effect found when insulin used with other therapeutic agents to treat cancers, chronic infections, Methicillin-resistant Staphylococcus aureus (MRSA) infection, and other afflictions locally or systemically. Donato Perez Garcia first reported insulin augmentation of anti-spirochetal effect chemotherapeutic agents, in his U.S. Pat. No. 2,145,869 for the treatment of neuro syphilis. We have used insulin delivered to the olfactory mucosa for the treatment of many neurodegenerative diseases including the cases of reduced mental cognition with declining memory in the aged, Parkinson's with glutathione, as well as for depression due to any number of reasons including PTSD, concussions, cancers, Lyme disease, strokes etc. It reduced the depression, improved the memory, and increased cognition. IGF-1 as a neurotophic factor for the treatment of Alzheimer's disease: It is important to note that the Insulin-like growth factor-1 (IGF-1: 7.65 kDa) and insulin have similar three-dimensional structures and a similar function and it is a single-chain polypeptide of 70 amino acids. IGF-1 is a trophic factor that circulates at high levels in the blood stream. It is a much more effective neurotrophic factor compared to insulin and has a positive effect on the neurons in repairing and maintaining functioning in the brain in Alzheimer's disease. The insulin augments and amplifies the effects of IGF-1 in the neurons. The IGF-1 influences neuronal structure and functions throughout the life span. Studies have shown the effect of IGF-1 on the hair cell growth of the inner ear. The IGF-1 has the ability to preserve nerve cell function specially neurons and promote nerve growth in experimental studies.

The IGF-1 and insulin play an important role in maintaining proper integrity, growth, repair, and functioning of the cells and neurons in the brain in particular. Because of these properties, recombinant human IGF-1 is in clinical trials for the treatment of amyotrophic lateral sclerosis (U.S. Patent Application Publication Number: US 2009/0105141 A1). The primary function of IGF-1 is to stimulate cell growth in every part of the body including neurons. Body builders use 100 mcg to 400 mcg per shot without concern and ill effect for its anabolic effects.

Insulin incorporated and compounded, to treat Alzheimer's disease with other therapeutic agents as described; to augment and amplify their effect besides its own effects to increase the memory and cognition. It has a trophic effect on the neurons, and it is a mitogenic. It promotes the glucose metabolism within the neuron mitochondria, which increases the ATP production aerobically. The ATP enhances the protein and peptides synthesis, and their output by the nucleus and endoplasmic reticulum by using the ATP energy provided by the mitochondria. This will enhance the protein-peptide-amino acid complexes production of every kind, including tau proteins involved in the construction and maintenance of neurotubules, neurotrophic factors, neurotransmitters, enzymes, and hormones, for example. Insulin augments the production of substrates needed to assemble neurotransmitters and protein complexes to maintain the cell wall, the integrity of the neurons, their extensions and synapses. Thus, insulin along with other therapeutic agents described in this invention prevents or delays further decay of the neurons afflicted by this disease, reduces the ROS damage to the remaining healthy nerve tissue, improves synaptogenesis, enhances the output of glutathione, and augments the production of acetylcholine and their functions, improves memory and cognition.

Besides insulin's effect on cognition and improving the psychological status of the Alzheimer's patients, its composition is used here in conjunction with the bexarotene, ketamine, monoclonal antibodies, IGF-1, and AChEIs to enhance their uptake and delivery to the CNS, as well as to augment and amplify their therapeutic effects (endocrine, paracrine and intracrine effect) on the neuropil. Besides using this for Alzheimer's disease, we have used monoclonal antibodies with insulin through ORE delivery for the treatment of many mental diseases including depression due to wide array of etiologies, autism, and Lyme disease, PTSD, and cancer patients.

The principle of this invention is to reduce the β amyloid and its soluble precursors, block glutamate damage, reduce brain inflammation, and increase the acetylcholine levels s to cure or curtail Alzheimer's symptoms and protect the neurons with the neurotrophic factors in the treatment of Alzheimer's disease and other neurodegenerative disease with herein described combination therapy. Insulin or bexarotene or other single therapeutic agents alone cannot perform all these functions, hence we have invented the use of these multiple therapeutic agents in combination to treat the underlying pathology in the CNS, and improve the brain function in totality including memory and cognition while at the same time reducing or preventing further pathological changes in the brain.

Bexarotene to Reduce Amyloid Beta (Aβ) in the Treatment of Alzheimer's Disease Using Intra Nasal Olfactory Mucosal Region Delivery Along with Insulin and Other Therapeutic Agents Described in this Invention

Neuroscientists at Case Western Reserve University School of Medicine have made a breakthrough in their efforts to find a cure for Alzheimer's disease in mice studies. The researchers' findings, published in the journal Science, show that the use of a drug bexarotene in mice appears to reverse quickly the pathological, cognitive and memory deficits caused by the onset of Alzheimer's. The results of a study led by Gary Landreth, PhD, professor of neurosciences at Case Western prompted the research. Paige Cramer, PhD candidate at Case Western Reserve School of Medicine was the first author of the study. Co-authors include John R. Cirrito, Jessica L. Restivo, Whitney D. Goebel, Washington University School of Medicine; C. Y. Daniel Lee, Colleen Karlo, Adriana E. Zinn, Brad T. Casali, of Case Western Reserve University School of Medicine, Donald A. Wilson, of New York University School of Medicine, and Michael J. James, Kurt R. Brunden, of Perelman School of Medicine, University of Pennsylvania. The research reviewed at the Eureka Alert web site on Feb. 9, 2012.

Their finding was that bexarotene therapeutic agents improve roughly 5.4 million Americans suffering from this progressive brain disease based on the mice study. Of course, the leap from mice to human clinical trials takes many years. We are all well aware that long lists of promising Alzheimer's drugs have failed in clinical trials. One of the serious problems with this drug is that it is fraught with a wide array of side effects when used systemically and it is very expensive. Our method of delivering by intranasal ORE route eliminates both these problems, and at the same time delivers the agents to the site of the disease. Further, this therapeutic agent delivered in small doses that reduce the cost and complication in the treatment of Alzheimer's disease as described including insulin in the present invention. Further, these researchers did not use insulin to augment and amplify the effects of bexarotene nor did they use ORE to deliver as we describe in this invention.

Alzheimer's disease arises from the brain's inability to clear naturally occurring amyloid beta from the brain. In 2008, researcher Dr. Gary Landreth also discovered that the main cholesterol carrier in the brain, Apolipoprotein E (ApoE), facilitated the clearance of the amyloid beta proteins. Landreth and his colleagues chose to explore the effectiveness of bexarotene, a vitamin A derivative for increasing ApoE expression. They found that the elevation of brain ApoE levels, in turn, speeds the clearance of amyloid beta from the brain. How does the Bexarotene act to clear this toxic substance? They found that it acts by stimulating retinoid X receptors (RXR), which control how much ApoE produced. The researchers were surprised to find out the speed with which bexarotene improved memory deficits and behavior as it acted to reverse the pathology of Alzheimer's disease. This study identifies a link between the primary genetic risk factor for Alzheimer's disease and a potential therapy to deal with it. Humans have three forms of ApoE: ApoE2, ApoE3, and ApoE4. It is the possession of the ApoE4 gene significantly increases the chances of developing Alzheimer's disease. Previously, the Landreth laboratory had shown that this form of ApoE was impaired in its ability to clear amyloid. The new research suggests that elevation of ApoE levels in the brain may be an effective therapeutic strategy to clear the forms of amyloid associated with impaired memory and cognition. The present view of the pool of scientists is that diminutive soluble forms of amyloid beta cause the memory impairments seen in animal models and humans with the disease. This fact is substantiated by the observation that within six hours of administering bexarotene to mouse brain, soluble amyloid levels fell by 25 percent; even more notable, the effect lasted as long as three days. Finally, this shift correlated with rapid improvement in a broad range of behaviors; by 72 hours after the bexarotene treatment, the mice began to use paper to make nests, which they were unable to do before and there was improvement in the ability to sense and respond to odors. It is oblivious that in the mice models, the Bexarotene treatment worked rapidly to stimulate the removal of amyloid plaques (soluble form?) from the brain. Research also indicates that the bexarotene reprogrammed the brain's immune cells (white blood cells, microglia) to “eat” or “phagocytose” the already formed amyloid deposits. This observation demonstrated that the drug addresses the amount of both soluble and deposited forms of amyloid beta within the brain and reverses the pathological features of the disease in mice.

Our invention of using insulin with the bexarotene with neurotrophic factor with NMDA blocker will augment such phagocytosis of the Aβ, enhance the acetylcholine, and improve the memory and cognition. Vitro studies show that the insulin augments the phagocytosis activity of the white blood cells, and increases antibody output by plasma cells. Hence, the combination of insulin and bexarotene of this invention will be of immense advantage in treating Alzheimer's disease and restore the memory and cognition to a level such that the afflicted patient can function without the help of a caregiver.

We have used insulin (with or without monoclonal antibodies) and ketamine delivered to the olfactory mucosal region (ORE) for the treatment of many neurodegenerative diseases including the cases of reduced mental cognition with declining memory in the aged, Parkinson's (with glutathione), as well as for depression due to any number of reasons including PTSD, postpartum depression, concussion, senile depression, cancer patients, stokes and Lyme disease. It reduced the depression, improved the memory, and increased cognition. Many of the psychological problems reduced or completely relieved. Further, the insulin augments and amplifies the effect of many therapeutic agents such as bexarotene, Etanercept, AChEIs, progesterone, and ketamine many fold as described in the ingenious experiments by Alabastor et al (Oliver Alabaster′ et al. Metabolic Modification by Insulin Enhances Methotrexate Cytotoxicity in MCF-7 Human Breast Cancer Cells, Eur j Cancer Clinic; 1981, Vol 17, pp 1223-1228). Insulin with IGF-1 has a trophic effect on the neurons, and it is a mitogenic, thus it prevents or delays further decay of the neurons afflicted by this disease and reduces the ROS damage to the nerve tissue that can lead to apoptosis. Besides its effect on cognition and improving the psychological status of the Alzheimer's patients, it is used in conjunction with the bexarotene to enhance its uptake and delivery to CNS, as well as to augment and amplify its effects (paracrine and intracrine effects) on the neuropil to reduce the β amyloid, and its soluble precursors, to cure and curtail the disease.

We have used bexarotene and Isotretinoin (ACCUTAIN®) to treat some cases of T-cell lymphoma and other forms of cancers. High dose usage of bexarotene in the treatment of cutaneous T-cell lymphoma is associated with hypertriglyceridemia, hypercholesterolemia and decreased high-density lipoprotein levels, as well as hypothyroidism (SI Sherman, Gopal J, Haugen B R, et al et al, and Central hypothyroidism with retinoid X receptors-selective ligands. N Engl J Med, 1999, 340:1075-1079. 7). It can also cause headache, asthenia, leucopenia, anemia, infection, rash, alopecia and photosensitivity. This is due to use of mega doses of bexarotene, 300-400 mg per m² per day for eight weeks. The manufacturer cautions that bexarotene given to diabetic patients concurrently with hypoglycemic agents may cause hypoglycemia. Our method used to deliver at the ORE is so small in dose, that it did not show any adverse reactions or complications as reported. We used bexarotene with insulin, which cut down the dose to 10-25% with similar results with none of the above-described adverse effects. The same is also true when we use it with insulin delivered to ORE to treat AD with bexarotene.

Glutamate Toxicity on Neurons and Glial Cells Contributing to Alzheimer's Disease Reduced or Eliminated by NMDA Blocker Ketamine

Glutamine (Gln), glutamate (Glu) and γ-amino butyric acid (GABA) are essential amino acids for brain metabolism and function. Astrocytic glutamine is the precursor of the important neurotransmitters: glutamate, an excitatory neurotransmitter, and GABA, an inhibitory neurotransmitter. Glutamine is a derivative of glutamic acid and its chemical name is glutamic acid 5-amide.

Reactive oxygen species (ROS) with liberation of glutamate is due to neuropil damage and apoptosis. It is a known fact that glutamate with ROS plays a major role in excitotoxicity of CNS and neuronal death due to excessive stimulation. Research shows that glutamate receptors are present in CNS glial cells as well as neurons (Steinhäuser C, Gallo V (August 1996). “News on glutamate receptors in glial cells”. Trends Neurosci. 19 (8): 339-45). The glutamate binds to the extracellular portion of the receptor and provokes a response-excitotoxicity. Overstimulation of glutamate receptors causes neurodegeneration and neuronal damage through a process called excitotoxicity. Excessive glutamate, or excitotoxins acting on the same glutamate receptors, and on over activate glutamate receptors, causes high levels of calcium ions (Ca²⁺) to influx into the postsynaptic cell. High Ca²⁺ concentrations activate a cascade of cell degradation processes involving proteases, lipases, nitric oxide synthase, and a number of enzymes that damage cell structures (ROS) often to the point of cell death (Manev H, Favaron M, Guidotti A, Costa E (July 1989). “Delayed increase of Ca²+ influx elicited by glutamate: role in neuronal death”. Mol. Pharmacol. 36 (1): 106-12). Glutamate excitotoxicity triggered by overstimulation of glutamate receptors also contributes to intracellular oxidative stress on the neurons in neurodegenerative diseases, which is restored by use of insulin and NMDA blockers as described here. Proximal glial cells use a cystine/glutamate antiporter to transport cystine into the cell and glutamate out of the cell. An excessive extracellular glutamate concentration inhibits synthesis of glutathione (GSH), an antioxidant, due to lack of enough cystine. Lack of GSH leads to more reactive oxygen species (ROS) that damage and destroy the glial cell and neurons, which then cannot reuptake and process extracellular glutamate (Markowitz A J, White M G, Kolson D L, Jordan-Sciutto K L (July 2007). “Cellular interplay between neurons and glia: toward a comprehensive mechanism for excitotoxic neuronal loss in neurodegeneration”. Cell science 4 (1): 111-146). In addition, increased Ca²+ concentrations activate nitric oxide synthase (NOS) and the over-synthesis of nitric oxide (NO). High NO concentration damages mitochondria, leading to more energy depletion, and adds oxidative stress to the neuron as NO is a ROS. In addition, cell (neuronal) death via lysis or apoptosis releases cytoplasmic glutamate outside of the ruptured cell. These two passageways of glutamate release trigger a continual domino effect of further increased extracellular glutamate concentrations and excitotoxic cell death.

Glutamate receptors significance in excitotoxicity links it to many neurodegenerative diseases including Alzheimer's disease. Glutamate is almost exclusively located inside the cells (neurons and glial cells). This is essential because glutamate receptors only activated by glutamate binding to them from the outside. Hence, glutamate is relatively inactive as long as it is intracellular.

Ketamine is one of most important NMDA blockers, thus preventing the excitotoxicity by glutamate. The micro doses of ketamine we use in the olfactory mucosal drops have no hallucinogenic or other ill effect. It is one of the ideal olfactory mucosal olfactory nerve and CVVS delivered therapeutic agents for the treatment of Alzheimer's disease, to block NMDA mediated neuronal damage. Pharmacologically, ketamine classified as an NMDA receptor antagonist. The present inventor has administered ketamine in thousands of cases as dissociative anesthetic, neuropathic pain, depression, and hiccup (Shantha, T. R. Ketamine for the Treatment of Hiccups During and Following Anesthesia: A Preliminary Report, Anesthesia, and Analgesia. Current Researches. VOL. 52, No. 5, September-October, 1973). Experiments show that it inhibits the rabies virus multiplication through this blocking mechanism (U. S. Patent Application Publication Number: U.S. Patent Application Publication Number: 2011/0020279 AD by Shantha Rabies Cure). The invention described herein incorporates ketamine delivered to olfactory mucosa, then to olfactory nerves and the CNS. It is important to note that ketamine has a mild local anesthetic effect and thus prevents the stinging/burning experienced after olfactory mucosal instillation of therapeutic agents and may lower the sensation of smell temporarily.

The invention described herein incorporates ketamine delivered to olfactory region mucosa with bexarotene and insulin and other adjuvant therapeutic agents described at present. The intranasal use of ketamine delivery to the olfactory mucosa reduced or relieved the depression associated with many neurodegenerative diseases including Alzheimer's disease, cancer patients, senility, Lyme disease, neuropathic pain, reflex sympathetic dystrophy, addiction, and similar afflictions. In the early cases, it completely ameliorated the depressive condition especially in dementia. These patients felt a sense of well being. The dose administered through the ORE is very small i.e. 500 micrograms compared to systemic administration of 70,000 to 100,000 micrograms, hence no adverse effects. Because of the small dose used to treat the above described neurodegenerative diseases, it has no hallucinogenic effect and did not need the benzodiazepines to counter such effects. Along with the insulin, bexarotene, ketamine, monoclonal antibodies, IGF-1, and cholinesterase inhibitor therapeutic agents compounded to treat Alzheimer's disease to increase the CNS levels of acetylcholine to enhance the memory and cognition in Alzheimer's disease patients. It is important to note that the ketamine easily crosses the BBB. It is formulated as a slightly acid (pH 3.5 to 5.5) sterile solution for intravenous or intramuscular injection in concentrations containing the equivalent of 50 mg ketamine base per milliliter and contains not more than 0.1 mg/mL benzethonium chloride added as a preservative.

Acetylcholinesterase Inhibitors (Acheis) to Increase the Acetylycholine Levels in the Neurons to Improve Memory and Cognition in Alzheimer's Disease

One of the pathgnomonic signs and symptoms characters of the Alzheimer's disease (AD) is that it is linked to a deficiency in the brain neurotransmitter acetylcholine. It is estimated that there is about a 90% loss of acetylcholine in the brains of people suffering from Alzheimer's, which is a major cause of senility with loss of memory. Consequently, acetyl cholinesterase inhibitors (AChEIs) therapy launched for the symptomatic treatment of AD. The prevailing view has been that the efficacy of AChEIs accomplished through their augmentation of acetylcholine-medicated neuron-to-neuron transmission-Communication through synapses. The added benefits of AChEIs are:

a) They enhances the acetylcholine levels in the brain, improving the memory and cognition,

b) They also protect cells in the neuropile from free radical damage,

c) They protect neurons from β-amyloid-induced injury, and

d) They increase the production of antioxidants such as glutathione,

e) Another important effect of these therapeutic agents is that AChEIs directly inhibit the release of cytokines from microglia and monocytes due to increasing the level of acetylcholine. Experimental evidence shows that the acetylcholine suppresses cytokine release through a ‘cholinergic anti-inflammatory pathway’. Hence, prevention of the neuronal damage mediated by cytokines mediated inflammation is one of the important effects of this group of therapeutic agents.

For this reason, the action of AChEIs in AD works not only through the direct acetylcholine-medicated enhancement of neuronal transmission due to increased acetylcholine, but also due to the anti-inflammatory role of these agents as well. AChEIs therapy based on observations that correlate with the cholinergic system abnormalities associated with intellectual impairment seen in Alzheimer's disease. There is also a correlation between areas that have high levels of AChE and degenerative areas in Alzheimer's disease.

The AChEIs inhibitor we selected is physostigmine. There are other therapeutic agents similar to physostigmine can be used instead. We have used Physostigmine to reverse the motor endplate blockade by muscle relaxing agents for 4 decades in anesthesia (Kleinschmidt S, Ziegeler S, Bauer C. Cholinesterase inhibitors. Importance in anesthesia, intensive care medicine, emergency medicine, and pain therapy. Anaesthesist. 2005 August; 54(8):791-9), and it is still in use for the same purpose. It also is used to treat myasthenia gravis, glaucoma, Alzheimer's disease and delayed gastric emptying, orthostatic hypotension and now it is being used to improve short term memory.

It is a tertiary amine (thus does not hydrogen bond, making it more hydrophobic); it can cross the blood-brain barrier. The physostigmine salicylate is used to treat the central nervous system effects of atropine, scopolamine and other anticholinergic drug overdoses, and is the antidote of choice for Datura stramonium and for Atropa belladonna poisoning, the same as for atropine and Gamma-Hydroxybutyric acid (GHB). Physostigmine used to treat GHB effect by producing a nonspecific state of arousal. Physostigmine also has other proposed uses: it could reverse undesired side effects of benzodiazepines such as diazepam, alleviating anxiety and tension. Another proposed use of physostigmine is to reverse the effects of barbiturates.

The mechanism of physostigmine is to prevent the hydrolysis of acetylcholine by the enzyme acetyl cholinesterase (AChE) at the transmitted sites of acetylcholine (Motor endplate and synapses). Physostigmine also has a miotic function, causing pupillary constriction and is useful in treating mydriasis. By this effect, the Physostigmine also increases outflow of the aqueous humor in the eye, making it useful in the treatment of glaucoma.

The systemic use to treat Alzheimer's disease will have an effect on the entire body due to increased acetylcholine all over the body. To prevent systemic effects, we use this therapeutic agent at ORE to have effect locally on the brain, avoid systemic effects, and the dose we use also drastically reduced. Physostigmine Salicylate (physostigmine salicylate injection) Injection is a derivative of the Calabar bean, and its active moiety physostigmine, also known as eserine. It is soluble in water and a 0.5% aqueous solution has a pH of 5.8. Physostigmine Salicylate Injection is available in 2 mL ampules; each mL containing 1 mg of Physostigmine Salicylate in a vehicle composed of sodium metabisulfite 0.1%, benzyl alcohol 2.0% as a preservative in Water for Injection. To lower intraocular pressure (IOP) in an adult with glaucoma, use as sulfate, 0.25% ointment or as salicylate, 0.25% or 0.5% eye drops. The physostigmine is used in small doses with insulin to increase the acetylcholine in the brain to enhance the memory and cognition. It is delivered to the olfactory mucosal neurons without ill effects using ORE.

I. Take physostigmine containing 1 mg/ml. Mix it with 5 ml of normal saline, which gives 200 mcg/ml of the final solution or 10 mcg per drop of the final solution.

II. Deliver 0.50 ml of the final solution of physostigmine in each olfactory mucosal surface (100 mcg). The dose can be increased or decreased depending upon the response.

III. Wait for 5 minutes,

IV. Then administer 0.25 ml of insulin (40 IU per/ml) as described

Monoclonal Antibodies in the Treatment of Alzheimer's Disease with Insulin and Other Therapeutic Agents as Described in this Invention

One widespread unusual feature of neurodegenerative diseases such as Alzheimer's disease is the presence of inflammation, wherein the body recognizes the abnormality of the relevant neuronal tissue and responds to minimize or repair the effects of the abnormality and/or eventually destroy the abnormal tissue (Sandra Amor, Fabiola Puentes, David Baker and Paul van der Valko. Inflammation in neurodegenerative diseases. Immunology, 129 (2010), 154-169; Mark H. DeLegge. Neurodegeneration and Inflammation. Nutrition in Clinical Practice 23 (2008):35-41; Tamy C Frank-Cannon, Laura T Alto, Fiona E McAlpine and Malu G Tansey. Does neuroinflammation fan the flame in neurodegenerative diseases? Molecular Neurodegeneration 2009, 4:47-59; Christopher K. Glass, Kaoru Saijo, Beate Winner, Maria Carolina Marchetto, and Fred H. Gage. Mechanisms Underlying Inflammation in Neurodegeneration. Cell 140 (2010): 918-934; V. Hugh Perry. The influence of systemic inflammation on inflammation in the brain: implications for chronic neurodegenerative disease. Brain, Behavior, and Immunity 18 (2004): 407-413; Marianne Schultzberg, Catharina Lindberg, Asa ForslinAronsson, Erik Hjorth, Stefan D. Spulber, Mircea Oprica. Inflammation in the nervous system-Physiological and pathophysiological aspects. Physiology & Behavior 92 (2007) 121-128; Franke Zipp and Orhan Aktas. The brain as a target of inflammation: common pathways link inflammatory and eurodegenerative diseases. Trends in Neurosciences 29 (9, 2006) 518-527). These are described in detail in U.S. Patent Application Publication Number: 2011/0152967 A1 which are incorporated herein.

The inflammation accompanies not only neurodegenerative disease, but also brain injury such as trauma, stroke, or infection and a host of other slow evolving diseases, also. Consequently, in the methods that are disclosed here; the use of monoclonal antibodies is applicable to any situation in which inflammation in the central nervous system presents a danger to the patient's brain function. Inflammation is modulated by cytokines (Some cytokines may regarded as hormones), which are small cell-signaling protein or peptide molecules that are secreted by glial cells of the nervous system, by numerous cells of the immune system, and by many other cell types. In general, one may adopt two approaches to reduce or prevent inflammation modulated by cytokines. First, one may attempt to inhibit the release or effectiveness of cytokines that promote inflammation. A second approach to reducing inflammation modulated by cytokines is to enhance and/or stimulate the release or effectiveness of cytokines that inhibit inflammation. Antibodies are involved in both these modalities.

Antibodies are proteins, namely immunoglobulins, produced by one B cell lymphocytes in response to specific exogenous foreign antigens. Monoclonal antibodies (mAB), matching immunoglobulin are copies which identify a single specific antigen-cytokine. Monoclonal antibodies against cytokines, act as cytokine inhibitors, antagonists, or as blockers. Tumor necrosis factor (TNF) is a naturally occurring cytokine present in humans in all tissues including the brain, and plays a key role in the inflammatory response and immune reaction in response to infection. Tumor necrosis factor (TNF) formed by the precursor transmembrane protein, forming trimolecular complex soluble molecules, that circulate and bind to receptors found on variety of cells. This binding produces an array of pro-inflammatory effects such as release of other pro-inflammatory cytokines, including IL-6, IL-8, and IL-I; free and discharge matrix metalloproteinases; and up regulation of the expression of endothelial adhesion molecules, further amplifying the inflammatory and immune cascade by drawing white blood cells into extra vascular tissues.

Interleukin-1 is a naturally occurring cytokine, present in mammals and it plays a key role in the inflammatory and the immune responses. Interleukin-1 receptor antagonist (IL-I RA) Kineret (Amgen) is a recombinant form of IL-I RA and is FDA approved for treating rheumatoid arthritis and also be used to treat Alzheimer's disease. The brain of Alzheimer's disease subjected to unspecified inflammatory reactions as described above, resulting in production amyloid beta and neurofibrillary Tau tangles due to this inflammatory component or element in the brain. There are large number of monoclonal antibodies that can counteract these effects. There are multiple TNF antagonists or interleukin-I antagonists enumerated in U.S. Pat. No. 8,119,127 B2, that are included herein. They include, besides others, the following: etanercept (ENBREL®-Amgen); infliximab (RemicadeJohnson and Johnson); D2E7, a human anti-TNF monoclonal antibody (Knoll Pharmaceuticals, Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); pegylated soluble TNF receptor Type I (PEGs TNF-R 1) (Amgen); and onercept, a recombinant TNF binding protein (r-TBP-I) (Serono). Antagonists of interleukin-I include, but are not limited to Kineret® (recombinant ILI-RA, Amgen), ILI-Receptor Type 2 (Amgen) and IL-I Trap (Regeneron).

The latest monoclonal antibodies under study for the treatment of Alzheimer's disease are bapineuzumab and solanezumab. (Found in the article highlighted in Barron's Cover story on Alzheimer's disease. Is Hope Near? By Andrew Bary, Feb. 27, 2012). Bapineuzumab is a humanized monoclonal antibody that acts on the nervous system and has potential therapeutic value for the treatment of Alzheimer's disease (and possibly glaucoma). Regrettably, in patients receiving the highest dose, e.g. 2 mg, MRI scans showed a swelling of the brain tissue due to fluid accumulation (vasogenic edema). No health risks found in subjects receiving either 0.5 or 1 mg of bapineuzumab. When they become available, we plan to use it through the olfactory mucosal delivery system as described in this invention, not through injections or oral routes using no more than 20% of the systemic dose. We will administer only 10-20% of bapineuzumab (100-200 mcg, instead of 1000 to 2000 mcg or higher doses parenteraly), with insulin. This will prevent the formation edema of the brain and at the same time reduce the amyloid β. Solanezumab is another monoclonal antibody being investigated as a neuroprotector for patients with Alzheimer's and will be used at a level of no more than 20% of the systemic dose with insulin. This reduced dose not only decreases the adverse effect, it also reduces the cost. Studies show that the bapineuzumab and solanezumab equally seek to clear the brain of Aβ plaques caused by a protein called beta-amyloid, which accumulates in Alzheimer's patients derived from amyloid precursor protein from the cell membrane. What is not clear is whether clearing the amyloid plaques will have any meaningful benefit in improving the cognition and memory. That is why adding the acetylcholine esterase inhibitors along with these monoclonal antibodies through the ORE as described in this invention will improve memory and cognition. Both these monoclonal antibodies work in unique ways. Bapineuzumab crosses the blood brain barrier and seeks to clear brain cells of amyloid plaque. Solanezumab binds with a precursor of the plaque in the blood, with the aim of prompting the body to pull amyloid plaque from the brain. Our method of olfactory mucosal (ORE, CVVS) delivery will facilitate both these processes with small doses and with the least complications. As the safety dose of this new class of monoclonal antibodies is established, we plan to use both in combinations, so that we can reduce amyloid precursor from the blood and decrease, shrink, and degrade the amyloid plaques, that are already formed in the brain.

The optimism about bapineuzumab stems from Phase II trial results that showed the drug slowed the mental decline in patients who lacked a genetic marker that appears to speed the progression of Alzheimer's. People without the marker make up about 40% of sufferers. The Pfizer group's Phase III clinical trial will study 4,100 people, so that the researchers can evaluate bapineuzumab's effects on a substantial number of patients with and without the genetic marker. Investigators see a 55% chance that the drug will show “modest benefits” in patients without the marker, and see only a 10% chance of any success with the “tougher-to-treat” carriers. Addition of insulin to ORE with bapineuzumab and solanezumab as described in this invention, due to insulin's augmentation and amplifying effects, can change this scenario and make them more effective even with patients with genetic markers and at low doses without complications.

ANAVEX 2-73 is the first of a new class of compounds that act through sigma-1 receptor agonism as well as muscarinic cholinergic effects and modulation of endoplasmic reticulum stress thought to trigger a series of intracellular effects which modify ion channel signaling at the mitochondrial level. It is in the development stage by the Anavex life sciences corporation, which has filed the regulatory submission to begin clinical studies of ANAVEX 2-73. The Phase I study will evaluate the maximum tolerated dose, pharmacokinetics, pharmacodynamics, safety and bioavailability of ANAVEX 2-73. A Phase IIa study in patients with Alzheimer's disease and Mild Cognitive Impairment, currently scheduled to commence, may provide efficacy data as well as further safety data. There are other therapeutic agents such as Gammagard (Baxter), RG7412 (Roche, AC immune), ADO2, (Affiris/Glaxo) ACC/011 (Pfizer, J&J/Elan), and CAD 106 (Novartis) and a host of others at various stages of clinical trials. They are all administered to ORE with insulin to be therapeutically effective in the treatment of Alzheimer's disease and other neurodegenerative diseases with minimum systemic effects as described here.

We have elected to use a potent anti-TNF fusion protein Etanercept for the present; Bapineuzumab and solanezumab be added as the studies progress and available in the market. The other such monoclonal antibodies mentioned above can be also be used instead. Etanercept has many biological effects besides a potent anti-inflammatory agent; it has antiapoptotic effects. In Alzheimer's (Parkinson's and other neurodegenerative diseases including PTSD, stroke, and concussion) diseases, apoptosis plays an important role. Hence, Etanercept, Bapineuzumab, and solanezumab are important in the treatment of Alzheimer's disease and other neurodegenerative diseases as well through ORE delivery described in this inventive method.

Preparation of the Patient and Method of Insertion of Intranasal Delivery Device with Iontophoresis Electrical Impulses Delivery System to the Olfactory Mucosal

Before insertion of the olfactory mucosal (ORE) delivery and Iontophoresis device through the nasal cavity, examination by an ENT specialist for a complete physical check up is in order. The prerequisite for the treatment may include:

a. Patients had previously been diagnosed with Alzheimer's Disease by a neurologist;
b. Patients had no age restriction, and it is required that the patient meet the NINCDS-ADRDA Criteria for probable Alzheimer's disease; also that they meet the DSM-IV criteria for Alzheimer's;
c. All patients be accompanied by a family member or caregiver for therapy;
d. Patients should have a copy of previously performed MRI or CT scan, diagnosing AD,
e. Patients are excluded if they had an active infection, demyelinating diseases such as multiple sclerosis, pregnancy, uncontrolled diabetes mellitus, tuberculosis, history of lymphoma, nasal tumors, or congestive heart failure,
f. Patients with a white blood cell count<3500, hematocrit<27, or a platelet count <120,000 with history of bleeding disorders were excluded.
g. Patients with vascular dementia, and other neurologic disease other than Alzheimer's, were separated,
h. Keep a complete patient records starting from the history, physical examination, then measure vital signs and record any adverse events if any during the procedure. Note the progression of the treatment by patients experience.
i. The patient should not be taking any blood thinning medications,
j. should be free of nasal tumors, and
k. Patients should be without the history of epilepsy, or it should be under control with antiepileptic therapeutic agents.

Testing for Cognition: The primary tests for cognition measured by: using Assessment Scale cognitive subscale (ADAS-Cog); the Severe Impairment Battery (SIB); the Mini-Mental State Examination (MMSE).

Patients evaluated at baseline before treatment, two weekly and monthly subsequently. Patients will let you know whether the treatment is working or not-they are the best evaluator of the effects of therapeutic intervention.

It is also important for the attending physician to examine both sides of the nose with fiber optic nasal scope and inspect the nasal passage, turbinate's, roof of the nose, and ostium of the sphenoid sinus as well ORE. These scopes are flexible, easy to use and to clean. If the patient is sensitive to instrumentation, the use of a local anesthetic spray and KY jelly or similar lubricant will facilitate the examination and insertion of this device. It is important to have an intravenous infusion line open during first insertion-Iontophoresis stimulation, but it may not be needed afterwards when one experiences the safety and simplicity of the therapeutic procedure of this invention. For experimental reasons, the patient connected to EEG, EKG and record before, during, and after the insertion and turning on of the Iontophoresis electrical impulses delivery system of the invention. It may be important to have an anterior-lateral view of x rays of the nose with sphenoid sinus and nasal sinuses. Have emergency first aid equipment at hand.

Placement of the Device and Delivery of Therapeutic Agents and Induction of Iontophoresis to Treat Alzheimer's Disease by the Herein Described Inventive Methods

Once the diagnosis of the Alzheimer's disease is established, and if there are no contraindications for the delivery of therapeutic agents and procedure through the nose, introduce the therapeutic agents as described below through the delivery catheter located in the ORE. Place the patient in supine position with head slightly extended on a neck support. Then start the lontophoresis electrical impulses delivery procedure after carefully positioning the device in on the olfactory mucosa and after administering the therapeutic agents (sphenoid sinus if device has the sphenoid sinus lontophoresis balloon). Use the nasal fiber optic scope to place the device anatomically in the correct position at the desired anatomical sites desirable especially if the tip of the delivery catheter needs to be positioned inside the sphenoid sinus.

Once the device is positioned at the desired anatomical position in the ORE of the nose, instill the therapeutic agents as described in the below examples, and start switching on the electoral output manipulator (FIGS. 5-10, #517) slowly rising the milliamps (mAP) output. Only deliver the milliamps of electrical current the patient tolerates to produce the lontophoresis effect. The threshold amplitude for lontophoresis activation will vary from one patient to the next. To ensure an adequate response, the stimulation parameters adjusted to stimulate at amplitude of about 5-10% below the patient's neuronal activation threshold to about 15-20% over the patient's neuronal activation threshold. The amplitude of the electrical stimulation typically is about 200 micro amps (uA) to about 400-500 milliamps (mA). Other suitable combinations of stimulation amplitude and frequency provided on per patient dependent basis. For example, the electrical stimulation provided by pulse trains of an intermittent duration or continuously, at a frequency of about 10 Hertz (Hz) to about 30 Hertz (Hz), with a pulse width of about 50 microseconds. Set the desired milliamps of electrical current delivered to get the desired therapeutic effects.

INSERTION OF THE DEVICE: Use the lubricating or local anesthetic jelly before introduction of the catheter to slide the catheter with ease. During the insertion, hold the device directed towards the external canthus of the eye abutting against the outer edge of the nose, directing it upwards and backwards. Pass it about 4-5 centimeters and blow the balloon that one can feel by fingers pressing below the edge of the nasal bones just about an inch below the bridge of the nose. Then pass further the device about another 5 centimeters as to place it on the ORE, and the tip close to or into the ostium of sphenoid sinus. Do not pass the device horizontally from the nostril, where the tip will end at the respiratory mucosa depositing the therapeutic agents in the wrong place. The device inserted slowly with the patient lying down with the neck extended with a small support under the patient's neck. The nose sprayed with a local anesthetic and neosynephrine or Afrin™ to shrink the mucus membranes if desired. A cotton ball-wick soaked in local anesthetics and vaso constrictors packed with angled nasal forceps are useful. Antiseptic solutions such as diluted povidone iodine sprayed inside the nasal cavity. As the local anesthetic takes effect, a fiber optic naso scope introduced through the external naris, all the way up to the sphenoethmoidal recesses located at the posterior upper angle of the nose. Then the body of the device guided gently into the sphenoid sinus through the sphenoid foramina. Make sure the patients and caregivers participate during the treatment so that they can carry out the treatment procedure at home.

This invention based on delivering the therapeutic agents to the olfactory mucosa (ORE), and enhancing their uptake with lontophoresis. The device can be used without lontophoresis application, in which case, the device is used to deliver the therapeutic agents to appropriate ORE site (FIGS. 20, 21). The device gives positive results during the stimulation processes of lontophoresis by increasing the memory, recall of the past and remembrance of events as they are happening. This is due to the enhancing of the memory protein generation and activation of the one's that are already inside the neurons by providing electrical impulses needed to transmit the messages from the site of lontophoresis.

The electrical impulses for lontophoresis are delivered continuously or intermittently depending upon the comfort of the patient after instilling the therapeutic agents to the ORE. The electrical impulses switched on and off as needed, according to the improvement in the signs and symptoms and comfort of the patients. The device left in place for hours and more at a time. The device removed to clean, treat with antiseptics, reuse, or replace. The patient put on antibiotics if an infection of the nose and sinuses suspected. First aid supplies should be available in case of emergency including sugar drinks, glucose pills, candy, or colas to counter any accidental development of hypoglycemia due systemic absorption of insulin from respiratory mucosa of the nose (FIG. 1a).

The patients provided with a home kit containing the device described in the FIGS. 14, 20, and 21. They are supplied with complete instructions and appropriate therapeutic agents to be administered. The patient and caregivers instructed to use disposable gloves during insertion. They may need demonstration and practical training in the outpatient room or in the clinic. They need to practice in the clinic plastic mannequin nose to make sure they can use the device effectively without any complications. The drugs should be provided in bottles with clear labeling and instructions when to use them. Instructions given to store the therapeutic agents in refrigerator until use. They are instructed not to freeze the reconstituted therapeutic agents.

### Example 1

### Preparation of Stock Solutions and Method of Olfactory Mucosal Administration

a) Take 150 mg of bexarotene; dissolve it in a solvent such as alcohol, DMSO, Chloroform solvents with suitable carrier such as physiological saline or phosphate buffered saline. We have used DMSO in our study. This solution can contain thickening and solubilizing agents, such as glucose, polyethylene glycol, and polypropylene glycol and mixtures thereof. The final formulation contains 15 mg of bexarotene per ml of solution. The dose delivered to ORE on each side 10, 15, or 30 mg at a time.
b) Then take 100 IU of rapid acting insulin and dilute it in 5 ml of normal saline, in which each ml contains 20 units of insulin. The dose delivered is 5, 15, or 20 IU at a time.
c) Take 2.5 mg of Ketamine, and dilute it in 5 ml of saline, resulting in 0.5 mg per ml or 500 mcg of active ingredient per ml. The dose delivered is 150, 250, or 500 mcg at a time.
d) Take 150 mcg of IGF-1 and dilute in 5 ml of diluents that will provide 30 mcg of Insulin-like growth factor-I (IGF-1) per ml. The dose delivered to the ORE is 15, 30, or 60 mcg at a time.
e) Take physostigmine containing 1 mg/ml. Mix it with 5 ml of normal saline, which gives 200 mcg/ml of the final solution. The dose delivered to the ORE is 100, 200, or 300 mcg at a time.
f) We formulate Etanercept (Embrel) using 400 μg per 5 ml of the diluents solution, which results in 80 μg/ml of the final solution. The dose delivered to the ORE is 40, 80, or 160 μg at a time.

PROCEDURE: Place the patient in supine position with head extended on neck support, and the inventive device inserted and operating,

a) Instill through the syringe 0.25 ml of bexarotene into the delivery catheter to each olfactory mucosal surface drop by drop as shown in FIGS. 5-7. Wait for 30 minutes,
b) then instill 0.25 ml insulin to each olfactory mucosal surface, wait for 15 minutes,
c) Then follow with olfactory mucosal delivery of ketamine, 0.25 ml to each side. Wait for 15 minutes and record the changes,
d) Follow this with 0.25 ml administration of Monoclonal antibodies to each nostril. Wait for another 15 to 30 minutes,
e) Then administer acetylcholine augmenter physostigmine, 0.25 ml from the stock solution. Wait for 15 minutes,
f) Then administer 0.25 ml of IGF-1 to each nostril from the stock solution. Wait for 15 minutes,
g) The iontophoresis device turned ON or OFF any time during the instillation of the therapeutic agents. We turn it on at the beginning of the procedure,
h) The dose of any of these therapeutic agents can be increased or decreased any time during the treatment, depending upon the response to therapeutic agents,
i) Let the patient rest in the supine position for one hour. Take and record the vital signs. Once the patient is stable after observing for at least 90 minutes, send the patient with a caregiver or family attendant or to the patient's room if the person is in the hospital, clinic, or nursing home.

### Example 2

The patients called back one week later to the clinic. They are assessed for memory and cognition changes. The procedure described in example 1 was repeated if there are no complications. They are sent home with the home therapy kit or back to the place of their residence.

### Example 3

The patients called back three weeks later to the clinic. They assessed for memory and cognition improvements, and recorded. The procedure described in example 1 repeated. Then they were sent home with the home therapy kit.

### Example 4

### Home Therapy

Follow the instruction given in Example 1.
a) The patients sent home with a Kit containing insulin, bexarotene, ketamine, Etanercept, IGF-1, and Physostigmine in separate dispensers. They were provided with a special delivery catheter as described in diagrams 20, and 21,
b) The care giver is instructed to place the patient in the supine position with head extended on a neck support, and the inventive device inserted and operating,
c) They were told to use monoclonal antibodies with insulin once a week,
d) and Physostigmine with insulin drops every day and,
e) ketamine with insulin once every three days,
f) The patients instructed to instill bexarotene with insulin once a week,
g) The patients instructed to instill IGF-1 with insulin and every 3 days once.

### Example 5

a) The patients prescribed oral bexarotene 75 mg taken every day for one month instead of intranasal ORE administration. If they develop complications, the dose reduced to 50 mg.
b) Place the patient in supine position with head extended, and the inventive device inserted and operating before delivering the therapeutic agents to ORE every day of the treatment,
c) First day: two hours after taking bexarotene orally, instill to 0.25 ml insulin preparation to each olfactory mucosal surface, wait for 15 minutes to resume activity,
d) Second day: two hours after taking bexarotene orally, Instill 0.25 ml insulin to each olfactory mucosal surface, wait for 15 minutes, then follow with olfactory mucosal delivery of ketamine, 0.25 ml to each side. Wait for 30 minutes in the supine position,
e) Third day: two hours after taking bexarotene orally, Instill 0.25 ml insulin to each olfactory mucosal surface, wait for 15 minutes, and follow this with 0.25 ml administration of Monoclonal antibodies to each nostril. Wait for another 15 to 30 minutes in supine position.
f) Fourth day: two hours after taking bexarotene orally, instill 0.25 ml insulin to each olfactory mucosal surface, wait for 15 minutes, follow this with administer acetylcholine esterase inhibitor (AChEIs) physostigmine, 0.25 ml from the stock solution. Wait for another 15 to 30 minutes in supine position.
g) Fifth day: two hours after taking bexarotene orally, instill 0.25 ml of IGF-1 to each olfactory mucosal surface, and then instill 0.25 ml insulin to each olfactory mucosal surface. Wait for another 15 to 30 minutes in supine position.
h) Sixth day: two hours after taking bexarotene orally, instill 0.5 ml of bexarotene, wait for 30 minutes, followed with insulin to each olfactory mucosal surface. Wait for another 15 to 30 minutes in supine position,
i) Seventh day: two hours after taking bexarotene orally, instill 0.25 ml insulin to each olfactory mucosal surface. Wait for another 15 to 30 minutes in supine position.
j) After end of administering each therapeutic agents each day, let the patient rest in the supine position for another 30-60 minutes. Take the vital signs and send the patient home with a caregiver or family attendant or train the caregiver to administer these therapeutic agents at home setting.
k) The orally administered bexarotene transported to the brain within 2 hours. It reaches maximum therapeutic effective concentration in the CNS by then. Then administer insulin and other therapeutic agents as outlined above every day. Intranasal ORE insulin will augment and amplify the effects of bexarotene in the CNS and effectively reduce the Aβ responsible for the disease.
l) Repeat this cycle every week for a month and evaluate the patient.
m) Stop bexarotene for one week and then restart the therapeutic administration of 75 mg orally again. Discontinue if adverse effects develop; restart only when the symptoms abate with lower oral doses.
n) It is important to keep the thyroid function to the optimum and blood cholesterol levels low.
o) Any combination of the insulin, with bexarotene, ketamine, monoclonal antibodies, IGF-1, and cholinesterase inhibitor therapeutic agents administered. They are administered as a single agent with insulin or as a group of three or more therapeutic agents at a time. The dose adjusted according to the patient's response.

Results: After one month of treatment, the patient's memory and cognition were improved. In some patients, the improvement noticed the same day. Many of them were able to function almost independently. The patients were less depressed and more active with family. Their recall of the past events improved. None of the patients we treated were bedridden. All the patients we treated were in the early stage of Alzheimer's disease (stage. 1 Pre-dementia, Stage. 2 Early-beginning of the AD). Their memory loss improved. They remembered more of the recent event and relied less on memory aids such as reminder notes. Their planning and problem-solving abilities improved. Their completing familiar tasks at home, at work or at leisure were improved. They were less confused with time or place. Their trouble understanding visual images, spatial relationships, and new problems with words in speaking or writing also improved. One important improvement reported was misplacing things and losing the ability to retrace steps. They showed notable improvement on these aspects and used better judgment in solving problems and tasks. They hardly withdrew from work, or social activities, they were involved.

Now, other than physical and mental exercise, only symptomatic therapies for AD are available. Due to multiplicity and difficulty in identifying the etiological factors, it is increasingly clear, that a specific solitary target or pathogenic pathway for the treatment of AD is not yet identified, and it is unlikely to be identified any sooner (Mangialasche F, Solomon A, Winblad B, Mecocci P, Kivipelto M. Alzheimer disease: clinical trials and drug development. Lancet Neurol. 2010 July; 9(7):702-16). Hence, the best strategy in the treatment of Alzheimer's disease is a multi-target therapy as described in this invention. Therefore our inventive multi target therapeutic approach to aim at Aβ, anti neurotoxic agents, inhibition of excitotoxicity pathways, increase neuronal acetylcholine, reduce brain inflammation, and prevent neuronal apoptosis. To these we also include nonsteroidal anti-inflammatory drugs, statins, hormones, vitamin supplements, free-radical scavengers, magnesium L-threonate, Zinc, iron chelation therapy, Metfromin hydrochloride (Glucophage®), progesterone in menopausal woman, Vitamin D₃, B₁₂, B complex, and antiamyloid antibodies (vaccination) when available.

Every patient with senile dementia of all etiologies put on a regimen of half a cup of blue berries twice a day. They were ground in a blender, mixed with vitamin C and orange juice, and drank one hour before any meal. Strawberries were also included during the season. The benefit of blue berries is that they are purchased in bulk during season. Then stored in freezer for many months without losing their antioxidant effects. Many of these patients without any other therapeutic agent's treatment improved their cognition, memory, and fine finger tremors almost ceased or decreased with intake of blueberries. We have used this regimen on Parkinson's also.

We have used hyperbaric Oxygen therapy after ORE administration of therapeutic agents in conjunction in selected cases. This method will not only increase the brain oxygen levels; but also increased atmospheric pressure on the ORE, resulting in enhanced uptake and passage of therapeutic agents into the CNS with ease.

All our patients received Zinc supplement besides magnesium L-threonate as described above. When zinc combined with cysteine, it increases the activity of antioxidant enzymes catalase, glutathione peroxidase, and the antioxidant protein metallothionein. Zinc is the second most abundant trace element in the human body, and it is the most abundant trace element present in the eye. It is essential for the activity of 200 enzymes and for the DNA binding capacity of over 400 nuclear regulatory elements. Zinc functions as an antioxidant by protecting sulfhydryl groups from oxidation, competing with copper and iron to reduce the formation of hydroxyl radicals that are produced as a result of redox cycling and by the induction of the antioxidant protein metallothionein (MT) which can scavenge damaging hydroxyls. Hence, every aging person should receive Zinc as supplement. It is a must in all Alzheimer's patients, taken orally.

The mood and personalities of people with Alzheimer's can change. They can become confused, suspicious, depressed, fearful, or anxious. They may be easily upset at home, at work, with friends or in new places. With the treatment, these symptoms abated or became less of a problem. They were at ease socially and in conversation. Some of the patients showed dramatic improvement within a period of 2 days of therapy.

### ADVANTAGES OF THE PRESENT INVENTION

The advantages of the present invention are multiple. The important ones are, that it provides for the delivery of multiple therapeutic agents to the brain for the treatment of humans (vertebrates, and mammals) with Alzheimer's disease with cognitive impairment bypassing the BBB;

The advantage of the present invention is that it provides for multiple therapeutic agents to be delivered anatomically localized to the olfactory mucosa of the nose resulting in greater efficacy; rapid onset; longer duration of action; improved delivery to the CNS; with fewer or no side effects;

An additional advantage of the present invention is that it allows use of a lower dosage of all therapeutic agents than is routinely administered for the treatment of Alzheimer's disease by administering the above-described therapeutic agents either directly into olfactory mucosa, or in close proximity bypassing the BBB.

A supplementary advantage of the present invention is that due to low doses of therapeutic agents used, it reduces cost and the incidence of undesirable adverse side effects.

A secondary advantage of the present invention is that it provides methods of administration of AChEIs agents, in a human to improve memory and cognitive function in patients with brain pathology directly to the neurons and their synapses.

A main advantage of the present invention is it provides methods of administration of therapeutic agents, which result in improved delivery of multiple therapeutic agents to the CNS for providing suppression and inhibition of the action of TNF in a human with insulin and monoclonal antibodies in combination to improve cognitive function in patients without brain pathology.

A further advantage of the present invention is that it provides methods of administration of NMDA blockers to prevent the excitotoxicity of glutamate and apoptosis of neurons affected.

Another added advantage of the present invention is that it provides for multiple therapeutic agents administered by specific methods delivered to the Alzheimer's disease afflicted site to remove the amyloid beta, reduce its formation, and prevent the neuronal tangles within the neurons, thus reducing or eliminating the etiology.

Another extra advantage of the present invention is that it provides for multiple therapeutic agents administered by specific methods for treating humans with neurological disorders causing cognitive and memory impairment of Alzheimer's disease.

Another supplementary advantage is the proximity of the therapeutic agents close to the pathology results in rapid therapeutic action that will produce speedy clinical improvement in the patient and will give the patient a better opportunity to heal, slow down the disease progression, and prevent further neuropil damage. The method improves the overall mental health of the patient.

Yet another advantage of the present invention is that it provides for multiple therapeutic agents; delivered consecutively, separately, or in combination. They are delivered through the olfactory mucosa, CVVS, CVO, sub Perineural epithelial and nerve fascicular interstitial spaces; delivered to the SAS, CSF and nerve roots to the neuropil as the preferred route, for the treatment of Alzheimer's disease, and other neurodegenerative diseases including PTSD.

Still another advantage of the present invention is; that it provides for therapeutic agents delivered; by retrograde flow by olfactory mucosa, sub Perineural epithelial, and nerve fascicular interstitial spaces, CVVS into the cranial valveless venous system bypassing or circumventing BBB. Thereby facilitating delivery of therapeutic agents directly to the brain for therapeutic purposes where the pathology is in these neurodegenerative diseases.

Another benefit of the present invention is that it provides for therapeutic agents to be delivered by retrograde flow through the olfactory mucosa, sub Perineural epithelial, and nerve fascicular interstitial spaces, CVVS, CVO, into the brain for therapeutic purposes by bypassing the blood-brain barrier in the cranial and spinal cord arterial circulation.

A further advantage of the present invention is that additional therapeutic agents such as vitamin B12, Zinc, vaccinations, immunization therapeutic agents, neurotrophic factors, progesterone etc. besides the ones describe herein can be instilled into the ORE for the treatment of Alzheimer's and other neurological diseases.

An advantage of the present invention is; that it provides for therapeutic agents to be delivered by retrograde flow, through the olfactory mucosa, sub Perineural epithelial, and nerve fascicular interstitial spaces; CVVS, CVO into the brain. The therapeutic agents bypass the blood-brain barrier in the cranial and spinal cord arterial circulation; through the Iontophoresis delivery method to transport large molecular weight therapeutic agents.

Another advantage of this inventive method is that the combination of therapeutic agents described for AD, used to treat other neurodegenerative diseases such as multiple sclerosis, Parkinson's, ALS, senile dementia. This list also includes bipolar disorders, schizophrenia, depression, postpartum depression, autism, anorexia nervosa, obsessive-compulsive disorders (OCD), addiction, spinal cord injury, spinal muscular atrophy, migraine and cluster headaches; neuropathic pain, radiculopathy, low back pain, vertebral disc disease, fibromyalgia, post-herpetic neuralgia, reflex sympathetic dystrophy; and chronic fatigue syndrome, neuropathic pain, Cerebral Palsy, Epilepsy, Essential Tremor, Friedreich Ataxia, Huntington Disease, Hypoxia Brain damage, Lewy Body Disease, PTSD, cerebrovascular disorders such as stroke, Pick's disease, Creutzfeldt Jacob Disease (CJD), Muscular Dystrophies and many other chronic neurodegenerative diseases described above.

Numerous modifications, adjuvants, alternative arrangements of steps explained, and examples given herein may be devised by those skilled in the art without departing from the spirit and scope of the present invention and the appended claims are intended to cover such modifications and arrangements. Thus, the present invention has been described above in detail in connection with what is presently deemed to be the most practical and preferred embodiments of the invention. It will be apparent to those of ordinary skill in the art that numerous modifications, including, but not limited to, variations in size, materials, shape, form, function and manner of procedure, assembly, and use may be made. While the preferred embodiment of the present invention has been described, it should be understood that various changes, adaptations, and modifications may be made thereto. It should be understood, therefore, that the invention is not limited to details of the illustrated invention. Therefore, the present invention shall include embodiments falling within the scope of the appended claims.

1-13. (canceled)

14. An apparatus for electrically stimulating nerves located in an olfactory region, the apparatus comprising
an elongate shaft having a proximal end and an insertion end, the insertion end adapted for placement through an external nasal opening, at an trans-nasal location extending between the exterior nasal opening and an olfactory region, the insertion end comprising an electrode capable of being located adjacent olfactory mucosa with the insertion end located at the trans-nasal location.

15. An apparatus as recited at claim 14 wherein the electrode, when located adjacent the olfactory mucosa, is capable of delivering an electrical impulse that stimulates olfactory nerves located at the olfactory neuroepithelium.

16. An apparatus as recited at claim 14 wherein the electrode, when located adjacent the olfactory mucosa, is capable of delivering an electrical impulse that is conducted through nerves of the olfactory region to nerves of the central nervous system.

17. An apparatus as recited at claim 14 capable of delivering an electrical impulse to stimulate one or more of: the central nervous system; the brainstem; hippocampus; entorhinal cortex; thalamic, hypothalamic, and cerebral cortical centers; and cerebellum.

18. An apparatus as recited at claim 14 comprising an inflatable balloon at a tip of the insertion end, the balloon being locatable within a nasal cavity, adjacent olfactory mucosa, with the insertion end located at the trans-nasal location.

19. An apparatus as recited at claim 14 wherein the electrode is one of multiple bipolar electrodes located along a length of flexible shaft at the insertion end.

20. An apparatus as recited at claim 14 wherein the electrode is located on the flexible shaft, and the flexible shaft does not include an inflatable balloon.

21. An apparatus as recited at claim 14 the insertion end comprising a fluid deliver aperture adapted to be located adjacent olfactory mucosa with the insertion end located at the trans-nasal location.

22. A method of nerve stimulation, the method comprising
providing an apparatus as recited at claim 14,
inserting the insertion end through an exterior nasal opening of a patient and into the nasal cavity to place the electrode adjacent to olfactory mucosa,
delivering an electrical impulse to the electrode.

23. A method as recited at claim 22 wherein the electrical impulse stimulates an olfactory nerve.

24. A method as recited at claim 22 comprising using the electrode to deliver an electrical impulse to stimulate olfactory nerves located at the olfactory neuroepithelium.

25. An method as recited at claim 22 comprising using the electrode to deliver an electrical impulse to nerves of the olfactory region, the impulse then being conducted to nerves of the central nervous system.

26. An method as recited at claim 22 comprising using the electrode to deliver an electrical impulse to nerves of the olfactory region, the impulse then stimulating one or more of: the central nervous system; the brainstem; hippocampus; entorhinal cortex; thalamic, hypothalamic, and cerebral cortical centers; and cerebellum.

27. A method as recited at claim 22 wherein the insertion end comprises a fluid delivery aperture adapted to be located adjacent olfactory mucosa with the insertion end located at the trans-nasal location, the method comprising delivering therapeutic agent to the olfactory mucosa.

28. A method as recited at claim 27 comprising delivering electrical impulses and therapeutic agent to the olfactory mucosa to produce the Iontophoresis effect to enhance the uptake of and transport of large molecule therapeutic agent.

29. A method as recited at claim 27 comprising delivering electrical impulses and therapeutic agent to the olfactory mucosa to produce in a manner to cause electroporation to facilitate uptake of and transport of the therapeutic agent.

30. A method as recited at claim 27 comprising treating a human with Alzheimer's disease.
